(19) United States
(12) Patent Application Publication
(10) Pub. No.: US 2024/0317837 A1
(43) Pub. Date: Sep. 26, 2024

(54) FUSION PROTEINS COMPRISING A PROTEIN WITH PHASE BEHAVIOR

(71) Applicant: ISOLERE BIO, INC., Durham, NC (US)

(72) Inventors: Kelli M. LUGINBUHL, Durham, NC (US); Michael DZURICKY, Durham, NC (US)

(21) Appl. No.: 18/546,061

(22) PCT Filed: Feb. 18, 2022
(86) PCT No.: PCT/US2022/070727

§ 371 Date: Aug. 10, 2023

Related U.S. Application Data
(60) Provisional application No. 63/151,524, filed on Feb. 19, 2021.

(51) Int. Cl. C07K 14/78; C07K 14/005; C07K 14/31; C12N 9/12; C12P 21/02

(52) U.S. Cl. CPC C07K 14/78; C07K 14/005; C07K 14/31; C12N 9/1247; C12P 21/02; C07K 2319/35; C07K 2319/50; C12N 2750/14122; C12N 2770/20022

(57) ABSTRACT

Provided herein are fusion proteins comprising a first polypeptide and a second polypeptide, wherein the second polypeptide has phase behavior. The first polypeptide may be a polypeptide having a desirable biological activity, such as a polypeptide with therapeutic, cosmetic, and/or industrial importance. Fusing the first polypeptide to the polypeptide with phase behavior facilitates purification thereof, and may also help to stabilize the first polypeptide during expression, storage, or after exposure to various conditions known to unfold, degrade, or misfold proteins.

### CROSS-REFERENCE TO RELATED APPLICATIONS

This application claims priority to U.S. Provisional Application No. 63/151,524, filed Feb. 19, 2021, which is incorporated by reference herein in its entirety for all purposes.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: ISOL_006_00US_SeqList_ST25.txt, date recorded: Feb. 19, 2021; file size: 1 megabyte).

### FIELD

The present disclosure is generally related to compositions and methods for purification of biologics. More specifically, the disclosure is related to purification matrices comprising adeno-associated virus-binding polypeptides and methods of using the same.

### BACKGROUND OF THE INVENTION

The use of biologics in medicine and other disciplines is rapidly increasing. Biologics often have high affinity and specificity for a given target, as well as low toxicity and biodegradability. However, their manufacturing and purification can be quite difficult. Biologics, including therapeutic enzymes, antibodies, gene delivery vectors, signaling molecules, hormones, and other proteins, are typically manufactured recombinantly in bacteria, yeast, or mammalian host cells. Numerous downstream purification steps are then required in order to meet acceptable standards of purity (e.g., standards set by the FDA or other regulatory bodies). Host cell proteins, nucleic acids, endotoxins, and viruses are often the main contaminants that must be removed from biologic preparations.

Additionally, for a biologic to remain active, it must maintain its structure. A number of factors may cause a biologic to lose activity, including salt, temperature, lyophilization, oxidative conditions, pH, light, agitation, the orientation of the biologic during storage, freeze-thaw, and a tendency of the biologic to aggregate.

There is a need in the art for improved compositions and methods for manufacturing, purifying, and stabilizing biologics.

### SUMMARY

In some embodiments, provided herein is a fusion protein comprising a first polypeptide and a second polypeptide, wherein the second polypeptide has phase behavior. In some embodiments, the first polypeptide comprises i) an enzyme, or a derivative or catalytic fragment thereof; ii) an antibody, or a derivative or antigen-binding fragment thereof; iii) a signaling molecule, or a fragment or derivative thereof; iv) a structural protein, or a fragment or derivative thereof; or v) a hormone, or a fragment or derivative thereof.

In some embodiments, provided herein is a method for performing a multi-step enzymatic process on a substrate, the method comprising: i) providing a first fusion protein comprising a first enzyme and a polypeptide having a first phase behavior; ii) providing a second fusion protein comprising a second enzyme and polypeptide having a second phase behavior; iii) applying a first environmental factor, which allows the first enzyme to contact, isolate, and/or concentrate the substrate; and iv) applying a second environmental factor, which allows the second enzyme to contact, isolate, and/or concentrate the substrate.

In some embodiments, provided herein is a method for contacting, isolating, and/or purifying a substrate, the method comprising: i) providing a first fusion protein comprising a first enzyme and a polypeptide having a first phase behavior; ii) providing a second fusion protein comprising a second enzyme and polypeptide having a second phase behavior; iii) applying a first environmental factor, which allows the first enzyme to contact, isolate, and/or concentrate the substrate; and iv) applying a second environmental factor, which allows the second enzyme to contact, isolate, and/or concentrate the substrate.

In some embodiments, provided herein is a method for purifying a first polypeptide, the method comprising: i) providing a fusion protein comprising the first polypeptide and a second polypeptide having phase behavior; ii) applying a first environmental factor to the fusion protein; iii) separating the fusion protein aggregates from at least one contaminant on the basis of size and/or density; and iv) applying a second environmental factor to disaggregate the fusion protein.

In some embodiments, provided herein is a method for performing a multi-step enzymatic process on a substrate, the method comprising: i) providing a first fusion protein comprising a first enzyme and a polypeptide having a first phase behavior; ii) providing a second fusion protein comprising a second enzyme and polypeptide having a second phase behavior; iii) applying a first environmental factor, which allows the first enzyme to contact the substrate; and iv) applying a second environmental factor, which allows the second enzyme to contact the substrate.

In some embodiments, provided herein is a method for improving yield of a first polypeptide, the method comprising: i) expressing a fusion protein comprising the first polypeptide and a second polypeptide having phase behavior; and ii) separating the first polypeptide from the second polypeptide, wherein the yield of the first polypeptide is improved when expressed as the fusion protein compared to a yield of the first polypeptide when not expressed as a fusion protein.

In some embodiments, provided herein is a method for substantially preventing loss of activity of a first polypeptide after exposure to one or more conditions known to unfold, degrade, and/or misfold the first polypeptide, the method comprising expressing a fusion protein comprising the first polypeptide and a second polypeptide, wherein the second polypeptide is a polypeptide having phase behavior.

In some embodiments, provided herein is a method for substantially preventing the unfolding, degradation, and/or misfolding of a first polypeptide, the method comprising expressing a fusion protein comprising the first polypeptide and a second polypeptide, wherein the second polypeptide is a polypeptide having phase behavior.

In some embodiments, provided herein is a method for stabilizing a first polypeptide, the method comprising expressing a fusion protein comprising the first polypeptide and a second polypeptide, wherein the second polypeptide is a polypeptide having phase behavior, wherein when the fusion protein is exposed to one or more conditions that would destabilize the first polypeptide, the first polypeptide substantially retains its activity.

In embodiments, the method comprises removing the fusion protein from the conditions and cleaving the first polypeptide from the second polypeptide, wherein the first polypeptide retains its activity compared to a control first polypeptide that has not been exposed to the conditions.

In embodiments, the one or more conditions that unfold, degrade, misfold, or destabilize the first polypeptide comprise: exposure to an oxidizing agent, lyophilization, exposure to non-physiologic pH, exposure to a chaotropic agent, exposure to temperature of at least 50° C., exposure to an organic solvent, exposure to urea, exposure to a detergent, exposure to an autoclave, freeze-thaw cycling, heat shock, or a combination thereof.

In embodiments, the one or more conditions that unfold, degrade, misfold, or destabilize the first polypeptide comprise: exposure to non-physiologic pH. In embodiments, exposure to non-physiologic pH is exposure to acid. In embodiments, the acid is guanidine hydrochloride. In embodiments, exposure to non-physiologic pH is exposure to base. In embodiments, the base is sodium hydroxide or urea.

In embodiments, the one or more conditions known to unfold, degrade, misfold, or destabilize the first polypeptide are exposure to guanidine hydrochloride, exposure to urea, lyophilization, freeze-thaw cycling, autoclaving, exposure to sodium hydroxide, or exposure to temperature of at least 90° C. In embodiments, the one or more conditions known to unfold, degrade, misfold, or destabilize the first polypeptide is exposure to 0.1 M NaOH. In embodiments, the one or more conditions known to unfold, degrade, misfold, or destabilize the first polypeptide is exposure to 0.1 M NaOH for 30 minutes. In embodiments, the one or more conditions known to unfold, degrade, or destabilize the first polypeptide is exposure to 6 M guanidine hydrochloride. In embodiments, the one or more conditions known to unfold, degrade, misfold or destabilize the first polypeptide is exposure to 6 M guanidine hydrochloride for 30 minutes. In embodiments, the one or more conditions known to unfold, degrade, misfold or destabilize the first polypeptide is heating to at least 95° C. In embodiments, the condition is heat shock, and wherein heat shock comprises: heating the fusion protein comprising the first polypeptide to 95° C. for 30 minutes; placing a container containing the fusion protein on ice, and then returning the fusion protein to room temperature.

In embodiments, the fusion protein comprising the first polypeptide is exposed to the one or more conditions for about 15 minutes, about 30 minutes, about 45 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours. In embodiments, exposure to the one or more conditions occurs for at least about 30 minutes. In embodiments, exposure to the one or more conditions occurs for about 30 minutes to about 12 hours, about 30 minutes to about 11 hours, about 30 minutes to about 10 hours, about 30 minutes to about 9 hours, about 30 minutes to about 8 hours, about 30 minutes to about 7 hours, about 30 minutes to about 6 hours, about 30 minutes to about 5 hours, about 30 minutes to about 4 hours, about 30 minutes to about 3 hours, about 30 minutes to about 2 hours, or about 30 minutes to about 1 hours.

In embodiments, the activity of the first polypeptide is its affinity for a binding partner of the first polypeptide. In embodiments, the first polypeptide is an enzyme, and the activity is k_{cat}.

In embodiments, less than 35%, less than 30%, less than 25%, less than 20%, less than 15%, less than 10%, less than 5%, less than 3%, less than 2%, or less than 1% of the activity of the first polypeptide is lost after exposure to one or more of the conditions as compared to a control, wherein the control is not exposed to a condition known to unfold, degrade, or destabilize the first polypeptide. In embodiments, the first polypeptide retains from 65% to 100% of its activity after exposure to one or more of the conditions as compared to a control. In embodiments, the first polypeptide retains at least 65%, at least 70%, at least 75%, at least 80%, at least 85%, at least 90%, at least 95%, or 100% of the its activity after exposure to one or more of the conditions as compared to a control. In embodiments, less than 20% or less than 25% of the activity of the first polypeptide is lost after exposure to one or more of the conditions as compared to a control, wherein the control is not exposed to a condition known to unfold, degrade, or destabilize the first polypeptide. In embodiments, the first polypeptide retains at least 80% of its activity after exposure to one or more of the conditions as compared to a control compared control. In embodiments, the first polypeptide retains its activity at 4° C. for about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 1 month, about 2 months, about 3 months, about 4 months, about 5 months, about 6 months, about 7 months, about 8 months, about 9 months, about 10 months, about 11 months or about 12 months. In embodiments, the first polypeptide retains its activity at −20° C. for about 6 months, about 9 months, about 1 year, about 2 years, about 3 years, about 4 years, about 5 years, about 6 years, about 7 years, about 8 years, about 9 years, or about 10 years.

In embodiments, the yield of the first polypeptide is greater than 15 mg per liter, greater than 30 mg per liter, greater than 50 mg per liter, greater than 75 mg per liter, greater than 100 mg per liter, greater than 200 mg per liter, or greater than 300 mg per liter of host cell suspension. In embodiments, the yield of the first polypeptide in the fusion protein is at least about 50%, at least about 75%, at least about 100%, at least about 125%, at least about 150%, at least about 175%, at least about 200%, at least about 225%, at least about 250%, at least about 275%, at least about 300%, at least about 325%, at least about 350%, at least about 375%, at least about 400%, at least about 425%, at least about 450%, at least about 475%, at least about 500%, at least about 525%, at least about 550%, at least about 575%, at least about 600%, at least about 625%, at least about 650%, at least about 675%, at least about 700%, at least about 725%, at least about 750%, at least about 775%, at least about 800%, at least about 825%, at least about 850%, at least about 875%, at least about 900%, at least about 925%, at least about 950%, at least about 975%, at least about 1000%, at least about 1100%, at least about 1125%, at least about 1150%, at least about 1175%, at least about 1200%, at least about 1225%, at least about 1250%, at least about 1275%, at least about 1300%, at least about 1325%, at least about 1350%, at least about 1375%, at least about 1400%, at least about 1425%, at least about 1450%, at least about 1475%, at least about 1500%, at least about 1525%, at least about 1550%, at least about 1575%, at least about 1600%, at least about 1625%, at least about 1650%, at least about 1675%, at least about 1700%, at least about 1725%, at least about 1750%, at least about 1775%, at least about 1800%, at least about 1825%, at least about 1850%, at least about 1875%, at least about 1900%, at least about 1925%, at least about 1950%, at least about 1975%, or at least about 2000% higher than the yield of a first polypeptide when not expressed as a fusion protein. In embodiments, the yield of the first polypeptide is greater than 75 mg per liter. In embodiments, the yield of the first polypeptide is about 300% higher than the yield of a first polypeptide that is not expressed as a fusion protein.

In embodiments, provided herein is a method for performing an enzymatic process on a nucleic acid substrate, the method comprising: (i) providing a first fusion protein comprising a first enzyme and a first polypeptide having a phase behavior; and (ii) applying a first environmental factor, which allows the first enzyme to contact the substrate. In some embodiments, the method comprises: (iii) providing a second fusion protein comprising a second enzyme and a second polypeptide having phase behavior; (iv) applying a second environmental factor, which allows the second enzyme to contact the substrate. In embodiments, the method comprises: (v) applying a third environmental factor, which separates the first enzyme from the substrate; and (vi) applying a fourth environmental factor, which separates the second enzyme from the substrate. In embodiments, the first enzyme, second enzyme, or both comprises a nucleic acid binding protein (NBP).

These and other embodiments will be further described below in the Detailed Description, Examples, and Claims.

### BRIEF DESCRIPTION OF THE DRAWINGS

FIG. 1 is a graph showing percent fusion protein activity after treatment with various conditions known to unfold, degrade, or misfold proteins including lyophilization, exposure to 0.1M sodium hydroxide (NaOH), 6M guanidine hydrochloride (GuHCl), and even heating to 95° C.

FIG. 2 shows percent fusion protein activity after lyophilization and resuspension in PBS (Untreated), or after lyophilization, autoclaving, and resuspension in PBS (Autoclaved).

FIG. 3 shows percent fusion protein loss after exposure to 0.1M sodium hydroxide, heating to 95° C., and heating to 95° C. at low pH (i.e., pH of 4). After treatment, the fusion protein in each exposure group was neutralized and/or cooled, centrifuged to remove aggregates, and then assayed for total protein using a commercial ELISA. For each group, less than 2% of the starting protein material was lost. Data represent the mean and standard error (n=3).

FIGS. 4A-C shows that a fusion protein comprising the PKD2 domain of the AAV receptor (AAVR) and a polypeptide with phase behavior has superior expression to expression of the PKD2 domain of AA VR in the absence of the polypeptide with phase behavior. FIG. 4A shows the concentrations of PKD2 and fusion protein comprising PKD2 purified per liter. FIG. 4B shows the amount of PKD2 and fusion protein comprising PKD2 purified per liter. FIG. 4C shows expression of PKD2 and the fusion protein comprising PKD2 on a gel.

FIG. 5 shows that a fusion protein comprising the CR3 domain of the LDL Receptor (LDLR) and a polypeptide with phase behavior retains its ability to capture lentivirus after exposure to conditions known to degrade, aggregate, or inactivate polypeptides (e.g., 95° C., 0.1M NaOH incubation, or 6M GuHCl incubation).

### DETAILED DESCRIPTION OF THE INVENTION

### Definitions

As used herein, and in the appended claims, the singular forms “a”, “an”, and “the” include plural referents unless the context clearly dictates otherwise. Thus, for example, reference to “a protein” can refer to one protein or to mixtures of such protein, and reference to “the method” includes reference to equivalent steps and/or methods known to those skilled in the art, and so forth.

As used herein, the term “about” or “approximately” when preceding a numerical value indicates the value plus or minus a range of 10%. For example, “about 100” encompasses 90 and 110.

Also as used herein, “and/or” refers to and encompasses any and all possible combinations of one or more of the associated listed items, as well as the lack of combinations when interpreted in the alternative (“or”).

Unless the context indicates otherwise, it is specifically intended that the various features described herein can be used in any combination.

Moreover, the present disclosure also contemplates that in some embodiments, any feature or combination of features set forth herein can be excluded or omitted. To illustrate further, if, for example, the specification indicates that a particular amino acid can be selected from A, G, I, L and/or V, this language also indicates that the amino acid can be selected from any subset of these amino acid(s) for example A, G, I or L; A, G, I or V; A or G; only L; etc., as if each such subcombination is expressly set forth herein. Moreover, such language also indicates that one or more of the specified amino acids can be disclaimed. For example, in particular embodiments the amino acid is not A, G or I; is not A; is not G or V; etc., as if each such possible disclaimer is expressly set forth herein.

An “adeno-associated virus” (AAV) is a small, replication-deficient parvovirus. As used herein, AAV may refer to a wildtype or mutant AAV of any one of the following serotypes: AAV1, AAV2, AAV3 (including types 3A and 3B), AAV4, AAV5, AAV6, AAV7, AAV8, AAV9, AAV10, AAV11, AAV12, AAV13, AAVrh32.33, AAVrh8, AAVrh10, AAVrh74, AAVhu.68, avian AAV, bovine AAV, canine AAV, equine AAV, ovine AAV, snake AAV, bearded dragon AAV, AAV218, AAV2g9, AAV-LK03, AAV7m8, AAV Anc80, AAV PHP.B, and any other AAV now known or later discovered. In some embodiments, an AAV may have a single-stranded genome, or a double-stranded genome (e.g., a self-complementary AAV).

An “AAV particle” typically comprises a capsid, and a nucleic acid (e.g., a nucleic acid comprising a transgene) encapsidated by the protein capsid. The “capsid” is a near-spherical protein shell that comprises individual “capsid protein subunits” or “capsid proteins” (e.g., about 60 capsid protein subunits) associated and arranged with T=1 icosahedral symmetry. Accordingly, the capsids of the AAV vectors described herein comprise a plurality of capsid proteins. When an AAV particle is described as comprising a capsid protein, it will be understood that the AAV particle comprises a capsid, wherein the capsid comprises one or more AAV capsid proteins. When an AAV particle is described as binding to a binding domain, it will be understood that the binding domain may bind to one or more capsid proteins within the capsid. The term “empty AAV particle” or “empty capsid” refers to an AAV particle or capsid that does not comprise any vector genome or nucleic acid comprising an expression cassette or transgene.

As used herein, the term “AAV sample” used interchangeably herein with “AAV composition” refers to a composition that contains AAV particles. In some embodiments, the “AAV sample” refers to a composition containing AAV of one or more serotypes. For example, an “AAV8 sample” refers to a composition comprising AAV8 particles.

A “viral particle” typically comprises a protein shell (e.g., a capsid or an envelope), and a nucleic acid (e.g., a nucleic acid comprising a transgene) contained therein.

As used herein, the term “fragment” as it refers to a polypeptide includes a truncated form of polypeptide. The fragment has substantially the same activity as the full length protein or polypeptide. For example, a fragment of a nucleic acid binding protein refers to a truncated form of the nucleic acid binding protein that substantially retain its binding affinity for the nucleic acid. As an additional example, a fragment of may include about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 97%, or about 99% of the amino acids of full-length protein. As used herein, the term “substantially” in reference to an activity, such as binding affinity, means that the truncated form as at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% of the activity (e.g., binding affinity) as the full length protein or polypeptide.

As used herein, the term “contaminant” and “impurity” are used interchangeably. A contaminant may refer to any substance that is not desired in a purified composition. In some embodiments, the contaminant is any substance other than the biologic desired to be purified. Non-limiting examples of contaminants include, but are not limited to, a solvent, a protein, a peptide, a carbohydrate, a nucleic acid, a virus, a cell (e.g., a bacterial, yeast, or mammalian cell), a carbohydrate, a lipid, or a lipopolysaccharide. In some embodiments, the contaminant is an endotoxin or a mycotoxin.

As used herein, the terms “peptide,” “polypeptide,” and “protein” are used interchangeably, and refer to a compound comprised of amino acid residues covalently linked by peptide bonds. A protein must contain at least two amino acids, and no limitation is placed on the maximum number of amino acids that can comprise a protein's sequence. The term “peptide” may refer to a short chain of amino acids including, for example, natural peptides, recombinant peptides, synthetic peptides, or a combination thereof. Proteins and peptides may include, for example, biologically active fragments, substantially homologous polypeptides, oligopeptides, homodimers, heterodimers, variants of polypeptides, modified polypeptides, derivatives, analogs, and fusion proteins, among others.

A “polynucleotide” is a sequence of nucleotide bases, and may be RNA, DNA or DNA-RNA hybrid sequences (including both naturally occurring and non-naturally occurring nucleotides). In some embodiments, a polynucleotide is either a single or double stranded DNA sequence.

As used herein, by “isolate” or “purify” (or grammatical equivalents) a viral particle, it is meant that the viral particle is at least partially separated from at least some of the other components in a starting material comprising the viral particle (e.g., a cell lysate). In representative embodiments an “isolated” or “purified” viral particle is enriched by at least about 10-fold, about 100-fold, about 1000-fold, about 10,000-fold or more as compared with the starting material.

As used herein, the term “amino acid” encompasses any naturally occurring amino acid, modified forms thereof, and synthetic amino acids. Naturally occurring, levorotatory (L-) amino acids are shown in Table 1.

TABLE 1
Amino acid residues and abbreviations.

Abbreviation

| | Three-Letter | One-Letter |
|---|---|---|
| Amino Acid Residue | Code | Code |
| Alanine | Ala | A |
| Arginine | Arg | R |
| Asparagine | Asn | N |
| Aspartic acid (Aspartate) | Asp | D |
| Cysteine | Cys | C |
| Glutamine | Gln | Q |
| Glutamic acid (Glutamate) | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Alternatively, the amino acid can be a modified amino acid residue (nonlimiting examples are shown in Table 2) and/or can be an amino acid that is modified by post-translational modification (e.g., acetylation, amidation, formylation, hydroxylation, methylation, phosphorylation or sulfatation).

TABLE 2
Modified Amino Acid Residues.

| | |
|---|---|
| Modified Amino Acid Residue | Abbreviation |

Amino Acid Residue Derivatives

| | |
|---|---|
| 2-Aminoadipic acid | Aad |
| 3-Aminoadipic acid | bAad |
| beta-Alanine, beta-Aminoproprionic acid | bAla |
| 2-Aminobutyric acid | Abu |
| 4-Aminobutyric acid, Piperidinic acid | 4Abu |
| 6-Aminocaproic acid | Acp |
| 2-Aminoheptanoic acid | Ahe |
| 2-Aminoisobutyric acid | Aib |
| 3-Aminoisobutyric acid | bAib |
| 2-Aminopimelic acid | Apm |
| t-butylalanine | t-BuA |
| Citrulline | Cit |
| Cyclohexylalanine | Cha |
| 2,4-Diaminobutyric acid | Dbu |
| Desmosine | Des |
| 2,21-Diaminopimelic acid | Dpm |
| 2,3-Diaminoproprionic acid | Dpr |
| N-Ethylglycine | EtGly |
| N-Ethylasparagine | EtAsn |
| Homoarginine | hArg |
| Homocysteine | hCys |
| Homoserine | hSer |
| Hydroxylysine | Hyl |
| Allo-Hydroxylysine | aHyl |
| 3-Hydroxyproline | 3Hyp |
| 4-Hydroxyproline | 4Hyp |
| Isodesmosine | Ide |
| allo-Isoleucine | aIle |
| Methionine sulfoxide | MSO |
| N-Methylglycine, sarcosine | MeGly |
| N-Methyl isoleucine | MeIle |
| 6-N-Methyllysine | MeLys |
| N-Methylvaline | MeVal |
| 2-Naphthylalanine | 2-Nal |
| Norvaline | Nva |
| Norleucine | Nle |
| Ornithine | Orn |
| 4-Chlorophenylalanine | Phe(4-C1) |
| 2-Fluorophenylalanine | Phe(2-F) |
| 3-Fluorophenylalanine | Phe(3-F) |
| 4-Fluorophenylalanine | Phe(4-F) |
| Phenylglycine | Phg |
| Beta-2-thienylalanine | Thi |

Further, the non-naturally occurring amino acid can be an “unnatural” amino acid.

As used herein, the term “environmental factor” is any factor that, when applied to a composition comprising a protein-based purification matrix, alters one or more properties of the composition. Non-limiting examples of environmental factors include a change in one or more of temperature, pH, salt concentration, concentration of the purification matrix, concentration of the biologic, or pressure; the addition of one or more surfactants, cofactors, vitamins, molecular crowding agents, denaturing agents, reducing agents, or oxidizing agents; or the application of electromagnetic waves.

As used herein, the term “polypeptide with phase behavior” refers to any polypeptide that is capable of undergoing a phase transition. In some embodiments, the polypeptide undergoes a phase transition due to the application of an environmental factor. Exemplary polypeptides with phase behavior include elastin-like polypeptides (ELPs) and resilin-like polypeptides (RLPs).

As used herein, the term “fusion protein” refers to a polypeptide produced when two heterologous nucleotide sequences or fragments thereof coding for two (or more) different polypeptides not found fused together in nature are fused together in the correct translational reading frame.

The term “antibody” refers to an immunoglobulin (Ig) molecule capable of binding to a specific target, such as a carbohydrate, polynucleotide, lipid, or polypeptide, through at least one epitope recognition site located in the variable region of the Ig molecule. As used herein, the term encompasses intact polyclonal or monoclonal antibodies and antigen-binding fragments thereof. For example, a native immunoglobulin molecule is comprised of two heavy chain polypeptides and two light chain polypeptides. Each of the heavy chain polypeptides associate with a light chain polypeptide by virtue of interchain disulfide bonds between the heavy and light chain polypeptides to form two heterodimeric proteins or polypeptides (i.e., a protein comprised of two heterologous polypeptide chains). The two heterodimeric proteins then associate by virtue of additional interchain disulfide bonds between the heavy chain polypeptides to form an Ig molecule. The term “antibody” also includes multispecific antibodies (e.g., bispecific antibodies).

The term “antigen-binding fragment” as used herein refers to a polypeptide fragment that contains at least one complementarity-determining region (CDR) of an immunoglobulin heavy and/or light chain that binds to at least one epitope of the antigen of interest. In this regard, an antigen-binding fragment of the herein described antibodies may comprise 1, 2, 3, 4, 5, or all 6 CDRs of a variable heavy chain (VH) and variable light chain (VL) sequence from antibodies that specifically bind to a target molecule. Antigen-binding fragments include proteins that comprise a portion of a full length antibody, generally the antigen binding or variable region thereof, such as Fab, F(ab′)2, Fab′, Fv fragments, minibodies, diabodies, single domain antibodies (dAb), single-chain variable fragments (scFv), multispecific antibodies formed from antibody fragments, and any other modified configuration of the immunoglobulin molecule that comprises an antigen-binding site or fragment of the required specificity.

As used herein, the term “complementarity determining region” or “CDR” refer to an immunoglobulin (antibody) molecule. There are three CDRs per variable domain: CDR1, CDR2 and CDR3 in the variable domain of the light chain and CDR1, CDR2 and CDR3 in the variable domain of the heavy chain.

The term “F(ab′)2” refers to a protein fragment of IgG generated by proteolytic cleavage by the enzyme pepsin. Each F(ab′)2 fragment comprises two F(ab′) fragments linked by disulfide bonds in the hinge region and is therefore a bivalent antigen-binding fragment. The term “Fab”′ refers to a fragment derived from F(ab′)2 and may contain a small portion of the Fc. Each Fab′ fragment is a monovalent antigen-binding fragment.

The term “F(ab)” refers to two of the protein fragments resulting from proteolytic cleavage of IgG molecules by the enzyme papain. Each F(ab) comprises a covalent heterodimer of the VH chain and VL chain and includes an intact antigen-binding site. Each F(ab) is a monovalent antigen-binding fragment.

An “Fv fragment” refers to a non-covalent VH::VL heterodimer which includes an antigen-binding site that retains much of the antigen recognition and binding capabilities of the native antibody molecule, but lacks the CHI and CL domains contained within a Fab. Inbar et al. (1972) Proc. Nat. Acad. Sci. USA 69:2659-2662; Hochman et al. (1976) Biochem 15:2706-2710; and Ehrlich et al. (1980) Biochem 19:4091-4096.

Binding affinity (Ka) refers to an equilibrium association of a particular interaction expressed in the units of 1/M or M⁻¹. Alternatively, affinity can be defined as an equilibrium dissociation constant (Kd) of a particular binding interaction with units of M. Affinities can be readily determined using conventional techniques (see, e.g., Scatchard et al. (1949) Ann. N.Y. Acad. Sci. 51:660; and U.S. Pat. Nos. 5,283,173, 5,468,614, or the equivalent).

### Fusion Proteins

In some embodiments, the disclosure provides a fusion protein comprising a first polypeptide and a second polypeptide, wherein the second polypeptide has phase behavior. In embodiments, the first polypeptide is an enzyme. In some embodiments, the first polypeptide is any polypeptide having therapeutic, cosmetic, or industrial interest. Also provided herein are methods of stabilizing, purifying, and producing the first polypeptide.

### First Polypeptide

In some embodiments, the disclosure provides a fusion protein comprising a first polypeptide. The first polypeptide may be, for example, any polypeptide having therapeutic, cosmetic, or industrial interest. In some embodiments, the first polypeptide is i) an enzyme, or a catalytic fragment thereof; ii) an antibody, or a antigen-binding fragment thereof; iii) a signaling molecule, or a fragment thereof; iv) a structural protein, or a fragment thereof; v) a hormone, vi) a nucleic acid binding protein (NBP), or a fragment thereof; vii) a therapeutic, or a fragment thereof; viii) a carrier protein, or a fragment thereof; ix) a cytokine, or a fragment thereof; or x) a toxin, or a fragment thereof.

In some embodiments, the first polypeptide is a carrier protein. In embodiments, the carrier protein is selected from: human transcription factor TAF12 (TAF12), ketosteroid isomerase (KSI), maltose binding protein (MBP), beta-galactosidase (β-Gal), glutathione-S-transferase (GST) thioredoxin (Trx), chitin binding domain (CBD), BMP-2 mutant (BMPM), SUMO, CAT, TrpE, Staphylococcal protein A′ streptococcal protein, starch binding protein, cellulose binding domain of endoglucanase A, cellulose binding domain of exoglucanase Cex, biotin binding domain, recA, Flag, poly (His), poly(Arg), poly(Asp), poly(Gln), poly(Phe), poly(Cys), green fluorescent protein, red fluorescent protein, yellow fluorescent protein, cyan fluorescent protein, biotin, anti-Biotin, streptavidin, antibody epitopes, albumin, bovine serum albumin, keyhole limpet hemocyanin, and mutants and fragments thereof. In embodiments, the carrier protein is albumin. In embodiments, the carrier protein is bovine serum albumin.

In embodiments, the first polypeptide is a therapeutic. Non-limiting examples of therapeutics include antibodies, cytokines, lepirudin, cetuximab, dornase alfa, denileukin diftitox, etanercept, bivalirudin, leuprolide, alteplase, interferon alfa-n1, darbepoetin alfa, reteplase, epoetin alfa, salmon calcitonin, interferon alfa-n3, pegfilgrastim, sargramostim, secretin, peginterferon alfa-2b, asparaginase, thyrotropin alfa, antihemophilic factor, anakinra, gramicidin D, intravenous immunoglobulin, anistreplase, insulin (regular), tenecteplase, menotropins, interferon gamma-1 b, interferon alfa-2a (recombinant), coagulation factor Vila, oprelvekin, palifermin, glucagon (recombinant), aldesleukin, botulinum toxin Type B, omalizumab, lutropin alfa, insulin lispro, insulin glargine, collagenase, rasburicase, adalimumab, imiglucerase, abciximab, alpha-1-proteinase inhibitor, pegaspargase, interferon beta-1a, pegademase bovine, human serum albumin, eptifibatide, serum albumin iodinated, infliximab, follitropin beta, vasopressin, interferon beta-1 b, hyaluronidase, rituximab, basiliximab, muromonab, digoxin immune Fab (ovine), ibritumomab, daptomycin, tositumomab, pegvisomant, botulinum toxin type A, pancrelipase, streptokinase, alemtuzumab, alglucerase, capromab, laronidase, urofollitropin, efalizumab, serum albumin, choriogonadotropin alfa, antithymocyte globulin, filgrastim, coagulation factor IX, becaplermin, agalsidase beta, interferon alfa-2b, oxytocin, enfuvirtide, palivizumab, daclizumab, bevacizumab, arcitumomab, eculizumab, panitumumab, ranibizumab, idursulfase, alglucosidase alfa, exenatide, mecasermin, pramlintide, galsulfase, abatacept, cosyntropin, corticotropin, insulin aspart, insulin detemir, insulin glulisine, pegaptanib, nesiritide, thymalfasin, defibrotide, natural alpha interferon/multiferon, glatiramer acetate, preotact, teicoplanin, canakinumab, ipilimumab, sulodexide, tocilizumab, teriparatide, pertuzumab, rilonacept, denosumab, liraglutide, golimumab, belatacept, buserelin, velaglucerase alfa, tesamorelin, brentuximab vedotin, taliglucerase alfa, belimumab, aflibercept, asparaginase Erwinia chrysanthemi, ocriplasmin, glucarpidase, teduglutide, raxibacumab, certolizumab pegol, insulin isophane, epoetin zeta, obinutuzumab, fibrinolysin aka plasmin, follitropin alpha, romiplostim, lucinactant, natalizumab, aliskiren, ragweed pollen extract, secukinumab, somatotropin (recombinant), drotrecogin alfa, alefacept, OspA lipoprotein, urokinase, abarelix, sermorelin, aprotinin, gemtuzumab ozogamicin, satumomab pendetide, albiglutide, antithrombin alfa, antithrombin III (human), asfotase alfa, atezolizumab, autologous cultured chondrocytes, beractant, blinatumomab, C1 esterase inhibitor (human), coagulation factor XIII A-subunit (recombinant), conestat alfa, daratumumab, desirudin, dulaglutide, elosulfase alfa, evolocumab, fibrinogen concentrate (human), filgrastim-sndz, gastric intrinsic factor, hepatitis B immune globulin, human calcitonin, human Clostridium tetani toxoid immune globulin, human rabies virus immune globulin, human Rho(D) immune globulin, human Rho(D) immune globulin, hyaluronidase (human, recombinant), idarucizumab, immune globulin (human), vedolizumab, ustekinumab, turoctocog alfa, tuberculin purified protein derivative, simoctocog alfa, siltuximab, sebelipase alfa, sacrosidase, ramucirumab, prothrombin complex concentrate, poractant alfa, pembrolizumab, peginterferon beta-1a, ofatumumab, obiltoxaximab, nivolumab, necitumumab, metreleptin, methoxy polyethylene glycol-epoetin beta, mepolizumab, ixekizumab, insulin degludec, insulin (porcine), insulin (bovine), thyroglobulin, anthrax immune globulin (human), anti-inhibitor coagulant complex, brodalumab, C1 esterase inhibitor (recombinant), chorionic gonadotropin (human), chorionic gonadotropin (recombinant), coagulation factor X (human), dinutuximab, efmoroctocog alfa, factor IX complex (human), hepatitis A vaccine, human varicella-zoster immune globulin, ibritumomab tiuxetan, lenograstim, pegloticase, protamine sulfate, protein S (human), sipuleucel-T, somatropin (recombinant), susoctocog alfa and thrombomodulin alfa.

In some embodiments, the first polypeptide is a growth factor, such as basic fibroblast growth factor (bFGF), epidermal growth factor (EGF), insulin-like growth factor (IGF1), sonic hedgehog (SHH), bone morphogenic protein 2 (BMP2), glial cell-derived neurotrophic factor (GDNF), or noggin.

In some embodiments, the first polypeptide is a cytokine. In some embodiments, the first polypeptide is an interleukin, such as interleukin-1 (IL-1), interleukin-2 (IL-2), interleukin-3 (IL-3), interleukin-4 (IL-4), interleukin-5 (IL-5), interleukin-6 (IL-6), interleukin-7 (IL-7), interleukin-8 (IL-8), interleukin-9 (IL-9), interleukin-10 (IL-10), interleukin-11 (IL-11), interleukin-12 (IL-12), interleukin-13 (IL-13), interleukin-14 (IL-14), interleukin-15 (IL-15), interleukin-16 (IL-16), interleukin-17 (IL-17), interleukin-18 (IL-18), interleukin-19 (IL-19), interleukin-20 (IL-20) interleukin-21 (IL-21) interleukin-22 (IL-22) interleukin-23 (IL-23), interleukin-24 (IL-24), interleukin-25 (IL-25), interleukin-26 (IL-26), interleukin-27 (IL-27), interleukin-28 (IL-28), interleukin-29 (IL-29), interleukin-30 (IL-30), interleukin-31 (IL-31), interleukin-32 (IL-32), interleukin-33 (IL-33), interleukin-34 (IL-34), interleukin-35 (IL-35), interleukin-36 (IL-36), interleukin-37 (IL-37), interleukin-38 (IL-38), interleukin-39 (IL-39), or interleukin-40 (IL-40). In some embodiments, the cytokine is tumor necrosis factor alpha (TNF-alpha), interferon gamma (IFN-g), granulocyte-macrophage colony-stimulating factor (GM-CSF), or transforming growth factor beta (TGV-B).

In some embodiments, the first polypeptide is a receptor, or a receptor fragment. For example, the first polypeptide may be a cell-surface receptor, or an extracellular portion thereof (e.g., an ectodomain). In some embodiments, the receptor is an ion channel-linked receptor, an enzyme-linked receptor, or a G-protein coupled receptor. In some embodiments, the receptor may be a receptor tyrosine kinase, a tyrosine kinase associated receptor, a receptor-like tyrosine phosphatase, a receptor serine-threonine kinase, a receptor guanylyl cyclase, or a histidine kinase associated receptor.

In some embodiments, the first polypeptide is an enzyme, or a derivative or catalytic fragment thereof. The term “enzyme” as used herein includes proteins, or derivatives or fragments thereof, that are capable of catalyzing chemical changes in other substances without being changed themselves. Non-limiting example of enzymes include oxidoreductases, transferases, hydrolases, lyases, isomerases, ligases, hemicellulases, peroxidases, proteases, gluco-amylases, amylases, alkaline/acid phosphatases, isomerases, oxidases, xylanases, lipases, phospholipases, esterases, cutinases, pectinases, keratanases, reductases, phenoloxidases, lipoxygenases, ligninases, pullulanases, tannases, β-glucosidase, lamarinase, kinases, oxidorectuases, ligases, lysozyme, pentosanases, malanases, glucanases, arabinosidases, hyaluronidase, chondroitinase, dehydrogenase, decarboxylase, kinase, phytase, laccases, and derivatives thereof.

In some embodiments, the enzyme, or a derivative or catalytic fragment thereof, is isolated or derived from bacteria or fungi. In some embodiments, the enzyme, or a derivative or catalytic fragment thereof, is isolated or derived from a mammal.

In some embodiments, the enzyme, or a derivative or catalytic fragment thereof, is a naturally occurring enzyme. In some embodiments, the enzyme, or derivative or catalytic fragment thereof, comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25 or more amino acid mutations, as compared to the naturally occurring enzyme. In some embodiments, the enzyme, or derivative or catalytic fragment thereof, comprises an amino acid sequence with at least 90%, at least 95%, at least 97%, or at least 99% identity to the naturally occurring enzyme.

Unless otherwise indicated, sequence identity is determined using the National Center for Biotechnology Information (NCBI)'s Basic Local Alignment Search Tool (BLAST®), available at blast.ncbi.nlm.nih.gov/Blast.cgi. In some embodiments, the sequence identity is calculated over the entire length of the compared sequences. In some embodiments, the sequence identity is calculated over a 20-amino acid, 50-amino acid, 75-amino acid, 100-amino acid, 250-amino acid, 500-amino acid, 750-amino acid, or 1000-amino acid fragment of each compared sequence.

In some embodiments, the first polypeptide is an antibody, or a derivative or antigen-binding fragment thereof. In some embodiments, the antibody, is rituximab, trastuzumab, retifanlimab, amivantamab, ublituximab, anifrolumab, loncastuximab tesirine, balstilimab, bimekizumab, tralokinumab, evinacumab, sutimlimab, aducanumab, teplizumab, dostarlimab, tanezumab, inolimomab, oportuzumab monatox, narsoplimab, ansuvimab, margetuximab, naxitamab, atoltivimab, maftivimab, and odesivimab-ebgn, belantamab mafodotin, tafasitamab, satralizumab, inebilizumab, sacituzumab govitecan, teprotumumab, isatuximab, eptinezumab, [fam]-trastuzumab deruxtecan, enfortumab vedotin, crizanlizumab, brolucizumab, polatuzumab vedotin, risankizumab, romosozumab, caplacizumab, ravulizumab, emapalumab, cemiplimab, fremanezumab, moxetumomab pasudotox, galcanezumab, lanadelumab, mogamuizumab, erenumab, tildrakizumab, ibalizumab, burosumab, durvalumab, emicizumab, benralizumab, ocrelizumab, guselkumab, inotuzumab, ozogamicin, sarilumab, dupilumab, avelumab, brodalumab, atezolizumab, bezlotoxumab, olaratumab, reslizumab, obiltoxaximab, ixekizumab, daratumumab, elotuzumab, necitumumab, idarucizumab, alirocumab, mepolizumab, evolocumab, dinutuximab, secukinumab, nivolumab, blinatumomab, pembrolizumab, ramucirumab, vedolizumab, siltuximab, obinutuzumab, ado-trastuzumab emtansine, raxibacumab, pertuzumab, brentuximab vedotin, belimumab, ipilimumab, denosumab, tocilizumab, ofatumumab, canakinumab, golimumab, ustekinumab, certolizumab pegol, catumaxomab, eculizumab, ranibizumab, panitumumab, natalizumab, bevacizumab, cetuximab, efalizumab, omalizumab, tositumomab-i131, ibritumomab tiuxetan, adalimumab, alemtuzumab, gemtuzumab, ozogamicin, trastuzumab, infliximab, palivizumab, basiliximab, daclizumab, or a derivative or antigen-binding fragment thereof, with at least 90%, at least 95%, at least 97%, or at least 99% identity to any one of the aforementioned antibodies or antigen-binding fragments thereof. In some embodiments, the antibody or a derivative or antigen-binding fragment thereof, comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25 or more amino acid mutations, as compared to any one of the aforementioned antibodies or antigen-binding fragments thereof.

In some embodiments the first polypeptide is a signaling molecule, or a fragment or derivative thereof. The term “signaling molecule” refers to a protein that causes a cell to undergo a process that entails a defined sequence of biochemical reactions within the cell within a cell. Non-limiting examples of signaling molecules include receptor tyrosine kinases (e.g., G protein coupled receptors), nuclear hormone receptors, extracellular signal-regulated kinase (ERK), vaccinia virus (VHR) H1-related protein, a member of the mitogen-activated protein kinase (MKP) family of phosphatases, an interleukin, a cytokine, a transcriptional activator, or a transcription factor. In some embodiments, the signaling molecule, or a fragment or derivative thereof, is a naturally occurring signaling molecule. In some embodiments, the signaling molecule, or a fragment or derivative thereof, comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25 or more amino acid mutations, as compared to the naturally occurring signaling molecule, or a fragment or derivative thereof. In some embodiments, the signaling molecule, or a fragment or derivative thereof, comprises an amino acid sequence with at least 90%, at least 95%, at least 97%, or at least 99% identity to the naturally occurring signaling molecule, or a fragment or derivative thereof.

In some embodiments the first polypeptide is a structural protein, or a fragment or derivative thereof. The term “structural protein” refers to a class of non-catalytic proteins that may serve as a biological structural support. The proteins may serve as biological structural supports by themselves, in conjunction with other proteins, or as a matrix or support for other materials. Non-limiting examples of structural proteins include spider silks, porins (e.g., outer membrane porin F precursor), keratin, collagen, actin, actinin, aggrecan, biglycan, cadherin, clathrin, decorin, elastin, fibrinogen, fibrin, heparin, laminin, mucin, myelin associated glycoprotein, myelin basic protein, myosin, spectrin, tropomyosin, troponin, tubulin, vimentin, vitronectin, and recognin. In some embodiments, the aforementioned structural protein, or a fragment or derivative thereof, comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25 or more amino acid mutations, as compared to the naturally occurring structural protein, or fragment or derivative thereof. In some embodiments, the structural protein, or a fragment or derivative thereof, comprises an amino acid sequence with at least 90%, at least 95%, at least 97%, or at least 99% identity to the naturally occurring structural protein, or a fragment or derivative thereof.

In some embodiments the first polypeptide is a hormone, or a fragment or derivative thereof. The term “hormone” refers to a chemical released by a cell, gland, or organ in one part of an organism that sends out messages that affect cells in other parts of the organism. Non-limiting examples of hormones include thyrotropin-releasing hormone, corticotrophin-releasing hormone, growth-hormone releasing hormone (GHRH), dopamine, somatostatin, vasopressin, growth hormone, thyroid stimulating hormone, adrenocorticotrophic hormone (ACTH), follicle-stimulating hormone (FSH), melanocyte-stimulating hormone (MSH), luteinizing hormone (LH), prolactin, oxytocin, thymopoietin, IGF, THPO, androgens, glucocorticoids, aldosterone, adrenaline, noradrenaline, estrogen, progesterone, prolactin, relaxin, melatonin, calcitonin, PTH, gastrin, ghrelin, histamine, neuropeptide Y, insulin, glucagon, calcitriol, renin, erythropoietin, inhibin, and calcitonin. In some embodiments, the hormone is a growth factor.

In some embodiments, the first polypeptide is a mammalian polypeptide. The mammalian polypeptide may be a human polypeptide, a goat polypeptide, a rabbit polypeptide, a mouse polypeptide, a rat polypeptide, a primate polypeptide, or a baboon polypeptide.

In some embodiments, the first polypeptide is a viral polypeptide. Non-limiting examples of viral polypeptides include the gag protein, DNA polymerase, a protease, a capsid protein, an envelope polypeptide, a fusion polypeptide, and a spike protein. In some embodiments, the viral polypeptide is a capsid protein. In some embodiments, the viral polypeptide is an envelope protein. In some embodiments, the viral polypeptide is a spike protein. In some embodiments, the capsid protein is an AAV capsid protein. In some embodiments, the viral polypeptide is a bacteriophage polypeptide, such as T7 polymerase.

In some embodiments, the first polypeptide is a bacterial polypeptide, or derivative or fragment thereof. The term “bacterial polypeptide” refers to a polypeptide or protein naturally produced by bacteria or other microorganisms. In In some embodiment, the bacterial polypeptide is botulinum toxin, diphtheria toxin, anthrax toxin, pseudomonas exotoxin A, or Shiga toxin. In some embodiments, the bacterial polypeptide is Staphylococcus protein A (SPA) or protein L.

In some embodiments, the first polypeptide is a toxin. Non-limiting examples of toxins include the Heat labile toxin (LT), Heat stabile toxin (ST), Verotoxins, shiga-like toxins (Stxs), Cytotoxins, endotoxins (e.g., lipopolysaccharide (LPS)), EnteroAggregative ST toxin (EAST), Shigella enterotoxins 1 (ShET1), Shigella enterotoxins 2 (ShET2), Neurotoxin, Cytolethal distending toxins (Cdt), AvrA toxin, Cytotoxic necrotizing factors, murine toxin, cytolethal distending toxins, AvrA toxin, toxin complex, cytotoxin necrotizing factor, Yst toxin, heat stabile toxin, Shiga-like toxin II, leukotoxin, enterotoxin, a heat-stable like enterotoxin, extracellular toxic complex, hemolysin, pore-forming toxin, α-hemolysin, heat-stable like toxin, extracellular toxin complex (ETC), alpha-toxin, beta toxin, a neurotoxin, C2 toxin, tetanus neurotoxin, epsilon-toxin, tetanospasmin, lecithinase, cholera toxin, accessory cholera enterotoxin, repeats-in-toxin (RTX) toxin, C3 toxin, iota-toxin, theta-toxin, δ-hemolysin, δ-hemolysin, γ-hemolysin, panton-valentine leukocidin, staphylococcal enterotoxins, toxic shock syndrome toxin-1, zona occludens toxin, cholix toxin, Cholera toxins (CTx), accessory cholera enterotoxin (Ace), RTX toxin, zona occludens toxin (Zot), Cholix toxin, β-haemolysin/cytolysin, CAMP factor, Streptolysin O, Streptolysin S, Pneumolysin, an exotoxin, vacuolating cytotoxin A (VacA), Cytolytic toxins, exotoxin A, exotoxin S, exotoxin T, exotoxin U, exotoxin Y, phospholipase C, a Cry toxin, an anthrax toxin, cytolethal distending toxin A, cytolethal distending toxin B, cytolethal distending toxin C, cholera-like enterotoxin, adenylate cyclase toxin, pertussis toxin, tracheal cytosoxin, dermonecrotic toxin, diptheria toxin, Bacteroides fragilis toxin, and listerolysin O. In some embodiments, the toxin is botulinum toxin, diphtheria toxin, anthrax toxin, pseudomonas exotoxin A or Shiga toxin.

In some embodiments, the toxin is isolated from a bacteria, for example, a bacteria selected from any one of the genus: Yersinia Salmonella, Shigella, Escherichia, Enterobacter, Klebsiella, Serratia, Proteus, Citrobacter, Clostridium, Vibrio, Staphylococcus, Streptococcus, Helicobacter, Pseudomonas, Pasteurella, Bacillus, Campylobacter, Aeromonas, Neiserria, Bordetella, Haemophilus, Chlamydia, Corynebacteria, Bacteroides, Corynebacteria, and Listeria.

In some embodiments, the first polypeptide is an antigenic polypeptide. The term “antigenic polypeptide” refers to any polypeptide that elicits an immune response in an organism. In some embodiments, an antigenic polypeptide results in the development of a humoral and/or a cellular immune response to the antigenic polypeptide. In some embodiments, the antigenic polypeptide is a component of a vaccine. In some embodiments, the antigenic polypeptide is selected from hemagglutinin, spike protein, neuraminidase, hepatitis B surface antigen (HBsAg), a fusion protein, or a capsid protein. In some embodiments, the antigenic polypeptide is the spike protein from SARS-COV or the spike protein from SARS-COV-2.

In some embodiments, the first polypeptide is an enzyme capable of performing one or more steps involved in protein synthesis. Non-limiting examples of enzymes involved in protein synthesis include ribosomal proteins, such as proteins encoded by any one of the following genes: RPSA, RPS3, RPS3A, RPS4X, RPS4Y, RPS5, RPS6, RPS7, RPS8, RPS9, RPS10, RPS11, RPS12, RPS13, RPS14, RPS15, RPS 15A, RPS16, RPS17, RPS18, RPS19, RPS20, RPS21, RPS23, RPS24, RPS25, RPS26, RPS27, RPS27A, RPS28, RPS29, RPS30, RPL3 RPS14, RPL5, RPL6, RPL7, RPL7A, RPL8, RPL9, RPL10, RPL10A, RPL12, RPL13A, RPL14, RPL15, RPL17, RPL18, RPL18A, RPL19, RPL21, RPL22, RPL23, RPL23A, RPL24, RPL26, RPL27A, RPL30, RPL31, RPL32, RPL34, RPL25, RPL26, RPL36A, RPL37, RPL39, RPL50, RPP0, RPP1, RPP1.

In some embodiments, the first polypeptide participates in protein folding, such as a chaperone protein. Non-limiting examples of chaperone proteins include heat shock proteins such as Hsp70 (cpn60, GroEL), Hsp60 (DNAK, BiP), Hsp25, HSP90 (Clp), Calnexin, calreticulin, PDI, PPI, alpha-lytic protease, or subtilisin.

In some embodiments, the first polypeptide is an enzyme capable of performing one or more steps involved in protein modification. For example, the first polypeptide may be a kinase, a phosphatase, a methylase, a glycosyltransferase, an enzyme that adds or removes lipids, a capping enzyme, or a tailing enzyme. In some embodiments, the first polypeptide is involved in post-translational modification, such an enzyme involved in phosphorylation, glycosylation, S-nitrosylation, methylation, n-acetylation, palmitoylation, n-myristoylation, prenylation, sumoylation, or ubiquitination. In some embodiments, the first polypeptide is involved in phosphorylation, methylation, lipidation, capping, or tailing. In some embodiments, the first polypeptide is a kinase or a phosphorylase. In some embodiments, the enzyme is AMAN1, MGAT/GNT1, AMAN II, MGAT2/GNT II, MGAT3/GNT III, MGAT4A/GNT IV, MGAT5/GNT V, FUT8, B4GALT1, ST3GAL3, ST3GAL1, FUT11, XYLT, POMT1, POMT2, GALNT1, POFUT1, XYLT1, HPAT1, HPAT3, GALT2, SERGT1, or RRA1.

In some embodiments, the first polypeptide is an enzyme capable of performing one or more steps involved in DNA or RNA synthesis. For example, the first polypeptide may be a polymerase, such as a DNA or an RNA polymerase. In some embodiments, the first polypeptide may be a helicase.

In some embodiments, the first polypeptide is an enzyme capable of performing one or more steps involved in DNA or RNA modification. For example, the first polypeptide may be a Cas enzyme, such as Cas9 or Cas12. In some embodiments, the first polypeptide may be a Zn finger nuclease. In some embodiments, the first polypeptide may be a TALEN. In some embodiments, the first polypeptide may be a meganuclease. In some embodiments, the first polypeptide may be a deaminase.

In some embodiments, the first polypeptide is a nucleic acid binding protein (NBP). In some embodiments, the nucleic acid binding protein is an RNA binding protein (RBP). In some embodiments, the nucleic acid binding protein is a DNA binding protein (DBP). RBPs bind to RNA whereas DBPs bind to DNA. In embodiments, the NBP binds to DNA, a microRNA, capped RNA, DNA, double stranded RNA, transfer RNA, ribosomal RNA, a small nuclear RNA, a regulatory RNA, a ribozyme, a transfer RNA, or a messenger RNA.

In embodiments, the NBP binds to a poly A tail, a double stranded RNA, an AU-rich element (ARE), a positively charged intrinsically disordered region (IDR) of a nucleic acid, or an mRNA cap.

In embodiments, an RBP binds to an AU-rich element. AU-rich elements are referred to herein as “ARE”. An ARE refers to an adenylate-uridylate-rich element in the 5′ or 3′ untranslated region of a mRNA. AREs contain the core sequence AUUUA (SEQ ID NO: X). AREs are a determinant of RNA stability, and often occur in mRNAs of proto-oncogenes, nuclear transcription factors, and cytokines. Proteins that bind to ARE are referred to as ARE-binding proteins (ARE-BP). In embodiments, ARE-BP stabilize mRNA. Non-limiting examples of ARE-BP include human antigen R (huR, also called “ELAV”), tristetrapolin (TTP), AU-rich element RNA-binding protein (AUF), and fragile X mental retardation syndrome-related protein 1 (FXR1). The following articles describe ARE-BP and are incorporated by reference herein in their entirety: Otsuka et al. Front. Genet., 2 May 2019; Brennan and Steitz. Cell Mol Life Sci. 2001 February; 58(2):266-77; Carballo et al. 1998. Science, 281, 1001-1005; Mazan-Mamczarz et al. Oncogene volume 27, pages 6151-6163 (2008); Vasudevan and Steitz. Cell. 2007 Mar. 23; 128(6):1105-18; Curr Cancer Drug Targets. 2019; 19(5):382-399; J Biol Chem. 2017 Apr. 28; 292(17):6869-6881. doi: 10.1074/jbc.M116.772947. Epub 2017 Mar. 16; Wiley Interdiscip Rev RNA. July-August 2014; 5(4):549-64. doi: 10.1002/wrna.1230; and Elife. 2017 Aug. 2; 6:e26129. doi: 10.7554/eLife.26129; Mazan-Mamczarz et al. 2008. Nucleic Acids Research, 37, 204-214. In embodiments, an NBP binds to an ARE. In embodiments, the NBP binding to an ARE incorporates a binding element of huR, TTP, AUF, or FXR1.

In embodiments, an NBP binds to double stranded RNA. In embodiments, the NBP comprises a dsRNA binding protein (dsRBD) or a fragment thereof. dsRBDs are described in the following reference which is incorporated by reference herein in its entirety: Banerjee et al. RNA Biol. 2014 October; 11(10): 1226-1232.

In embodiments, an NBP binds to capped mRNA. In embodiments, the NBP binding to capped mRNA comprises eukaryotic translation initiation factor 4E (eIF4E), eukaryotic translation initiation factor3 Subunit D (eIF3D), or a combination thereof.

In embodiments, the NBP binds to a groove of DNA or RNA. Non-limiting examples of nucleic acid binding proteins that bind to the groove of DNA or RNA include the trans-activator of transcription (Tat) protein of human immunodeficiency virus-1 (HIV-1), the REV protein of HIV-1, and the RSG-1.2 peptide. RSG-1.2 peptide is a synthetic peptide which binds to the Rev responsive element present within the env gene of the HIV-1 genome. The RSG-1.2 peptide is described in the following article, which is incorporated by reference herein in its entirety: Kumar et al. PLOS One. 2011; 6(8):e23300.

In embodiments, the NBP binds to mRNA. In embodiments, the NBP that binds to mRNA is a ribosomal protein. In embodiments, the ribosomal protein is a 70S ribosome or a 80S ribosome. In embodiments, the ribosomal protein is from the 40S or 60S subunit of the 80S ribosome. In embodiments, the ribosomal protein is from the 30S or 50S subunit of the 70S ribosome. In embodiments, the ribosomal protein is selected from the group consisting of the L3 ribosomal protein, the L4 ribosomal protein, the L13 ribosomal protein, the L20 ribosomal protein, the L22 ribosomal protein, the L24 ribosomal protein, the L24e ribosomal protein, the S12 ribosomal protein, the S14 ribosomal protein, and the eukaryotic initiation factor 4E-binding protein 1 (4EBP1).

In embodiments, the NBP that binds to mRNA is part of the spliceosome. In embodiments, the NBP that is part of the spliceosome is a splicing factor. In embodiments, the splicing factor is selected from the ASF/SF2 splicing factor, serine/arginine rich splicing factor 4 (SRp75), and the serine and arginine rich splicing factor 1 (SRSF1).

In embodiments, the NBP that binds to mRNA is a protein that localizes to p-granules. In embodiments, the protein that localizes to a p-granule is selected from the group consisting of LAF-1, MEG-1, and MEG-3. LAF-1, MEG-1, and MEG-3 are described in the following references, which are incorporated by reference herein in their entirety: Leacock et al. Genetics, Volume 178, Issue 1, 1 Jan. 2008, Pages 295-306; Wu et al. Mol Biol Cell. 2019 Feb. 1; 30(3): 333-345; Elbaum-Garfinkle et al. Proc Natl Acad Sci USA. 2015 Jun. 9; 112(23):7189-94.

In embodiments, the NBP that binds to mRNA is a protein that removes or facilitates removal of the 5′ cap of mRNA, referred to herein as a “decapping protein.” In embodiments, the protein that removes or facilitates removal of the 5′ cap of mRNA is Dcp1, Dcp2, or a combination thereof. Dcp1 and Dcp2 are described in the following reference which is incorporated by reference herein in its entirety: Valkov et al. Nature Structural & Molecular Biology volume 23, pages 574-579 (2016).

In embodiments, the NBP that binds to mRNA is a component of a processing body (p-body). In embodiments, the component of a p-body is Edc3, DHX9, or Xrn1. Components of p-bodies are described in the following reference which is incorporated by reference herein in its entirety: Luo et al. Biochemistry 2018, 57, 17, 2424-2431.

In embodiments, the NBP that binds to mRNA is stem-loop binding protein (SLBP). SLBP binds to the histone 3′ untranslated region (UTR) stem loop structure in replication-dependent histone mRNAs. In embodiments, the NBP that binds to mRNA is a heterogenous nuclear ribonucleoprotein (hnRNP). hnRNPs are described in the following reference which is incorporated by reference herein in its entirety: Geuens et al. Hum Genet. 2016; 135: 851-867.

In embodiments, the NBP that binds to mRNA is GroEL.

In embodiments, the NBP is a protein involved in in vitro transcription. Non-limiting examples of NBPs involved in in vitro transcription include T7 RNA polymerase, Rnase inhibitor, 2′-O-Methyltransferase, Inorganic Pyrophosphatase, Poly(A) Polymerase, DNase I, Calf intestinal phosphatase, Antarctic phosphatase, D1 subunit of the Vaccinia virus mRNA capping enzyme, Guanine-7-methyltransferase (found in D1 subunit of the Vaccinia virus mRNA capping enzyme), Guanylyltransferase (found in D1 subunit of the Vaccinia virus mRNA capping enzyme), RNA triphosphatase (found in D1 subunit of the Vaccinia virus mRNA capping enzyme), and D12 subunit of vaccinia virus mRNA capping enzyme. The following references describe the aforementioned proteins and are incorporated by reference herein in their entirety: Dickson et al. Prog Nucleic Acid Res Mol Biol. 2005; 80: 349-374; Shuman et al. J Biol Chem. 1980 Dec. 10; 255 (23):11588-11598; Luo et al. J Virol. 1995 June; 69(6): 3852-3856; Kobori et al. PNAS Nov. 1, 1984 81 (21) 6691-6695.

In embodiments, the NBP is selected from the group consisting of poly(A)-binding protein (PABP), eukaryotic translation initiation factor 4E (eIF4E), eukaryotic translation initiation factor3 Subunit D (eIF3D), heterogenous nuclear ribonucleoproteins (hnRNPs), RNA-specific adenosine deaminase 1 (ADAR1), RNA-specific adenosine deaminase 2 (ADAR2), CspB from Bacillus subtilis (Bscscp), Y-box protein 1 cold shock domain (YB1-CSD), a Fox-1 protein (FOX1), poly(A)-binding protein (PABP), Staufen protein, TIS11d, zinc finger protein (ZNF), Z-DNA binding protein 1 (ZBP1), retinoic acid-inducible gene-I (RIG-I) like protein, toll like receptor 7 (TLR7), toll like receptor 8 (TLR3), toll like receptor 8 (TLR8), retinoic acid-inducible gene I (RIG-I), melanoma differentiation-associated protein 5 (MDA5), interferon induced protein with tetratricopeptide repeats 1 (IFIT1), protein kinase R (PKR), 2′-5′-oligoadenylate synthetase, an oligoadenylate synthase-like (OASL) protein (e.g., OAS1, OAS2, OAS3, or OASL), ribonuclease E (RNASE E), gamma-interferon-inducible protein Ifi-16 (IF116), and cyclic GMP-AMP synthase (cGAS). The following references describe select aforementioned proteins and are incorporated by reference herein in their entirety: Kuroyanagi. Cell Mol Life Sci. 2009; 66(24): 3895-3907; Baou et al. J Biomed Biotechnol. 2009; 2009: 634520; and Brisse and Ly. Front. Immunol., 17 Jul. 2019; 10: 1586; Rehwinkel et al. Nature Reviews Immunology volume 20, pages 537-551 (2020); and Brisse et al. Front Immunol. 2019; 10: 1586; Luo et al. Cell. 2011 Oct. 14; 147(2): 409-422.

In embodiments, the NBP comprises one or more RNA binding domains (RBDs) and one or more intrinsically disordered regions (IDRs). In embodiments, the IDR comprises an RG[G] repeat, an RS/RG rich domain, a K/R patch, molecular recognition features, a low complexity sequence, a pentatricopeptide domain, or a combination thereof.

In embodiments, the NBP comprises one or more of the following domains: a short linear motif (SLIM), an RG repeat, an RGG repeat, a RS/RG rich domain, a K/R basic patch, a molecular recognition feature, a low complexity sequence, an RNA recognition motif, a double-stranded RNA binding domain, a K homology domain, a zinc finger domain (e.g., CCHH ZF domain, a CCCC (Ran-BP2) domain, a CCCH ZF domain), an RGG domain, a Pumillo family domain, a pentatricopeptide domain, a cold shock domain, a helicase domain, a La motif, a Piwi-Argonaute-Zwille (PAZ) domain, a P-element induced wimpy testis, a pseudouridine synthase and archaeosine transglycosylate (PUA), a Pumillo-like repeat (PUM), a ribosomal S1-like (S1), Sm and Like-Sm (Sm/Lsm) repeat, thiouridine synthases and RNA methylases and pseudouridine synthases (THUMP), and a domain with YT521-B homology. The following references describe many of these domains and are incorporated by reference herein in their entireties: Balcerak et al. Open Biol. 2019 June; 9(6) 190096; Jarvelin et al. Cell Commun Signal, 2016: 14, 9; Corley et al. Mol. Cell. 2020 Apr. 2; 78(1): 9-29; De Franco et al. Sci Rep: 2019: 9, 2484; Shotwell et al. 2020. Wiley Interdiscip Rev RNA, 11, e1573; Simon et al. 2019. Molecular Cell, 75, 66-75.e5; Varadi et al, 2015, PLOS One, 10, e0139731; Zeke et al, 2020, WIREs RNA, n/a, e1714.

In embodiments, the NBP comprises a short linear motif (SLIM). A SLIM is composed of up to ten amino acid residue motifs located predominantly outside protein domains. SLIMs bind to RNA with low affinity in a non-specific manner. SLIMs are often repeated multiple times throughout a protein.

In embodiments, the NBP comprises an RS/RG rich domain. RS/RG rich domains contain repeats of arginine-serine (RS), arginine-glycine (RG), or a combination thereof. RS/RG rich domains mediate specific or non-specific interactions with RNA. Examples of proteins containing RS/RG rich domains include the SR proteins and SR-like proteins like serine/arginine-rich splicing factor 1 (SRSF1) and RNA-helicase DDX23.

In embodiments, the NBP comprises a RG[G] repeat. RG[G] repeats are known to have broad, degenerate binding. RG[G] repeats are motifs rich in arginine and glycine consisting of at least three RG/RGG repeats (e.g., from 3-500), separated by 10 amino acid residues. RG/RGG motifs include RGG and/or RG repeats of varied lengths interspersed with spacers of different amino acids. In embodiments, an NBP comprises a di-RGG motif. Di-RGG motifs contain two repeated RGG sequences separated by 0-4 amino acids. In embodiments, an NBP comprises a di-RG motif. Di-RG motifs contain two repeated RG sequences separated by 0-4 amino acids. In embodiments, an NBP comprises a tri-RGG motif. Tri-RGG motifs contain three repeated RGG sequences separated by 0-4 amino acids. In embodiments, an NBP comprises a tri-RG motif. Tri-RG motifs contain three repeated RG sequences separated by 0-4 amino acids. These motifs are described in the following article which is incorporated by reference herein in its entirety: Thandapani et al. (2013). Molecular Cell, 50, 613-623.

In embodiments, the amino acid sequence of the NBP comprises one or more RG, RGG, RGGR, RGGGR, or a combination thereof. In embodiments, NBPs comprising RG, RGG, RGGR, or RGGGR or a combination thereof mediate hydrogen bonding and base stacking with DNA and RNA via the arginine moieties. In embodiments, NBPs comprising RG, RGG, RGGR, RGGGR or a combination thereof bind to DNA G-quadruplexes. An exemplary protein containing a repeat of RGG, RGGR, or RGGGR is the RNA binding protein FUS. In embodiments, the NBP comprises FUS. In embodiments, the NBP sequence contains consecutive repeats of RGG, RGGR, RGGGR, or combinations thereof. An exemplary NBP containing a combination of RGG, RGGR, or RGGGR repeats may comprise the sequence RGGRGGRGGRRGGRRGGRRGGGRRGG. In embodiments, an NBP may comprise one or more RGG, RGGR, or RGGGR interspersed throughout its sequence. In embodiments, an NBP contains from 1 to 100 RG, RGG, RGGR, or RGGGR sequences. The RGG, RGGR, and RGGGR may be interspersed throughout the sequence (separated by one or more amino acids) or consecutive. In embodiments, the NBP comprises about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, or about 100 RG, RGG, RGGR, or RGGGR repeats. The RG, RGG, RGGR, and RGGGR repeats may be consecutive or interspersed throughout the sequence. The following article describes exemplary RGG sequences and is incorporated by reference herein in its entirety: Simon et al. Molecular Cell (2019), 75, 66-75.e5.

In embodiments, an NBP comprises an RG domain. An RG domain comprises from about 2 to about 500 repeats of RG (arginine-glycine). In embodiments, an NBP comprises an RGG domain. An RGG domain comprises from about 2 to about 500 repeats of RGG (arginine-glycine-glycine). In embodiments, an NBP comprises an RGGR domain. An RGGR domain comprises from about 2 to about 500 repeats of RGGR (arginine-glycine-glycine-arginine). In embodiments, an NBP comprises an RGGGR domain. An RGGGR domain comprises from about 2 to about 500 repeats of RGG (arginine-glycine-glycine-glycine-arginine). In embodiments, an NBP comprise an RG mix domain. An RG mix domain comprises 2-500 simultaneous repeats of RG, RGG, RGGR, and/or RGGGR. For example, the RG mix domain may comprise RGG, followed by RG, followed by RGGR, followed by RG, followed by RGGGR.

In embodiments, the NBP comprises a K/R basic patch. A K/R basic patch contains from 4-8 consecutive lysines, arginines, or a combination thereof. K/R basic patches form a highly positive and exposed interface which binds to RNA. K/R basic patches are frequently contained in multiple clusters on the same protein.

In embodiments, the NBP comprises a molecular recognition feature (MoRF). In embodiments, the MoRF is up to 25 amino acids long, 50 or more amino acids long, or from 25 to 50 amino acids in length. MoRFs undergo a dynamic disorder-to-order transition upon ligand binding.

In embodiments, the NBP comprises a low complexity (LC) sequence. In embodiments, LC sequences contain up to 100 amino acids and are composed of many repeats of the same amino acid or several amino acid. LC sequences can polymeraize into amyloid-like fibers and undergo reversible phase transition to a hydrogel-like state. Examples of proteins containing LC sequences are FUS and hnRNPA2.

In embodiments, the NBP comprises a RNA recognition motif (RRM). RRMs bind to RNA. Typically, binding is sequence-specific. In embodiments, RRMs comprise from about 75 to about 125 amino acids, for example, about 75, about 80, about 85, about 90, about 95, about 100, about 105, about 110, about 115, about 120, or about 125 amino acids in length. In embodiments, an RRM comprises about 85 amino acids. RRMs typically adopt a β₁α₁β₂β₃α₂β₄ topology forming two alpha-helices against an antiparallel beta sheet, which houses the conserved RNA-binding RNP1 and RNP2 motifs in central β₁ and β₃ strands.

In embodiments, the NBP comprises a double stranded RNA-binding domain (dsRBD). In embodiments, a dsRBD comprises from about 55 to about 80 amino acids, or from about 65 to about 70 amino acids. In embodiments, a dsRBD comprises 68 amino acids. dsRBD typically adopt an αβββα conformation. In embodiments, a dsRBD occurs as a tandem repeats or in combination with other RNA binding domains. There are two subclasses of dsRBDs, type B and type A. Type A has better binding to dsRNA than type B. dsRBDs typically bind in a shape dependent fashion and not sequence specific. However, ADAR2 is a rare example of a dsRBD that exhibits sequence specific binding.

In embodiments, the NBP comprises a K homology domain. In embodiments, the K homology domain comprises from 60 to 80 amino acids. In embodiments, the K homology domain comprises 70 amino acids. There are two types of K homology domains: type I or reverse type II. The type I K homology domain adopts the β₁α₁α₂β₂β′α′topology. The reverse type II K homology domain adopts the α′β′β₁α₁α₂β₂ topology. K homology domains do not use aromatic amino acids for binding and instead use hydrogen bonding. NBP containing K homology domain are difficult to design due to their stringent sequence specificity.

In embodiments, the NBP comprises one or more zinc finger (ZF) domains. In embodiments, the NBP comprises from 1-100 ZF domains, for example, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, or about 100 ZF domains. In embodiments, the zinc finger domain is selected from one of the following subtypes: CCHC (zinc knuckle), CCCH, CCCC (RanBP2), and CCHH. C and H refer to the interspersed cysteine and histidine residues that coordinate the zinc atom. In embodiments, a zinc finger domain comprises from about 20 to about 40 amino acids, for example, about 20, about 22, about 24, about 26, about 28, about 30, about 32, about 34, about 36, about 38, or about 40 amino acids. CCHH ZF domains contain two conserved cysteine and two conserved histidine residues. CCHH ZF domains recognize both structural and sequence specific elements. To this date, there are no engineered versions of CCHH ZF domains. CCHH ZF domains bind both single stranded and double stranded DNA and RNA. CCCC ZFs might not require a specific RNA conformation for binding. Typically, CCCC ZFs recognize short three nucleotide repeats. An engineered version of the CCCC ZF is described in the following reference which is incorporated by reference herein in its entirety: De Franco et al. Sci Rep: 2019: 9, 2484.

In embodiment, the NBP comprises a pentatricopeptide repeat (PPR). PPR contain from 20-50 amino acids, for example, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50 amino acids. In embodiments, a PPR comprises about 35 amino acids. In embodiments, an NBP comprises a PPR that repeats from about 2-30 times within the NBP sequence. In embodiments, an NBP comprises a PPR that repeats from about 10-30 times within the NBP sequence. In embodiments, the PPR may repeat about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 times within the NBP. In embodiments, the PPR repeats at least 10 times. PPR repeats may be consecutive or separated by one or more amino acids. PPR repeats form two antiparallel α-helices. In embodiments, NBPs comprising a PPR form a solenoid structure. In embodiments, NBPs comprising a PPR bind to single stranded RNA, single stranded DNA., or mRNA. In embodiments, NBPs comprising a PPR bind to the 5′ cap of mRNA. In embodiments, NBPs comprising a PPR bind to the 3′ poly A tail of mRNA.

In embodiments, an NBP comprises a Pumilio homology domain (also referred to as Pumillo-like repeat, abbreviated “PUF”). PUF domains contain eight α-helical repeats of a conserved 36 amino acid sequence that forms a concave RNA binding surface. In embodiments, an NBP comprises from 1-8 of the α-helical repeats of a PUF domain, for example, 1, 2, 3, 4, 5, 6, 7, or 8 α-helical repeats. In embodiments, an NBP comprising a PUF binds to a poly A tail. In embodiments, an NBP comprising a PUF binds to mRNA. The following paper describes PUF domains and is incorporated by reference herein in its entirety: Zhao et al. Nucleic Acids Res. 2018 May 18; 46(9): 4771-4782. In embodiments, an NBP comprising a PUF domain binds to the 3′ untranslated region of mRNA.

In embodiments, an NBP comprises a cold shock domain (CSD). CSD contain five antiparallel β-strands that form a β-barrel structure known as an oligosaccharide-/oligonucleotide binding fold. CSD bind to single stranded RNA and single stranded DNA. In embodiments, CSD are comprised of from 60 to 80 amino acids, for example, about 60, about 61, about 62, about 63, about 64, about 65, about 66, about 67, about 68, about 69, about 70, about 71, about 72, about 73, about 74, about 75, about 76, about 77, about 78, about 79, or about 80 amino acids. In embodiments, CSD contain about 70 amino acids. In embodiments, the CSD is a bacterial CSD. In embodiments, bacterial CSD prefer ssDNA to ssRNA by up to ten-fold.

In embodiments, an NBP comprises a helicase domain. Helicases comprise six superfamilies (SFs), including SF1, SF2, SF3, SF4, SF5, and SF6. In embodiments, the helicase domain is a eukaryotic RNA and DNA helicase from the SF1 or SF2 superfamilies. Non-limiting examples of families within the SF1 and SF2 superfamilies include the Upf1-like family, the DEAD-box, DEAH, RIG-I-like, Ski2-like, and NS3 families. In embodiments, the helicase domain is a bacterial or viral helicase from the SF3, SF4, SF5, or SF6 superfamily. ATP binding to a helicase promotes higher affinity of a helicase domain to RNA. ATP hydrolysis promotes conformational changes that cause the helicase to unwind its substrate and/or translocate one nucleotide.

In embodiments, a NBP comprises a La motif. The La motif consists of five α-helices and three β-strands that form a small antiparallel β-sheet against a modified “winged-helix” fold. In embodiments, the La motif binds to 3′-terminal UUU-OH elements on polymerase III transcribed small RNAs. La motifs comprise between 80 and 100 amino acids, for example, about 80, about 81, about 82, about 83, about 84, about 85, about 86, about 87, about 88, about 89, about 90, about 91, about 92, about 93, about 94, about 95, about 96, about 97, about 98, about 99, or about 100 amino acids. In embodiments, a La motif comprises about 90 amino acids.

In embodiments, a NBP comprises a Piwi-Argonautre-Zwille (PAZ) domain. In embodiments, a PAZ domain facilitates binding of small interfering mRNA and/or microRNA guides to mRNA targets. In embodiments, the PAZ domain is from a Dicer protein or an Argonaute protein. PAZ domains display a six-stranded β-barrel topped with two α-helices and flanked on the opposite side by a special appendage containing a β-hairpin and short α-helix.

In embodiments, a NBP comprises a P-element induced Wimpy Testis (PIWI) domain. In embodiments, a PIWI domain facilitates binding of small interfering mRNA and/or microRNA guides to mRNA targets. In embodiments, a PIWI domain is found on an Argonaute protein. The PIWI domain tertiary structure forms an RNase H-like fold consisting of a five-stranded β-sheet flanked by α-helices on both faces.

In embodiments, a NBP comprises a PAZ domain and a PIWI domain.

In embodiments, a NBP comprises a pseudouridine synthase and archaeosine transglycosylase (PUA) domain. PUA domains range from 67-94 amino acids in length, with a β₁α₁β₂β₃β₄β₅α₂β₆ architecture that forms a pseudobarrel encased by two α-helices. In embodiments, an NBP comprising a PUA binds to double stranded RNA.

In embodiments, a NBP comprises a S1 RNA binding domain. In embodiments, an NBP comprising a S1 RNA binding domain interacts with single stranded RNA, double stranded RNA, or mRNA. In embodiments, the S1 RNA binding domain comprises from about 60 to about 80 amino acids, for example, about 60, about 61, about 62, about 63, about 64, about 65, about 66, about 67, about 68, about 69, about 70, about 71, about 72, about 73, about 74, about 75, about 76, about 77, about 78, about 79, or about 80 amino acids. In embodiments, the S1 RNA binding domain comprises about 70 amino acids.

In embodiments, a NBP comprises an Sm RNA binding motif. Sm RNA binding motifs are found in Sm and Like-sm (Lsm) proteins in eukaryotes and archaea and in Hfq proteins in prokaryotes. The Sm motif consists of ˜70 residues with an α₁β₁β₂β₃β₄β₅ topology that forms a curved antiparallel β-sheet. Sm-containing proteins readily multimerize through interactions between strands β₄ and β₅ in two Sm motifs. In embodiments, a NBP comprises 1, 2, 3, 4, 5, 6, 7, 8, 9, or 10 Sm motifs. In embodiments, an NBP comprises two Sm motifs. In embodiments, an Sm binding motif binds to RNA through hydrogen bonding and base stacking interactions.

In embodiments, a NBP comprises a thiouridine synthase and RNA methylase and pseudouridine synthase (THUMP) domain. The THUMP domain is found in many tRNA-modifying enzymes. THUMP domains are found in proximity to RNA-modifying domains and sometimes in proximity to an N-terminal ferredoxin-like domain. THUMP domains display a α₁α₂β₁α₃β₂β₂ topology that forms parallel α-helices flanking a β-sheet. In embodiments, an NBP comprising a THUMP domain binds to tRNA.

In embodiments, a NBP comprises YT521-B homology domain. In embodiments, a YT521-B homology domains comprises from 100-150 amino acids, for example, about 100, about 101, about 102, about 103, about 104, about 105, about 106, about 107, about 108, about 109, about 110, about 111, about 112, about 113, about 114, about 115, about 116, about 117, about 118, about 119, about 120, about 121, about 122, about 123, about 124, about 125, about 126, about 127, about 128, about 129, about 130, about 131, about 132, about 133, about 134, about 135, about 136, about 137, about 138, about 139, about 140, about 141, about 142, about 143, about 144, about 145, about 146, about 147, about 148, about 149, or about 150 amino acids. In embodiments, a NBP comprising a YT521-B homology domain binds to a methylated adenosine.

In some embodiments, the first polypeptide is selected from cluster of differentiation 4 (CD4), the Z-domain of Staphylococcus protein A (SpA Z-domain), low-density lipoprotein receptor (LDLR), albumin binding polypeptide (ABD), coxsackievirus and adenovirus receptor (CAR), fibronectin type III (FN3), poly(A) binding protein (PABP), Z-DNA binding protein 1 (ZBP1), or a fragment or derivative thereof.

In some embodiments, the first polypeptide of the fusion protein comprises cluster of differentiation 4 (CD4), or a fragment or derivative thereof. CD4 binds to lentivirus particles, retrovirus particles, and glycoprotein 120 (gp120) of human immunodeficiency virus.

CD4 (see, e.g., Uniprot Accession No. P01730) is a glycoprotein found on the surface of immune cells such as T helper cells, monocytes, macrophages, and dendritic cells. The amino acid sequence of CD4 from Homo sapiens is (M)NRGVPFRHLLLVLQLALLPAATQGKKVVLGKKGDTVELTCTASQKKSIQFHWKNSN QIKILGNQGSFLTKGPSKLNDRADSRRSLWDQGNFPLIIKNLKIEDSDTYICEVEDQKEEV QLLVFGLTANSDTHLLQGQSLTLTLESPPGSSPSVQCRSPRGKNIQGGKTLSVSQLELQD SGTWTCTVLQNQKKVEFKIDIVVLAFQKASSIVYKKEGEQVEFSFPLAFTVEKLTGSGEL WWQAERASSSKSWITFDLKNKEVSVKRVTQDPKLQMGKKLPLHLTLPQALPQYAGSG NLTLALEAKTGKLHQEVNLVVMRATQLQKNLTCEVWGPTSPKLMLSLKLENKEAKVS KREKAVWVLNPEAGMWQCLLSDSGQVLLESNIKVLPTWSTPVQPMALIVLGGVAGLLL FIGLGIFFCVRCRHRRRQAERMSQIKRLLSEKKTCQCPHRFQKTCSPI (SEQ ID NO: 78).

In some embodiments, the first polypeptide comprises CD4 having an amino acid sequence of SEQ ID NO: 78 and at least one, at least two, at least three, or at least four mutations of amino acids 112, 113, 116, and 117 to glycine, alanine, lysine, arginine, and histidine.

In some embodiments, the first polypeptide comprises the extracellular domain of CD4. In some embodiments, the first polypeptide comprises the extracellular domain of CD4 having an amino acid sequence of SEQ ID NO: 79.

In some embodiments, the first polypeptide comprises a fragment of the extracellular domain of CD4 having an amino acid sequence of SEQ ID NO: 80. In some embodiments, the first polypeptide comprises domain 1 of CD4 having an amino acid sequence of SEQ ID NO: 81. In some embodiments, the first polypeptide comprises the extracellular domain of CD4 or a fragment or derivative thereof (SEQ ID NOs: 79-81) having at least one, at least two, at least three, or at least four mutations at amino acids 88, 89, 92, and 93 to glycine, alanine, lysine, arginine, or histidine.

In some embodiments, the first polypeptide comprising CD4, or a fragment or derivative thereof, comprises an amino acid sequence selected from any one of SEQ ID NOs: 78-81. In some embodiments, the first polypeptide comprising CD4, or a fragment or derivative thereof, comprises the amino acid sequence of any one of SEQ ID NOs: 78-81 with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25 or more amino acid mutations. In some embodiments, the first polypeptide comprising CD4, or fragment thereof, comprises a sequence with at least 90%, at least 95%, at least 97%, or at least 99% identity to any one of SEQ ID Nos: 78-81.

In some embodiments, the first polypeptide comprises protein A, or a derivative or fragment thereof. Protein A (see, e.g., Uniprot Accession No. Q70AB8), binds to the Fc region of most immunoglobulins. In some embodiments, protein A is Staphylococcal protein A. The amino acid sequence of Protein A is:

(SEQ ID NO: 136)
(M)AAQHDEAQQNAFYQVLNMPNLNADQRNGFIQSLKDDPSQSAN
VLGEAKKLNESQAPKADNNFNKEQQNAFYEILNMPNLNEEQRNGF
IQSLKDDPSQSANLLSEAKKLNESQAPKADNKFNKEQQNAFYEIL
HLPNLNEEQRNGFIQSLKDDPSQSANLLAEAKKLNDAQAPKADNK
FNKEQQNAFYEILHLPNLTEEQRNGFIQSLKDDPSVSKEILAEAK
KLNDAQAPKEEDNNKPGKEDGNKPGKEDGN.

In some embodiments, the first polypeptide comprises Protein A having an amino acid sequence of SEQ ID NO: 136 with a mutation of A117G. In some embodiments, the first polypeptide comprises the B domain of Protein A or a fragment or derivative thereof, having the amino acid sequence of SEQ ID NO: 137. In some embodiments, the first polypeptide comprises the B domain of Protein A or a fragment or derivative thereof, having the amino acid sequence of SEQ ID NO: 137 with an amino acid mutation of A2G. In some embodiments, the first polypeptide comprises the C domain of Protein A or a fragment or derivative thereof, having the amino acid sequence of SEQ ID NO: 138. In some embodiments, the first polypeptide comprises the Z domain of Protein A, or a fragment or derivative thereof, having the amino acid sequence of SEQ ID NO:

In some embodiments, the first polypeptide comprising Protein G, or a fragment or derivative thereof, comprises the amino acid sequence of SEQ ID NOs: 136-138 with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25 or more amino acid mutations. In some embodiments, the first polypeptide comprising an albumin binding polypeptide, or fragment or derivative thereof, comprises a sequence with at least 90%, at least 95%, at least 97%, or at least 99% identity to any one of SEQ ID NOs: 136-138.

In some embodiments, the first polypeptide comprises protein G, or a derivative or fragment thereof. Protein G (see, e.g., Uniprot Accession No. P19909), binds to the Fc region of most immunoglobulins. The amino acid sequence of Protein G is:

(SEQ ID NO: 154)
(M)EKEKKVKYFLRKSAFGLASVSAAFLVGSTVFAVDSPIEDTPI
IRNGGELTNLLGNSETTLALRNEESATADLTAAAVADTVAAAAAE
NAGAAAWEAAAAADALAKAKADALKEFNKYGVSDYYKNLINNAKT
VEGVKDLQAQVVESAKKARISEATDGLSDFLKSQTPAEDTVKSIE
LAEAKVLANRELDKYGVSDYHKNLINNAKTVEGVKDLQAQVVESA
KKARISEATDGLSDFLKSQTPAEDTVKSIELAEAKVLANRELDKY
GVSDYYKNLINNAKTVEGVKALIDEILAALPKTDTYKLILNGKTL
KGETTTEAVDAATAEKVFKQYANDNGVDGEWTYDDATKTFTVTEK
PEVIDASELTPAVTTYKLVINGKTLKGETTTEAVDAATAEKVFKQ
YANDNGVDGEWTYDDATKTFTVTEKPEVIDASELTPAVTTYKLVI
NGKTLKGETTTKAVDAETAEKAFKQYANDNGVDGVWTYDDATKTF
TVTEMVTEVPGDAPTEPEKPEASIPLVPLTPATPIAKDDAKKDD
TKKEDAKKPEAKKEDAKKAETLPTTGEGSNPFFTAAALAVMAGAG
ALAVASKRKED.

In some embodiments, the first polypeptide comprises the G domain of Protein G or a fragment or derivative thereof, having the amino acid sequence of SEQ ID NO: 155. In some embodiments, the first polypeptide comprises a fragment of Protein G, having the amino acid sequence of SEQ ID NO: 156.

In some embodiments, the first polypeptide comprising Protein G, or a fragment or derivative thereof, comprises the amino acid sequence of SEQ ID NOs: 154-156 with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25 or more amino acid mutations. In some embodiments, the first polypeptide comprising an albumin binding polypeptide, or fragment or derivative thereof, comprises a sequence with at least 90%, at least 95%, at least 97%, or at least 99% identity to any one of SEQ ID NOs: 154-156.

In some embodiments, the first polypeptide binds to a kappa light chain. In some embodiments, the kappa light chain is part of an antibody or antigen-binding portion of a monoclonal antibody thereof. In some embodiments, the first polypeptide comprises protein L. Protein L binds to antibodies or antibody binding fragments thereof through interactions with the kappa light chain. The amino acid sequence of Protein L is:

(SEQ ID NO: 139)
(M)KINKKLLMAALAGAIVVGGGANAYAAEEDNTDNNLSMDEISD
AYFDYHGDVSDSVDPVEEEIDEALAKALAEAKETAKKHIDSLNHL
SETAKKLAKNDIDSATTINAINDIVARADVMERKTAEKEEAEKLA
AAKETAKKHIDELKHLADKTKELAKRDIDSATTINAINDIVARAD
VMERKTAEKEEAEKLAAAKETAKKHIDELKHLADKTKELAKRDID
SATTIDAINDIVARADVMERKLSEKETPEPEEEVTIKANLIFADG
STQNAEFKGTFAKAVSDAYAYADALKKDNGEYTVDVADKGLTLNI
KFAGKKEKPEEPKEEVTIKVNLIFADGKTQTAEFKGTFEEATAKA
YAYADLLAKENGEYTADLEDGGNTINIKFAGKETPETPEEPKEEV
TIKVNLIFADGKIQTAEFKGTFEEATAKAYAYANLLAKENGEYTA
DLEDGGNTINIKFAGKETPETPEEPKEEVTIKVNLIFADGKTQTA
EFKGTFEEATAEAYRYADLLAKVNGEYTADLEDGGYTINIKFAGK
EQPGENPGITIDEWLLKNAKEEAIKELKEAGITSDLYFSLINKAK
TVEGVEALKNEILKAHAGEETPELKDGYATYEEAEAAAKEALKND
DVNNAYEIVQGADGRYYYVLKIEVADEEEPGEDTPEVQEGYATYE
EAEAAAKEALKEDKVNNAYEVVQGADGRYYYVLKIEDKEDEQPGE
EPGENPGITIDEWLLKNAKEDAIKELKEAGISSDIYFDAINKAKT
VEGVEALKNEILKAHAEKPGENPGITIDEWLLKNAKEAAIKELKE
AGITAEYLFNLINKAKTVEGVESLKNEILKAHAEKPGENPGITID
EWLLKNAKEDAIKELKEAGITSDIYFDAINKAKTIEGVEALKNEI
LKAHKKDEEPGKKPGEDKKPEDKKPGEDKKPEDKKPGEDKKPEDK
KPGKTDKDSPNKKKKAKLPKAGSEAEILTLAAAALSTAAGAYVSL
KKRK.

In some embodiments, the first polypeptide comprises a fragment of Protein L having an amino acid sequence of SEQ ID NO: 140.

In some embodiments, the first polypeptide comprising Protein L, or a fragment or derivative thereof, comprises the amino acid sequence of SEQ ID NOs: 139-140 with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25 or more amino acid mutations. In some embodiments, the first polypeptide comprising an albumin binding polypeptide, or fragment or derivative thereof, comprises a sequence with at least 90%, at least 95%, at least 97%, or at least 99% identity to any one of SEQ ID NOs: 139-140.

In some embodiments, a first polypeptide of a purification matrix provided herein comprises the LDLR, or a fragment or derivative thereof. The low-density lipoprotein receptor LDLR (see, e.g., Uniprot Accession No. P01130) is a cell-surface receptor that mediates the endocytosis of cholesterol rich low-density lipoproteins. The amino acid sequence of LDLR from Homo sapiens is (M)GPWGWKLRWTVALLLAAAGTAVGDRCERNEFQCQDGKCISYKWVCDGSAECQDG SDESQETCLSVTCKSGDFSCGGRVNRCIPQFWRCDGQVDCDNGSDEQGCPPKTCSQDEF RCHDGKCISRQFVCDSDRDCLDGSDEASCPVLTCGPASFQCNSSTCIPQLWACDNDPDC EDGSDEWPQRCRGLYVFQGDSSPCSAFEFHCLSGECIHSSWRCDGGPDCKDKSDEENCA VATCRPDEFQCSDGNCIHGSRQCDREYDCKDMSDEVGCVNVTLCEGPNKFKCHSGECI TLDKVCNMARDCRDWSDEPIKECGTNECLDNNGGCSHVCNDLKIGYECLCPDGFQLVA QRRCEDIDECQDPDTCSQLCVNLEGGYKCQCEEGFQLDPHTKACKAVGSIAYLFFTNRH EVRKMTLDRSEYTSLIPNLRNVVALDTEVASNRIYWSDLSQRMICSTQLDRAHGVSSYD TVISRDIQAPDGLAVDWIHSNIYWTDSVLGTVSVADTKGVKRKTLFRENGSKPRAIVVD PVHGFMYWTDWGTPAKIKKGGLNGVDIYSLVTENIQWPNGITLDLLSGRLYWVDSKLH SISSIDVNGGNRKTILEDEKRLAHPFSLAVFEDK VFWTDIINEAIFSANRLTGSDVNLLAE NLLSPEDMVLFHNLTQPRGVNWCERTTLSNGGCQYLCLPAPQINPHSPKFTCACPDGML LARDMRSCLTEAEAAVATQETSTVRLKVSSTAVRTQHTTTRPVPDTSRLPGATPGLTTV EIVTMSHQALGDVAGRGNEKKPSSVRALSIVLPIVLLVFLCLGVFLLWKNWRLKNINSIN FDNPVYQKTTEDEVHICHNQDGYSYPSRQMVSLEDDVA (SEQ ID NO: 126).

In some embodiments, the first polypeptide comprises the extracellular domain of LDLR having an amino acid sequence of SEQ ID NO: 127. In some embodiments, the first polypeptide comprises a fragment of the extracellular domain of LDLR having an amino acid sequence of SEQ ID NO: 130. In some embodiments, the first polypeptide comprises the CR2 domain of LDLR having an amino acid sequence of SEQ ID NO: 128. In some embodiments, the first polypeptide comprises the CR3 domain of LDLR having an amino acid sequence of SEQ ID NO: 129.

In some embodiments, the first polypeptide comprising LDLR, or a fragment or derivative thereof, comprises an amino acid sequence selected from any one of SEQ ID NOs: 126-129. In some embodiments, the first polypeptide comprising LDLR, or a fragment or derivative thereof, comprises the amino acid sequence of any one of SEQ ID NOs: 126-129 with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25 or more amino acid mutations. In some embodiments, the first polypeptide comprising LDLR, or fragment thereof, comprises a sequence with at least 90%, at least 95%, at least 97%, or at least 99% identity to any one of SEQ ID Nos: 126-129.

In some embodiments, the first polypeptide binds an albumin, or derivative or fusion thereof. In some embodiments, the albumin is human serum albumin (HSA), bovine serum albumin (BSA), or ovalbumin. In some embodiments, the first polypeptide that binds an albumin comprises albumin-binding polypeptide (ABP), or a fragment or derivative thereof. In some embodiments, the albumin-binding polypeptide comprises an amino acid sequence of SEQ ID NO: 135. In some embodiments, the first polypeptide comprising an albumin-binding polypeptide, or fragment or derivative thereof, comprises the amino acid sequence of SEQ ID NO: 135 with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25 or more amino acid mutations. In some embodiments, the first polypeptide comprising an albumin-binding polypeptide, or fragment or derivative thereof, comprises a sequence with at least 90%, at least 95%, at least 97%, or at least 99% identity to SEQ ID NO: 135.

In some embodiments, the first polypeptide is a coxsackievirus and adenovirus receptor (CAR), or fragment or derivative thereof. The coxsackievirus and adenovirus receptor (see, e.g., Uniprot Accession No. P78310), which is expressed on heart, brain epithelial, and endothelial cells, binds to the fiber protein of the adenovirus capsid. The amino acid sequence of the coxsackievirus and adenovirus receptor from Homo sapiens is:

(SEQ ID NO: 117)
(M)ALLLCFVLLCGVVDFARSLSITTPEEMIEKAKGETAYLPCKF
TLSPEDQGPLDIEWLISPADNQKVDQVIILYSGDKIYDDYYPDLK
GRVHFTSNDLKSGDASINVTNLQLSDIGTYQCKVKKAPGVANKK
IHLVVLVKPSGARCYVDGSEEIGSDFKIKCEPKEGSLPLQYEWQ
KLSDSQKMPTSWLAEMTSSVISVKNASSEYSGTYSCTVRNRVGSD
QCLLRLNVVPPSNKAGLIAGAIIGTLLALALIGLIIFCCRKKRRE
EKYEKEVHHDIREDVPPPKSRTSTARSYIGSNHSSLGSMSPSNME
GYSKTQYNQVPSEDFERTPQSPTLPPAKVAAPNLSRMGAIPVMIP
AQSKDGSIV.

In some embodiments, a first polypeptide of a purification matrix provided herein comprises the coxsackievirus and adenovirus receptor, or a fragment or derivative thereof.

In some embodiments, the first polypeptide comprising a coxsackievirus and adenovirus receptor, or fragment thereof, comprises an amino acid sequence selected from any one of SEQ ID NOs: 117-125, 24 and 25. In some embodiments, the first polypeptide comprising a coxsackievirus and adenovirus receptor, or fragment thereof, comprises the amino acid sequence of any one of SEQ ID NOs: 117-125, 24 and 25 with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25 or more amino acid mutations. In some embodiments, the first polypeptide comprising a coxsackievirus and adenovirus receptor, or fragment thereof, comprises a sequence with at least 90%, at least 95%, at least 97%, or at least 99% identity to any one of SEQ ID NOs: 117-125, 24 and 25.

In some embodiments, the first polypeptide comprises the extracellular domain of the coxsackievirus and adenovirus receptor, or a fragment thereof. In some embodiments, the first polypeptide comprising the extracellular domain of the coxsackievirus and adenovirus receptor has an amino acid sequence of SEQ ID NO: 118 or 119. In some embodiments, the first polypeptide comprises domain 1 of the coxsackievirus and adenovirus receptor, or a fragment thereof. In some embodiments, the first polypeptide comprising domain 1 of the coxsackievirus and adenovirus receptor has an amino acid sequence of SEQ ID NO: 120. In some embodiments, the first polypeptide comprises domain 2 of the coxsackievirus and adenovirus receptor, or a fragment thereof. In some embodiments, the first polypeptide comprising domain 2 of the coxsackievirus and adenovirus receptor has an amino acid sequence of SEQ ID NO: 121. In some embodiments, the first polypeptide comprises isoform 3 of the coxsackievirus and adenovirus receptor, or a fragment thereof. In some embodiments, the first polypeptide comprising isoform 3 of the coxsackievirus and adenovirus receptor has an amino acid sequence of SEQ ID NO: 122. In some embodiments, the first polypeptide comprises isoform 4 of the coxsackievirus and adenovirus receptor, or a fragment thereof. In some embodiments, the first polypeptide comprising isoform 4 of the coxsackievirus and adenovirus receptor has an amino acid sequence of SEQ ID NO: 123. In some embodiments, the first polypeptide comprises isoform 5 of the coxsackievirus and adenovirus receptor, or a fragment thereof. In some embodiments, the first polypeptide comprising isoform 5 of the coxsackievirus and adenovirus receptor has an amino acid sequence of SEQ ID NO: 124. In some embodiments, the first polypeptide comprises isoform 7 of the coxsackievirus and adenovirus receptor, or a fragment thereof. In some embodiments, the first polypeptide comprising isoform 7 of the coxsackievirus and adenovirus receptor has an amino acid sequence of SEQ ID NO: 125.

In some embodiments, the first polypeptide comprises an amino acid sequence of M(RAIVFRVQWLRRYFVNGSRSGGG)ₙ, where n is an integer from 1 to 8 (SEQ ID NO: 24), for example, n is 1, 2, 3, 4, 5, 6, 7, or 8. In some embodiments, the first polypeptide comprises an amino acid sequence of (RAIVFRVQWLRRYFVNGSRSGGG)ₙ, wherein n is an integer from 1 to 8 (SEQ ID NO: 25), for example, 1, 2, 3, 4, 5, 6, 7, or 8.

In some embodiments, the first polypeptide comprises mRNA decay activator protein ZFP36L2 (Tis11d), or a fragment or derivative thereof. Tis11d G (see, e.g., Uniprot Accession No. P47974) binds to an adenosine and uridine rich element (ARE). The amino acid sequence of Tis11d is: (M)STTLLSAFYDVDFLCKTEKSLANLNLNNMLDKKAVGTPVAAAPSSGFAPGFLRRHS ASNLHALAHPAPSPGSCSPKFPGAANGSSCGSAAAGGPTSYGTLKEPSGGGGTALLNKE NKFRDRSFSENGDRSQHLLHLQQQQKGGGGSQINSTRYKTELCRPFEESGTCKYGEKCQ FAHGFHELRSLTRHPKYKTELCRTFHTIGFCPYGPRCHFIHNADERRPAPSGGASGDLRA FGTRDALHLGFPREPRPKLHHSLSFSGFPSGHHQPPGGLESPLLLDSPTSRTPPPPSCSSAS SCSSSASSCSSASAASTPSGAPTCCASAAAAAAAALLYGTGGAEDLLAPGAPCAACSSAS CANNAFAFGPELSSLITPLAIQTHNFAAVAAAAYYRSQQQQQQQGLAPPAQPPAPPSATL PAGAAAPPSPPFSFQLPRRLSDSPVFDAPPSPPDSLSDRDSYLSGSLSSGSLSGSESPSLDPG RRLPIFSRLSISDD (SEQ ID NO: 169). The methionine enclosed by parenthesis in the aforementioned sequence (M) or in any other sequences described herein is an initiator methionine. The presence of an initiator methionine is optional.

In some embodiments, the first polypeptide comprises a Tis11d fragment having an amino acid sequence of SEQ ID NO: 170. In some embodiments, the first polypeptide comprises the RNA binding domain of Tis11d having an amino acid sequence of SEQ ID NO: 171.

In some embodiments, the first polypeptide comprises Tis11d having an amino acid sequence of SEQ ID NO: 169 with at least one mutation selected from E195D, E195H, E195G, E195A, E195R, and E195K. In some embodiments, the first polypeptide comprises a Tis11d fragment having an amino acid sequence of SEQ ID NO: 170 with a mutation selected from at least one of E46D, E46H, E46G, E46A, E46R, and E46K. In some embodiments, the first polypeptide comprises the RNA binding domain of Tis11d having an amino acid sequence of SEQ ID NO: 171 with at least one mutation selected from E27D, E27H, E27G, E27A, E27R, and E27K.

In some embodiments, a first polypeptide comprising Tis11d, or a fragment or derivative thereof, comprises the amino acid sequence of SEQ ID NOs: 169-171 with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25 or more amino acid mutations. In some embodiments, the first polypeptide comprising Tis11d, or a fragment or derivative thereof, comprises a sequence with at least 90%, at least 95%, at least 97%, or at least 99% identity to any one of SEQ ID NOs: 169-171.

In some embodiments, the first polypeptide comprises eukaryotic translation initiation factor 4E (eIF4E), or a fragment or derivative thereof. eIF4E (see, e.g., Uniprot Accession No. P06730) binds to the mRNA cap. The amino acid sequence of eIF4E is:

(SEQ ID NO: 172)
(M)ATVEPETTPTPNPPTTEEEKTESNQEVANPEHYIKHPLQNRW
ALWFFKNDKSKTWQANLRLISKFDTVEDFWALYNHIQLSSNLMPG
CDYSLFKDGIEPMWEDEKNKRGGRWLITLNKQQRRSDLDRFWLET
LLCLIGESFDDYSDDVCGAVVNVRAKGDKIAIWTTECENREAVTH
IGRVYKERLGLPPKIVIGYQSHADTATKSGSTTKNRFVV.

In some embodiments, the first polypeptide comprises an eIF4E fragment having an amino acid sequence of SEQ ID NO: 173.

In some embodiments, a first polypeptide comprising eIF4E, or a fragment or derivative thereof, comprises the amino acid sequence of SEQ ID NOs: 172-173 with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25 or more amino acid mutations. In some embodiments, the first polypeptide comprising eIF4E, or fragment or derivative thereof, comprises a sequence with at least 90%, at least 95%, at least 97%, or at least 99% identity to any one of SEQ ID NOs: 172-173.

In some embodiments, the first polypeptide comprises poly(A)-binding protein (PABP), or a fragment or derivative thereof. PABP (see, e.g., Uniprot Accession No. P11940) binds to the poly(A) tail of mRNA. The amino acid sequence of PABP is:

(SEQ ID NO: 174)
(M)NPSAPSYPMASLYVGDLHPDVTEAMLYEKFSPAGPILSIRV
CRDMITRRSLGYAYVNFQQPADAERALDTMNFDVIKGKPVRIMWS
QRDPSLRKSGVGNIFIKNLDKSIDNKALYDTFSAFGNILSCKVVC
DENGSKGYGFVHFETQEAAERAIEKMNGMLLNDRKVFVGRFKSRK
EREAELGARAKEFTNVYIKNFGEDMDDERLKDLFGKFGPALSVKV
MTDESGKSKGFGFVSFERHEDAQKAVDEMNGKELNGKQIYVGRAQ
KKVERQTELKRKFEQMKQDRITRYQGVNLYVKNLDDGIDDERLRK
EFSPFGTITSAKVMMEGGRSKGFGFVCFSSPEEATKAVTEMNGRI
VATKPLYVALAQRKEERQAHLTNQYMQRMASVRAVPNPVINPYQP
APPSGYFMAAIPQTQNRAAYYPPSQIAQLRPSPRWTAQGARPHPF
QNMPGAIRPAAPRPPFSTMRPASSQVPRVMSTQRVANTSTQTMGP
RPAAAAAAATPAVRTVPQYKYAAGVRNPQQHLNAQPQVTMQQPAV
HVQGQEPLTASMLASAPPQEQKQMLGERLFPLIQAMHPTLAGKIT
GMLLEIDNSELLHMLESPESLRSKVDEAVAVLQAHQAKEAAQKAV
NSATGVPTV.

In some embodiments, the first polypeptide comprises a PABP fragment having an amino acid sequence of SEQ ID NO: 175. In some embodiments, the first polypeptide comprises the RNA recognition motif (RRM) 1 domain of PABP having an amino acid sequence of SEQ ID NO: 176. In some embodiments, the first polypeptide comprises the RRM2 domain of PABP having an amino acid sequence of SEQ ID NO: 177. In some embodiments, the first polypeptide comprises the RRM3 domain of PABP having an amino acid sequence of SEQ ID NO: 178. In some embodiments, the first polypeptide comprises the RRM4 domain of PABP having an amino acid sequence of SEQ ID NO: 179.

In some embodiments, a first polypeptide comprising PABP, or a fragment or derivative thereof, comprises the amino acid sequence of any one of SEQ ID NOs: 174-179 with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25 or more amino acid mutations. In some embodiments, the first polypeptide comprising PABP, or fragment or derivative thereof, comprises a sequence with at least 90%, at least 95%, at least 97%, or at least 99% identity to any one of SEQ ID NOs: 174-179.

In some embodiments, the first polypeptide comprises Z-DNA binding protein 1 (ZBP1), or a fragment or derivative thereof. ZBP1 (see, e.g., Uniprot Accession No. Q9H171) binds to double stranded DNA. The amino acid sequence of ZBP1 is:

(SEQ ID NO: 181)
MAQAPADPGREGHLEQRILQVLTEAGSPVKLAQLVKECQAPKREL
NQVLYRMKKELKVSLTSPATWCLGGTDPEGEGPAELALSSPAERP
QQHAATIPETPGPQFSQQREEDIYRFLKDNGPQRALVIAQALGMR
TAKDVNRDLYRMKSRHLLDMDEQSKAWTIYRPEDSGRRAKSASII
YQHNPINMICQNGPNSWISIANSEAIQIGHGNIITRQTVSREDGS
AGPRHLPSMAPGDSSTWGTLVDPWGPQDIHMEQSILRRVQLGHS
NEMRLHGVPSEGPAHIPPGSPPVSATAAGPEASFEARIPSPGTHP
EGEAAQRIHMKSCFLEDATIGNSNKMSISPGVAGPGGVAGSGEGE
PGEDAGRRPADTQSRSHFPRDIGQPITPSHSKLTPKLETMTLGNR
SHKAAEGSHYVDEASHEGSWWGGGI.

In some embodiments, the first polypeptide comprises Z-binding domain 1 of ZBP1 having an amino acid sequence of SEQ ID NO: 182. In some embodiments, the first polypeptide comprises Z-binding domain 2 of ZBP1 having an amino acid sequence of SEQ ID NO: 183.

In some embodiments, a first polypeptide comprising ZBP1, or a fragment or derivative thereof, comprises the amino acid sequence of any one of SEQ ID NOs: 181-183 with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25 or more amino acid mutations. In some embodiments, the first polypeptide comprising ZBP1, or fragment or derivative thereof, comprises a sequence with at least 90%, at least 95%, at least 97%, or at least 99% identity to any one of SEQ ID NOs: 181-183.

In some embodiments, the first polypeptide comprises PUM-HD domain-containing protein (PUM-HD), or a fragment or derivative thereof. PUM-HD (see, e.g., Uniprot Accession No. B2BXX4) binds to mRNA. The amino acid sequence of PUM-HD is:

(SEQ ID NO: 184)
(M)HRGNEDLSFGDDYEKEIGLLLGEQQRRQEEADEIEKELNLYR
SGSAPPTVDGSVNAAGGLFNGGGRGPFMEFGGGNKGNGFGGDDDE
LRKDPAYLSYYYANMKLNPRLPPPLMSREDLRVAQRLKGSSNVLG
GVGDRRNVNESRSLFSMPPGFDQMNEFEAEKTNASSSEWDANGLI
GLPGLGLGGKQKSFADIFQPDMGHPVSQQPSRPASRNAFDENVDS
TNNQSPSASQGIGAPPPYSYAAVLGSSLSRNGTPDPQAVARVPSP
CLTPIGSGRVSSNDKRNTSNQSPFNGVTSGLNESSDLVNALSGMN
LSGSGGLDDRGQAEQDVEKVRNYMFGFQGGHNEVSQHVFPNKSDQ
AQKATGSLRNLHMRGSQGSAYNGGGLANPYQHLDSPNYCLNNYAL
NPAVASVMANQLGNSNFSPMYDNYSAASALGFSGMDSRLHGGGFE
SRNLGRSNRMMGGGGLQSHMADPMYHQYGRYSENVDALDLLNDPA
MDRSFMGNSYMNMLELQRAYLGAQKSQYGVPYKSGSPNSHSYYGS
PTFGSNMSYPGSPLAHHAMQNSLMSPCSPMRRGEVNMRYPSATRN
YSGGVMGSWHMDASLDEGFGSSLLEEFKSNKTRGFELSEIAGHVV
EFSADQYGSRFIQQKLETATTDEKNMVYEEIMPHALALMTDVFGN
YVIQKFFEHGLPPQRRELGDKLFENVLPLSLQMYGCRVIQKAIEV
VDLDQKIKMVKELDGHVMRCVRDQNGNHVVQKCIECVPEENIEFI
ISTFFGHVVSLSTHPYGCRVIQRVLEHCHDPDTQSKVMEEILSTV
SMLAQDQYGNYVVQHVLEHGKPDERTVIIKELAGKIVQMSQQKFA
SNVVEKCLTFGGPEERELLVNEMLGTTDENEPLQAMMKDQFANYV
VQKVLETCDDQQRELILTRIKVHLNALKKYTYGKHVVARIEKLV
AAGGMHMFLLFPLGLKEENGFAVPNPASDVVRPQVLYSLTRVDGS
AIAF.

In some embodiments, a first polypeptide comprising PUM-HD, or a fragment or derivative thereof, comprises the amino acid sequence of SEQ ID NO: 184 with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25 or more amino acid mutations. In some embodiments, the first polypeptide comprising PUM-HD, or fragment or derivative thereof, comprises a sequence with at least 90%, at least 95%, at least 97%, or at least 99% identity to SEQ ID NO: 184.

In some embodiments, the first polypeptide is a fibronectin (FN). In some embodiments, the fibronectin is a fibronectin type III (FN3) repeat. FN3 repeats are both the largest and the most common of the fibronectin subdomains. Domains homologous to FN3 repeats have been found in various animal protein families including other extracellular-matrix molecules, cell-surface receptors, enzymes, and muscle protein. FN3 domains have a conserved beta sandwich fold with one beta sheet containing four strands and the other sheet containing three strands. The amino acid sequence of an exemplary fibronectin (see e.g., UniProt Accession No. P02751) is:

(SEQ ID NO: 26)
MLRGPGPGLLLLAVQCLGTAVPSTGASKSKRQAQQMVQPQSPVAV
SQSKPGCYDNGKHYQINQQWERTYLGNALVCTCYGGSRGFNCESK
PEAEETCFDKYTGNTYRVGDTYERPKDSMIWDCTCIGAGRGRISC
TIANRCHEGGQSYKIGDTWRRPHETGGYMLECVCLGNGKGEWTCK
PIAEKCFDHAAGTSYVVGETWEKPYQGWMMVDCTCLGEGSGRITC
TSRNRCNDQDTRTSYRIGDTWSKKDNRGNLLQCICTGNGRGEWKC
ERHTSVQTTSSGSGPFTDVRAAVYQPQPHPQPPPYGHCVTDSGVV
YSVGMQWLKTQGNKQMLCTCLGNGVSCQETAVTQTYGGNSNGEPC
VLPFTYNGRTFYSCTTEGRQDGHLWCSTTSNYEQDQKYSFCTDHT
VLVQTRGGNSNGALCHFPFLYNNHNYTDCTSEGRRDNMKWCGTTQ
NYDADQKFGFCPMAAHEEICTTNEGVMYRIGDQWDKQHDMGHMMR
CTCVGNGRGEWTCIAYSQLRDQCIVDDITYNVNDTFHKRHEEGHM
LNCTCFGQGRGRWKCDPVDQCQDSETGTFYQIGDSWEKYVHGVRY
QCYCYGRGIGEWHCQPLQTYPSSSGPVEVFITETPSQPNSHPIQW
NAPQPSHISKYILRWRPKNSVGRWKEATIPGHLNSYTIKGLKPGV
VYEGQLISIQQYGHQEVTRFDFTTTSTSTPVTSNTVTGETTPFSP
LVATSESVTEITASSFVVSWVSASDTVSGFRVEYELSEEGDEPQY
LDLPSTATSVNIPDLLPGRKYIVNVYQISEDGEQSLILSTSQTTA
PDAPPDTTVDQVDDTSIVVRWSRPQAPITGYRIVYSPSVEGSSTE
LNLPETANSVTLSDLQPGVQYNITIYAVEENQESTPVVIQQETT
GTPRSDTVPSPRDLQFVEVTDVKVTIMWTPPESAVTGYRVDVIPV
NLPGEHGQRLPISRNTFAEVTGLSPGVTYYFKVFAVSHGRESKPL
TAQQTTKLDAPTNLQFVNETDSTVLVRWTPPRAQITGYRLTVGLT
RRGQPRQYNVGPSVSKYPLRNLQPASEYTVSLVAIKGNQESPKAT
GVFTTLQPGSSIPPYNTEVTETTIVITWTPAPRIGFKLGVRPSQG
GEAPREVTSDSGSIVVSGLTPGVEYVYTIQVLRDGQERDAPIVNK
VVTPLSPPTNLHLEANPDTGVLTVSWERSTTPDITGYRITTTPTN
GQQGNSLEEVVHADQSSCTFDNLSPGLEYNVSVYTVKDDKESVPI
SDTIIPEVPQLTDLSFVDITDSSIGLRWTPLNSSTIIGYRITVVA
AGEGIPIFEDFVDSSVGYYTVTGLEPGIDYDISVITLINGGESAP
TTLTQQTAVPPPTDLRFTNIGPDTMRVTWAPPPSIDLTNFLVRYS
PVKNEEDVAELSISPSDNAVVLTNLLPGTEYVVSVSSVYEQHEST
PLRGRQKTGLDSPTGIDFSDITANSFTVHWIAPRATITGYRIRHH
PEHFSGRPREDRVPHSRNSITLTNLTPGTEYVVSIVALNGREESP
LLIGQQSTVSDVPRDLEVVAATPTSLLISWDAPAVTVRYYRITYG
ETGGNSPVQEFTVPGSKSTATISGLKPGVDYTITVYAVTGRGDSP
ASSKPISINYRTEIDKPSQMQVTDVQDNSISVKWLPSSSPVTGYR
VTTTPKNGPGPTKTKTAGPDQTEMTIEGLQPTVEYVVSVYAQNPS
GESQPLVQTAVTNIDRPKGLAFTDVDVDSIKIAWESPQGQVSRYR
VTYSSPEDGIHELFPAPDGEEDTAELQGLRPGSEYTVSVVALHDD
MESQPLIGTQSTAIPAPTDLKFTQVTPTSLSAQWTPPNVQLTGYR
VRVTPKEKTGPMKEINLAPDSSSVVVSGLMVATKYEVSVYALKDT
LTSRPAQGVVTTLENVSPPRRARVTDATETTITISWRTKTETITG
FQVDAVPANGQTPIQRTIKPDVRSYTITGLQPGTDYKIYLYTLND
NARSSPVVIDASTAIDAPSNLRFLATTPNSLLVSWQPPRARITGY
IIKYEKPGSPPREVVPRPRPGVTEATITGLEPGTEYTIYVIALKN
NQKSEPLIGRKKTDELPQLVTLPHPNLHGPEILDVPSTVQKTPFV
THPGYDTGNGIQLPGTSGQQPSVGQQMIFEEHGFRRTTPPTTATP
IRHRPRPYPPNVGEEIQIGHIPREDVDYHLYPHGPGLNPNASTGQ
EALSQTTISWAPFQDTSEYIISCHPVGTDEEPLQFRVPGTSTSAT
LTGLTRGATYNVIVEALKDQQRHKVREEVVTVGNSVNEGLNQPTD
DSCFDPYTVSHYAVGDEWERMSESGFKLLCQCLGFGSGHFRCDSS
RWCHDNGVNYKIGEKWDRQGENGQMMSCTCLGNGKGEFKCDPHEA
TCYDDGKTYHVGEQWQKEYLGAICSCTCFGGQRGWRCDNCRRPGG
EPSPEGTTGQSYNQYSQRYHQRTNTNVNCPIECFMPLDVQADRED
SRE.

In some embodiments, the first polypeptide comprising fibronectin, or a fragment or derivative thereof, has the amino acid sequence of SEQ ID NO: 26 with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25 or more amino acid mutations. In some embodiments, the first polypeptide comprising fibronectin, or fragment or derivative thereof, comprises a sequence with at least 90%, at least 95%, at least 97%, or at least 99% identity to SEQ ID NO: 26.

In some embodiments, the first polypeptide is an adeno-associated virus receptor (AAVR), or a fragment or derivative thereof. The AAVR, also named KIAA0319L (see, e.g., Uniprot Accession No. Q8IZA0), is a 150 kDa glycoprotein which binds to the capsid of multiple AAV serotypes, including AAV1, AAV2, AAV3B, AAV5, AAV6, AAV8, and AAV9. The following references describe the AAVR and are incorporated by reference herein in their entirety: Zhang et al. Nat Microbiol. 2019 April; 4(4):675-682; Pillay et al. Nature. 2016 Feb. 4; 530(7588): 108-12; Zhang et al. Nat Commun. 2019 Aug. 21; 10(1):3760; Summerford et al. Mol Ther. 2016 April; 24(4):663-6.

The ectodomain of the AAVR comprises a motif at the N-terminus with eight cysteines (MANEC) and five immunoglobulin domains known as polycystic kidney disease (PKD) domains. AAV particles bind to the PKD domains to facilitate transduction. Thus, in some embodiments, an AAVR fragment or derivative thereof comprises one, two, three, four, or five PKD domains, or fragments or derivatives thereof. In some embodiments, the AAVR is the human AAVR (SEQ ID NO: 35), mouse AAVR (SEQ ID NO: 36), or orangutan AAVR (SEQ ID NO: 42), or a fragment or derivative thereof. In some embodiments, the AAVR comprises a sequence with at least 90%, at least 95%, at least 97%, or at least 99% identity to any one of SEQ ID NO: 35, 36, or 42. Unless otherwise indicated, sequence identity is determined using the National Center for Biotechnology Information (NCBI)'s Basic Local Alignment Search Tool (BLAST®), available at blast.ncbi.nlm.nih.gov/Blast.cgi. In some embodiments, the sequence identity is calculated over the entire length of the compared sequences. In some embodiments, the sequence identity is calculated over a 20-amino acid, 50-amino acid, 75-amino acid, 100-amino acid, 250-amino acid, 500-amino acid, 750-amino acid, or 1000-amino acid fragment of each compared sequence.

In some embodiments, the AAVR fragment or derivative thereof comprises an ectodomain of AAVR or a fragment or derivative thereof. In some embodiments, the AAVR comprises the amino acid sequence of SEQ ID NO: 33 or SEQ ID NO: 34, or a sequence with at least about 70%, about 75%, about 80%, about 85%, about 90%, about 95%, about 96%, about 97%, about 98%, about 99% identity thereto. In some embodiments, the AAV-binding polypeptide comprises the sequence of SEQ ID NO: 33 or SEQ ID NO: 34 with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25 or more amino acid mutations.

In some embodiments, the AAVR, or fragment or derivative thereof, comprises one or more PKDs, such as two, three, four, five, or more PKDs. In some embodiments, the PKDs are each individually selected from the PKDs listed in Table 3. For example, in some embodiments, the PKDs are each individually selected from SEQ ID NO: 28-32, 37-41, and 43-47. In some embodiments, an AAVR, or fragment or derivative thereof, comprises multiple PKDs, and the PKDs are connected to one another by a linker. Non-limiting examples of linkers are described throughout this disclosure. In some embodiments, an AAVR, or fragment or derivative thereof comprises multiple PKD domains, wherein each PKD domain has the same or substantially the same sequence. In some embodiments, an AAVR, or fragment or derivative thereof, comprises multiple PKD domains, wherein each PKD has a different sequence.

TABLE 3
Amino Acid Sequences of PKDs

| PKD | SEQ ID NO. |
|---|---|
| Human PKD1 | 28 |
| Human PKD2 | 29 |
| Human PKD3 | 30 |
| Human PKD4 | 31 |
| Human PKD5 | 32 |
| Mouse PKD1 | 37 |
| Mouse PKD2 | 38 |
| Mouse PKD3 | 39 |
| Mouse PKD4 | 40 |
| Mouse PKD5 | 41 |
| Orangutan PKD1 | 43 |
| Orangutan PKD2 | 44 |
| Orangutan PKD3 | 45 |
| Orangutan PKD4 | 46 |
| Orangutan PKD5 | 47 |

In some embodiments, the AAVR, or fragment or derivative thereof, comprises a polycystic kidney disease 1 (PKD1) domain, polycystic kidney disease 2 (PKD2) domain, a polycystic kidney disease 3 (PKD3) domain, a polycystic kidney disease 4 (PKD4) domain, a polycystic kidney disease 5 (PKD5) domain, or a combination thereof.

In some embodiments, the AAVR, or fragment or derivative thereof comprises a PKD1 and a PKD2 domain. In some embodiments, the AAVR, or fragment or derivative thereof comprises a PKD1 and a PKD2 domain having an amino acid sequence of SEQ ID NO: 92. In some embodiments, the AAVR, or fragment or derivative thereof comprises a PKD1 and a PKD3 domain. In some embodiments, the AAVR, or fragment or derivative thereof comprises a PKD1 and a PKD4 domain. In some embodiments, the AAVR, or fragment or derivative thereof comprises a PKD1 and a PKD5 domain. In some embodiments, the AAVR, or fragment or derivative thereof comprises a PKD2 and a PKD3 domain. In some embodiments, the AAVR, or fragment or derivative thereof comprises a PKD2 and a PKD4 domain. In some embodiments, the AAVR, or fragment or derivative thereof comprises a PKD2 and a PKD5 domain. In some embodiments, the AAVR, or fragment or derivative thereof comprises a PKD3 and a PKD4 domain. In some embodiments, the AAVR, or fragment or derivative thereof comprises a PKD3 and a PKD5 domain. In some embodiments, the AAVR, or fragment or derivative thereof comprises a PKD4 and a PKD5 domain. In some embodiments, the AAVR, or fragment or derivative thereof comprises three PKD domains, wherein each PKD domain is independently selected from any one of PKD1-5. In some embodiments, the AAVR, or fragment or derivative thereof comprises four PKD domains, wherein each PKD domain is independently selected from any one of PKD1-5. In some embodiments, the AAVR, or fragment or derivative thereof comprises five PKD domains, wherein each PKD domain is independently selected from any one of PKD1-5. In some embodiments, the AAVR, or fragment or derivative thereof more than five PKD domains, wherein each PKD domain is independently selected from any one of PKD1-5.

In any of the embodiments of the preceding paragraph, each of the PKD domains may be independently selected from a wildtype or a mutant PKD domain. In some embodiments, each PKD may have at least 70%, at least 80%, at least 85%, at least 90%, at least 95%, at least 96%, at least 97%, at least 98%, at least 99%, or at least 100% sequence identity to a wild type PKD. In some embodiments, the AAVR, or fragment or derivative thereof disclosed herein comprise an amino acid sequence with at least about 70%, at least about 75%, at least about 80%, at least about 85%, at least about 90%, or at least about 95% sequence identity to a wild type PKD. In some embodiment, the AAVR, or fragment or derivative thereof bind to AAV using one or more PKDs.

In some embodiments, the AAVR, or fragment or derivative thereof described herein comprise at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25 amino acid mutations relative to a wild-type AAVR or PKD thereof. In some embodiments, the AAVR, or fragment or derivative thereof comprise up to about 25 amino acid mutations, or more, relative to a wildtype AAVR or PKD thereof. For example, the AAV binding polypeptides may comprise about 25-35, about 35-45, about 45-55, about 55-65, or about 65-75 amino acid mutations relative to a wildtype AAVR.

In some embodiments, the AAVR, or fragment or derivative thereof described herein comprise at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25 amino acid mutations relative to a wild type AAVR or PKD thereof, wherein each mutation comprises a change of a native amino acid residue to a histidine.

In some embodiments, the AAVR, or fragment or derivative thereof comprises a sequence of SEQ ID NO: 29. In some embodiments, the AAVR, or fragment or derivative thereof comprises a sequence of SEQ ID NO: 29, wherein at least one amino acid (i.e., a non-histidine amino acid) is mutated to histidine.

In some embodiments, the AAVR, or fragment or derivative thereof comprises SEQ ID NO: 35 with at least one, at least two, at least three, at least four, or at least five mutations. In some embodiments, each mutation is individually selected from the group consisting of V440H, S431H, Q432H, T434H, Y442H, I462H, D435H, D436H, K438H, and I439H.

In some embodiments, the AAVR, or fragment or derivative thereof comprises amino acids 411 to 499 of SEQ ID NO: 35 with at least one, at least two, at least three, at least four, or at least five mutations, wherein each mutation is individually selected from the group consisting of V440H, S431H, Q432H, T434H, Y442H, 1462H, D435H, D436H, K438H, and I439H.

In some embodiments, the AAVR, having an amino acid sequence of SEQ ID NO: 35, or a fragment or derivative thereof, has one or more of the combinations of mutations in Table 4. Each row in Table 4 signifies a combination of mutations.

TABLE 4
Combinations of Mutations

| Mutation 1 | Mutation 2 | Mutation 3 | Mutation 4 | Mutation 5 |
|---|---|---|---|---|
| S431H | V440H | | | |
| Q432H | V440H | | | |
| Q432H | S431H | | | |
| T434H | V440H | | | |
| T434H | S431H | | | |
| T434H | Q432H | | | |
| Y442H | V440H | | | |
| Y442H | S431H | | | |
| Y442H | Q432H | | | |
| Y442H | T434H | | | |
| I462H | V440H | | | |
| I462H | S431H | | | |
| I462H | Q432H | | | |
| I462H | T434H | | | |
| I462H | Y442H | | | |
| D435H | V440H | | | |
| D435H | S431H | | | |
| D435H | Q432H | | | |
| D435H | T434H | | | |
| D435H | Y442H | | | |
| D436H | V440H | | | |
| D436H | S431H | | | |
| D436H | Q432H | | | |
| D436H | T434H | | | |
| D436H | Y442H | | | |
| D435H | I462H | | | |
| D436H | I462H | | | |
| D436H | D435H | | | |
| I439H | V440H | | | |
| I439H | S431H | | | |
| I439H | Q432H | | | |
| I439H | T434H | | | |
| I439H | Y442H | | | |
| K438H | V440H | | | |
| K438H | S431H | | | |
| K438H | Q432H | | | |
| K438H | T434H | | | |
| I439H | I462H | | | |
| K438H | Y442H | | | |
| I439H | D435H | | | |
| I439H | D436H | | | |
| K438H | I462H | | | |
| K438H | D435H | | | |
| K438H | D436H | | | |
| I439H | K438H | | | |
| S431H | V440H | Q432H | | |
| S431H | V440H | T434H | | |
| Q432H | V440H | T434H | | |
| Q432H | S431H | T434H | | |
| S431H | V440H | Y442H | | |
| Q432H | V440H | Y442H | | |
| Q432H | S431H | Y442H | | |
| T434H | V440H | Y442H | | |
| T434H | S431H | Y442H | | |
| T434H | Q432H | Y442H | | |
| S431H | V440H | I462H | | |
| Q432H | V440H | I462H | | |
| Q432H | S431H | I462H | | |
| T434H | V440H | I462H | | |
| T434H | S431H | I462H | | |
| T434H | Q432H | I462H | | |
| Y442H | V440H | I462H | | |
| Y442H | S431H | I462H | | |
| Y442H | Q432H | I462H | | |
| Y442H | T434H | I462H | | |
| S431H | V440H | D435H | | |
| Q432H | V440H | D435H | | |
| Q432H | S431H | D435H | | |
| T434H | V440H | D435H | | |
| T434H | S431H | D435H | | |
| T434H | Q432H | D435H | | |
| Y442H | V440H | D435H | | |
| Y442H | S431H | D435H | | |
| Y442H | Q432H | D435H | | |
| Y442H | T434H | D435H | | |
| S431H | V440H | D436H | | |
| Q432H | V440H | D436H | | |
| Q432H | S431H | D436H | | |
| T434H | V440H | D436H | | |
| T434H | S431H | D436H | | |
| T434H | Q432H | D436H | | |
| Y442H | V440H | D436H | | |
| Y442H | S431H | D436H | | |
| Y442H | Q432H | D436H | | |
| Y442H | T434H | D436H | | |
| I462H | V440H | D435H | | |
| I462H | S431H | D435H | | |
| I462H | Q432H | D435H | | |
| I462H | T434H | D435H | | |
| I462H | Y442H | D435H | | |
| I462H | V440H | D436H | | |
| I462H | S431H | D436H | | |
| I462H | Q432H | D436H | | |
| I462H | T434H | D436H | | |
| I462H | Y442H | D436H | | |
| D435H | V440H | D436H | | |
| D435H | S431H | D436H | | |
| D435H | Q432H | D436H | | |
| D435H | T434H | D436H | | |
| D435H | Y442H | D436H | | |
| D435H | I462H | D436H | | |
| S431H | V440H | I439H | | |
| Q432H | V440H | I439H | | |
| Q432H | S431H | I439H | | |
| T434H | V440H | I439H | | |
| T434H | S431H | I439H | | |
| T434H | Q432H | I439H | | |
| Y442H | V440H | I439H | | |
| Y442H | S431H | I439H | | |
| Y442H | Q432H | I439H | | |
| Y442H | T434H | I439H | | |
| S431H | V440H | K438H | | |
| Q432H | V440H | K438H | | |
| Q432H | S431H | K438H | | |
| T434H | V440H | K438H | | |
| T434H | S431H | K438H | | |
| T434H | Q432H | K438H | | |
| I462H | V440H | I439H | | |
| I462H | S431H | I439H | | |
| Y442H | V440H | K438H | | |
| I462H | Q432H | I439H | | |
| Y442H | S431H | K438H | | |
| I462H | T434H | I439H | | |
| Y442H | Q432H | K438H | | |
| Y442H | T434H | K438H | | |
| I462H | Y442H | I439H | | |
| D435H | V440H | I439H | | |
| D435H | S431H | I439H | | |
| D435H | Q432H | I439H | | |
| D435H | T434H | I439H | | |
| D435H | Y442H | I439H | | |
| D436H | V440H | I439H | | |
| D436H | S431H | I439H | | |
| D436H | Q432H | I439H | | |
| D436H | T434H | I439H | | |
| I462H | V440H | K438H | | |
| I462H | S431H | K438H | | |
| I462H | Q432H | K438H | | |
| I462H | T434H | K438H | | |
| D436H | Y442H | I439H | | |
| I462H | Y442H | K438H | | |
| D435H | V440H | K438H | | |
| D435H | S431H | K438H | | |
| D435H | Q432H | K438H | | |
| D435H | T434H | K438H | | |
| D435H | I462H | I439H | | |
| D435H | Y442H | K438H | | |
| D436H | V440H | K438H | | |
| D436H | S431H | K438H | | |
| D436H | Q432H | K438H | | |
| D436H | T434H | K438H | | |
| D436H | I462H | I439H | | |
| D436H | Y442H | K438H | | |
| D436H | D435H | I439H | | |
| D435H | I462H | K438H | | |
| D436H | I462H | K438H | | |
| D436H | D435H | K438H | | |
| I439H | V440H | K438H | | |
| I439H | S431H | K438H | | |
| I439H | Q432H | K438H | | |
| I439H | T434H | K438H | | |
| I439H | Y442H | K438H | | |
| I439H | I462H | K438H | | |
| I439H | D435H | K438H | | |
| I439H | D436H | K438H | | |
| S431H | V440H | Q432H | T434H | |
| S431H | V440H | Q432H | Y442H | |
| S431H | V440H | T434H | Y442H | |
| Q432H | V440H | T434H | Y442H | |
| Q432H | S431H | T434H | Y442H | |
| S431H | V440H | Q432H | I462H | |
| S431H | V440H | T434H | I462H | |
| Q432H | V440H | T434H | I462H | |
| Q432H | S431H | T434H | I462H | |
| S431H | V440H | Y442H | I462H | |
| Q432H | V440H | Y442H | I462H | |
| Q432H | S431H | Y442H | I462H | |
| T434H | V440H | Y442H | I462H | |
| T434H | S431H | Y442H | I462H | |
| T434H | Q432H | Y442H | I462H | |
| S431H | V440H | Q432H | D435H | |
| S431H | V440H | T434H | D435H | |
| Q432H | V440H | T434H | D435H | |
| Q432H | S431H | T434H | D435H | |
| S431H | V440H | Y442H | D435H | |
| Q432H | V440H | Y442H | D435H | |
| Q432H | S431H | Y442H | D435H | |
| T434H | V440H | Y442H | D435H | |
| T434H | S431H | Y442H | D435H | |
| T434H | Q432H | Y442H | D435H | |
| S431H | V440H | Q432H | D436H | |
| S431H | V440H | T434H | D436H | |
| Q432H | V440H | T434H | D436H | |
| Q432H | S431H | T434H | D436H | |
| S431H | V440H | Y442H | D436H | |
| Q432H | V440H | Y442H | D436H | |
| Q432H | S431H | Y442H | D436H | |
| T434H | V440H | Y442H | D436H | |
| T434H | S431H | Y442H | D436H | |
| T434H | Q432H | Y442H | D436H | |
| S431H | V440H | I462H | D435H | |
| Q432H | V440H | I462H | D435H | |
| Q432H | S431H | I462H | D435H | |
| T434H | V440H | I462H | D435H | |
| T434H | S431H | I462H | D435H | |
| T434H | Q432H | I462H | D435H | |
| Y442H | V440H | I462H | D435H | |
| Y442H | S431H | I462H | D435H | |
| Y442H | Q432H | I462H | D435H | |
| Y442H | T434H | I462H | D435H | |
| S431H | V440H | I462H | D436H | |
| Q432H | V440H | I462H | D436H | |
| Q432H | S431H | I462H | D436H | |
| T434H | V440H | I462H | D436H | |
| T434H | S431H | I462H | D436H | |
| T434H | Q432H | I462H | D436H | |
| Y442H | V440H | I462H | D436H | |
| Y442H | S431H | I462H | D436H | |
| Y442H | Q432H | I462H | D436H | |
| Y442H | T434H | I462H | D436H | |
| S431H | V440H | D435H | D436H | |
| Q432H | V440H | D435H | D436H | |
| Q432H | S431H | D435H | D436H | |
| T434H | V440H | D435H | D436H | |
| T434H | S431H | D435H | D436H | |
| T434H | Q432H | D435H | D436H | |
| Y442H | V440H | D435H | D436H | |
| Y442H | S431H | D435H | D436H | |
| Y442H | Q432H | D435H | D436H | |
| Y442H | T434H | D435H | D436H | |
| I462H | V440H | D435H | D436H | |
| I462H | S431H | D435H | D436H | |
| I462H | Q432H | D435H | D436H | |
| I462H | T434H | D435H | D436H | |
| I462H | Y442H | D435H | D436H | |
| S431H | V440H | Q432H | I439H | |
| S431H | V440H | T434H | I439H | |
| Q432H | V440H | T434H | I439H | |
| Q432H | S431H | T434H | I439H | |
| S431H | V440H | Y442H | I439H | |
| Q432H | V440H | Y442H | I439H | |
| Q432H | S431H | Y442H | I439H | |
| T434H | V440H | Y442H | I439H | |
| T434H | S431H | Y442H | I439H | |
| T434H | Q432H | Y442H | I439H | |
| S431H | V440H | Q432H | K438H | |
| S431H | V440H | T434H | K438H | |
| Q432H | V440H | T434H | K438H | |
| Q432H | S431H | T434H | K438H | |
| S431H | V440H | I462H | I439H | |
| Q432H | V440H | I462H | I439H | |
| S431H | V440H | Y442H | K438H | |
| Q432H | S431H | I462H | I439H | |
| T434H | V440H | I462H | I439H | |
| Q432H | V440H | Y442H | K438H | |
| T434H | S431H | I462H | I439H | |
| Q432H | S431H | Y442H | K438H | |
| T434H | V440H | Y442H | K438H | |
| T434H | Q432H | I462H | I439H | |
| T434H | S431H | Y442H | K438H | |
| T434H | Q432H | Y442H | K438H | |
| Y442H | V440H | I462H | I439H | |
| Y442H | S431H | I462H | I439H | |
| Y442H | Q432H | I462H | I439H | |
| Y442H | T434H | I462H | I439H | |
| S431H | V440H | D435H | I439H | |
| Q432H | V440H | D435H | I439H | |
| Q432H | S431H | D435H | I439H | |
| T434H | V440H | D435H | I439H | |
| T434H | S431H | D435H | I439H | |
| T434H | Q432H | D435H | I439H | |
| Y442H | V440H | D435H | I439H | |
| Y442H | S431H | D435H | I439H | |
| Y442H | Q432H | D435H | I439H | |
| Y442H | T434H | D435H | I439H | |
| S431H | V440H | D436H | I439H | |
| Q432H | V440H | D436H | I439H | |
| Q432H | S431H | D436H | I439H | |
| T434H | V440H | D436H | I439H | |
| T434H | S431H | D436H | I439H | |
| T434H | Q432H | D436H | I439H | |
| S431H | V440H | I462H | K438H | |
| Q432H | V440H | I462H | K438H | |
| Q432H | S431H | I462H | K438H | |
| T434H | V440H | I462H | K438H | |
| T434H | S431H | I462H | K438H | |
| T434H | Q432H | I462H | K438H | |
| Y442H | V440H | D436H | I439H | |
| Y442H | S431H | D436H | I439H | |
| Y442H | Q432H | D436H | I439H | |
| Y442H | T434H | D436H | I439H | |
| Y442H | V440H | I462H | K438H | |
| Y442H | S431H | I462H | K438H | |
| Y442H | Q432H | I462H | K438H | |
| Y442H | T434H | I462H | K438H | |
| S431H | V440H | D435H | K438H | |
| Q432H | V440H | D435H | K438H | |
| Q432H | S431H | D435H | K438H | |
| T434H | V440H | D435H | K438H | |
| T434H | S431H | D435H | K438H | |
| T434H | Q432H | D435H | K438H | |
| I462H | V440H | D435H | I439H | |
| I462H | S431H | D435H | I439H | |
| Y442H | V440H | D435H | K438H | |
| I462H | Q432H | D435H | I439H | |
| Y442H | S431H | D435H | K438H | |
| I462H | T434H | D435H | I439H | |
| Y442H | Q432H | D435H | K438H | |
| Y442H | T434H | D435H | K438H | |
| I462H | Y442H | D435H | I439H | |
| S431H | V440H | D436H | K438H | |
| Q432H | V440H | D436H | K438H | |
| Q432H | S431H | D436H | K438H | |
| T434H | V440H | D436H | K438H | |
| T434H | S431H | D436H | K438H | |
| T434H | Q432H | D436H | K438H | |
| I462H | V440H | D436H | I439H | |
| I462H | S431H | D436H | I439H | |
| Y442H | V440H | D436H | K438H | |
| I462H | Q432H | D436H | I439H | |
| Y442H | S431H | D436H | K438H | |
| I462H | T434H | D436H | I439H | |
| Y442H | Q432H | D436H | K438H | |
| Y442H | T434H | D436H | K438H | |
| I462H | Y442H | D436H | I439H | |
| D435H | V440H | D436H | I439H | |
| D435H | S431H | D436H | I439H | |
| D435H | Q432H | D436H | I439H | |
| D435H | T434H | D436H | I439H | |
| I462H | V440H | D435H | K438H | |
| I462H | S431H | D435H | K438H | |
| I462H | Q432H | D435H | K438H | |
| I462H | T434H | D435H | K438H | |
| D435H | Y442H | D436H | I439H | |
| I462H | Y442H | D435H | K438H | |
| I462H | V440H | D436H | K438H | |
| I462H | S431H | D436H | K438H | |
| I462H | Q432H | D436H | K438H | |
| I462H | T434H | D436H | K438H | |
| I462H | Y442H | D436H | K438H | |
| D435H | V440H | D436H | K438H | |
| D435H | S431H | D436H | K438H | |
| D435H | Q432H | D436H | K438H | |
| D435H | T434H | D436H | K438H | |
| D435H | I462H | D436H | I439H | |
| D435H | Y442H | D436H | K438H | |
| D435H | I462H | D436H | K438H | |
| S431H | V440H | I439H | K438H | |
| Q432H | V440H | I439H | K438H | |
| Q432H | S431H | I439H | K438H | |
| T434H | V440H | I439H | K438H | |
| T434H | S431H | I439H | K438H | |
| T434H | Q432H | I439H | K438H | |
| Y442H | V440H | I439H | K438H | |
| Y442H | S431H | I439H | K438H | |
| Y442H | Q432H | I439H | K438H | |
| Y442H | T434H | I439H | K438H | |
| I462H | V440H | I439H | K438H | |
| I462H | S431H | I439H | K438H | |
| I462H | Q432H | I439H | K438H | |
| I462H | T434H | I439H | K438H | |
| I462H | Y442H | I439H | K438H | |
| D435H | V440H | I439H | K438H | |
| D435H | S431H | I439H | K438H | |
| D435H | Q432H | I439H | K438H | |
| D435H | T434H | I439H | K438H | |
| D435H | Y442H | I439H | K438H | |
| D436H | V440H | I439H | K438H | |
| D436H | S431H | I439H | K438H | |
| D436H | Q432H | I439H | K438H | |
| D436H | T434H | I439H | K438H | |
| D436H | Y442H | I439H | K438H | |
| D435H | I462H | I439H | K438H | |
| D436H | I462H | I439H | K438H | |
| D436H | D435H | I439H | K438H | |
| S431H | V440H | Q432H | T434H | Y442H |
| S431H | V440H | Q432H | T434H | I462H |
| S431H | V440H | Q432H | Y442H | I462H |
| S431H | V440H | T434H | Y442H | I462H |
| Q432H | V440H | T434H | Y442H | I462H |
| Q432H | S431H | T434H | Y442H | I462H |
| S431H | V440H | Q432H | T434H | D435H |
| S431H | V440H | Q432H | Y442H | D435H |
| S431H | V440H | T434H | Y442H | D435H |
| Q432H | V440H | T434H | Y442H | D435H |
| Q432H | S431H | T434H | Y442H | D435H |
| S431H | V440H | Q432H | T434H | D436H |
| S431H | V440H | Q432H | Y442H | D436H |
| S431H | V440H | T434H | Y442H | D436H |
| Q432H | V440H | T434H | Y442H | D436H |
| Q432H | S431H | T434H | Y442H | D436H |
| S431H | V440H | Q432H | I462H | D435H |
| S431H | V440H | T434H | I462H | D435H |
| Q432H | V440H | T434H | I462H | D435H |
| Q432H | S431H | T434H | I462H | D435H |
| S431H | V440H | Y442H | I462H | D435H |
| Q432H | V440H | Y442H | I462H | D435H |
| Q432H | S431H | Y442H | I462H | D435H |
| T434H | V440H | Y442H | I462H | D435H |
| T434H | S431H | Y442H | I462H | D435H |
| T434H | Q432H | Y442H | I462H | D435H |
| S431H | V440H | Q432H | I462H | D436H |
| S431H | V440H | T434H | I462H | D436H |
| Q432H | V440H | T434H | I462H | D436H |
| Q432H | S431H | T434H | I462H | D436H |
| S431H | V440H | Y442H | I462H | D436H |
| Q432H | V440H | Y442H | I462H | D436H |
| Q432H | S431H | Y442H | I462H | D436H |
| T434H | V440H | Y442H | I462H | D436H |
| T434H | S431H | Y442H | I462H | D436H |
| T434H | Q432H | Y442H | I462H | D436H |
| S431H | V440H | Q432H | D435H | D436H |
| S431H | V440H | T434H | D435H | D436H |
| Q432H | V440H | T434H | D435H | D436H |
| Q432H | S431H | T434H | D435H | D436H |
| S431H | V440H | Y442H | D435H | D436H |
| Q432H | V440H | Y442H | D435H | D436H |
| Q432H | S431H | Y442H | D435H | D436H |
| T434H | V440H | Y442H | D435H | D436H |
| T434H | S431H | Y442H | D435H | D436H |
| T434H | Q432H | Y442H | D435H | D436H |
| S431H | V440H | I462H | D435H | D436H |
| Q432H | V440H | I462H | D435H | D436H |
| Q432H | S431H | I462H | D435H | D436H |
| T434H | V440H | I462H | D435H | D436H |
| T434H | S431H | I462H | D435H | D436H |
| T434H | Q432H | I462H | D435H | D436H |
| Y442H | V440H | I462H | D435H | D436H |
| Y442H | S431H | I462H | D435H | D436H |
| Y442H | Q432H | I462H | D435H | D436H |
| Y442H | T434H | I462H | D435H | D436H |
| S431H | V440H | Q432H | T434H | I439H |
| S431H | V440H | Q432H | Y442H | I439H |
| S431H | V440H | T434H | Y442H | I439H |
| Q432H | V440H | T434H | Y442H | I439H |
| Q432H | S431H | T434H | Y442H | I439H |
| S431H | V440H | Q432H | T434H | K438H |
| S431H | V440H | Q432H | I462H | I439H |
| S431H | V440H | T434H | I462H | I439H |
| S431H | V440H | Q432H | Y442H | K438H |
| Q432H | V440H | T434H | I462H | I439H |
| S431H | V440H | T434H | Y442H | K438H |
| Q432H | S431H | T434H | I462H | I439H |
| Q432H | V440H | T434H | Y442H | K438H |
| Q432H | S431H | T434H | Y442H | K438H |
| S431H | V440H | Y442H | I462H | I439H |
| Q432H | V440H | Y442H | I462H | I439H |
| Q432H | S431H | Y442H | I462H | I439H |
| T434H | V440H | Y442H | I462H | I439H |
| T434H | S431H | Y442H | I462H | I439H |
| T434H | Q432H | Y442H | I462H | I439H |
| S431H | V440H | Q432H | D435H | I439H |
| S431H | V440H | T434H | D435H | I439H |
| Q432H | V440H | T434H | D435H | I439H |
| Q432H | S431H | T434H | D435H | I439H |
| S431H | V440H | Y442H | D435H | I439H |
| Q432H | V440H | Y442H | D435H | I439H |
| Q432H | S431H | Y442H | D435H | I439H |
| T434H | V440H | Y442H | D435H | I439H |
| T434H | S431H | Y442H | D435H | I439H |
| T434H | Q432H | Y442H | D435H | I439H |
| S431H | V440H | Q432H | D436H | I439H |
| S431H | V440H | T434H | D436H | I439H |
| Q432H | V440H | T434H | D436H | I439H |
| Q432H | S431H | T434H | D436H | I439H |
| S431H | V440H | Q432H | I462H | K438H |
| S431H | V440H | T434H | I462H | K438H |
| Q432H | V440H | T434H | I462H | K438H |
| Q432H | S431H | T434H | I462H | K438H |
| S431H | V440H | Y442H | D436H | I439H |
| Q432H | V440H | Y442H | D436H | I439H |
| Q432H | S431H | Y442H | D436H | I439H |
| T434H | V440H | Y442H | D436H | I439H |
| T434H | S431H | Y442H | D436H | I439H |
| T434H | Q432H | Y442H | D436H | I439H |
| S431H | V440H | Y442H | I462H | K438H |
| Q432H | V440H | Y442H | I462H | K438H |
| Q432H | S431H | Y442H | I462H | K438H |
| T434H | V440H | Y442H | I462H | K438H |
| T434H | S431H | Y442H | I462H | K438H |
| T434H | Q432H | Y442H | I462H | K438H |
| S431H | V440H | Q432H | D435H | K438H |
| S431H | V440H | T434H | D435H | K438H |
| Q432H | V440H | T434H | D435H | K438H |
| Q432H | S431H | T434H | D435H | K438H |
| S431H | V440H | I462H | D435H | I439H |
| Q432H | V440H | I462H | D435H | I439H |
| S431H | V440H | Y442H | D435H | K438H |
| Q432H | S431H | I462H | D435H | I439H |
| T434H | V440H | I462H | D435H | I439H |
| Q432H | V440H | Y442H | D435H | K438H |
| T434H | S431H | I462H | D435H | I439H |
| Q432H | S431H | Y442H | D435H | K438H |
| T434H | V440H | Y442H | D435H | K438H |
| T434H | Q432H | I462H | D435H | I439H |
| T434H | S431H | Y442H | D435H | K438H |
| T434H | Q432H | Y442H | D435H | K438H |
| Y442H | V440H | I462H | D435H | I439H |
| Y442H | S431H | I462H | D435H | I439H |
| Y442H | Q432H | I462H | D435H | I439H |
| Y442H | T434H | I462H | D435H | I439H |
| S431H | V440H | Q432H | D436H | K438H |
| S431H | V440H | T434H | D436H | K438H |
| Q432H | V440H | T434H | D436H | K438H |
| Q432H | S431H | T434H | D436H | K438H |
| S431H | V440H | I462H | D436H | I439H |
| Q432H | V440H | I462H | D436H | I439H |
| S431H | V440H | Y442H | D436H | K438H |
| Q432H | S431H | I462H | D436H | I439H |
| T434H | V440H | I462H | D436H | I439H |
| Q432H | V440H | Y442H | D436H | K438H |
| T434H | S431H | I462H | D436H | I439H |
| Q432H | S431H | Y442H | D436H | K438H |
| T434H | V440H | Y442H | D436H | K438H |
| T434H | Q432H | I462H | D436H | I439H |
| T434H | S431H | Y442H | D436H | K438H |
| T434H | Q432H | Y442H | D436H | K438H |
| Y442H | V440H | I462H | D436H | I439H |
| Y442H | S431H | I462H | D436H | I439H |
| Y442H | Q432H | I462H | D436H | I439H |
| Y442H | T434H | I462H | D436H | I439H |
| S431H | V440H | D435H | D436H | I439H |
| Q432H | V440H | D435H | D436H | I439H |
| Q432H | S431H | D435H | D436H | I439H |
| T434H | V440H | D435H | D436H | I439H |
| T434H | S431H | D435H | D436H | I439H |
| T434H | Q432H | D435H | D436H | I439H |
| S431H | V440H | I462H | D435H | K438H |
| Q432H | V440H | I462H | D435H | K438H |
| Q432H | S431H | I462H | D435H | K438H |
| T434H | V440H | I462H | D435H | K438H |
| T434H | S431H | I462H | D435H | K438H |
| T434H | Q432H | I462H | D435H | K438H |
| Y442H | V440H | D435H | D436H | I439H |
| Y442H | S431H | D435H | D436H | I439H |
| Y442H | Q432H | D435H | D436H | I439H |
| Y442H | T434H | D435H | D436H | I439H |
| Y442H | V440H | I462H | D435H | K438H |
| Y442H | S431H | I462H | D435H | K438H |
| Y442H | Q432H | I462H | D435H | K438H |
| Y442H | T434H | I462H | D435H | K438H |
| S431H | V440H | I462H | D436H | K438H |
| Q432H | V440H | I462H | D436H | K438H |
| Q432H | S431H | I462H | D436H | K438H |
| T434H | V440H | I462H | D436H | K438H |
| T434H | S431H | I462H | D436H | K438H |
| T434H | Q432H | I462H | D436H | K438H |
| Y442H | V440H | I462H | D436H | K438H |
| Y442H | S431H | I462H | D436H | K438H |
| Y442H | Q432H | I462H | D436H | K438H |
| Y442H | T434H | I462H | D436H | K438H |
| S431H | V440H | D435H | D436H | K438H |
| Q432H | V440H | D435H | D436H | K438H |
| Q432H | S431H | D435H | D436H | K438H |
| T434H | V440H | D435H | D436H | K438H |
| T434H | S431H | D435H | D436H | K438H |
| T434H | Q432H | D435H | D436H | K438H |
| I462H | V440H | D435H | D436H | I439H |
| I462H | S431H | D435H | D436H | I439H |
| Y442H | V440H | D435H | D436H | K438H |
| I462H | Q432H | D435H | D436H | I439H |
| Y442H | S431H | D435H | D436H | K438H |
| I462H | T434H | D435H | D436H | I439H |
| Y442H | Q432H | D435H | D436H | K438H |
| Y442H | T434H | D435H | D436H | K438H |
| I462H | Y442H | D435H | D436H | I439H |
| I462H | V440H | D435H | D436H | K438H |
| I462H | S431H | D435H | D436H | K438H |
| I462H | Q432H | D435H | D436H | K438H |
| I462H | T434H | D435H | D436H | K438H |
| I462H | Y442H | D435H | D436H | K438H |
| S431H | V440H | Q432H | I439H | K438H |
| S431H | V440H | T434H | I439H | K438H |
| Q432H | V440H | T434H | I439H | K438H |
| Q432H | S431H | T434H | I439H | K438H |
| S431H | V440H | Y442H | I439H | K438H |
| Q432H | V440H | Y442H | I439H | K438H |
| Q432H | S431H | Y442H | I439H | K438H |
| T434H | V440H | Y442H | I439H | K438H |
| T434H | S431H | Y442H | I439H | K438H |
| T434H | Q432H | Y442H | I439H | K438H |
| S431H | V440H | I462H | I439H | K438H |
| Q432H | V440H | I462H | I439H | K438H |
| Q432H | S431H | I462H | I439H | K438H |
| T434H | V440H | I462H | I439H | K438H |
| T434H | S431H | I462H | I439H | K438H |
| T434H | Q432H | I462H | I439H | K438H |
| Y442H | V440H | I462H | I439H | K438H |
| Y442H | S431H | I462H | I439H | K438H |
| Y442H | Q432H | I462H | I439H | K438H |
| Y442H | T434H | I462H | I439H | K438H |
| S431H | V440H | D435H | I439H | K438H |
| Q432H | V440H | D435H | I439H | K438H |
| Q432H | S431H | D435H | I439H | K438H |
| T434H | V440H | D435H | I439H | K438H |
| T434H | S431H | D435H | I439H | K438H |
| T434H | Q432H | D435H | I439H | K438H |
| Y442H | V440H | D435H | I439H | K438H |
| Y442H | S431H | D435H | I439H | K438H |
| Y442H | Q432H | D435H | I439H | K438H |
| Y442H | T434H | D435H | I439H | K438H |
| S431H | V440H | D436H | I439H | K438H |
| Q432H | V440H | D436H | I439H | K438H |
| Q432H | S431H | D436H | I439H | K438H |
| T434H | V440H | D436H | I439H | K438H |
| T434H | S431H | D436H | I439H | K438H |
| T434H | Q432H | D436H | I439H | K438H |
| Y442H | V440H | D436H | I439H | K438H |
| Y442H | S431H | D436H | I439H | K438H |
| Y442H | Q432H | D436H | I439H | K438H |
| Y442H | T434H | D436H | I439H | K438H |
| I462H | V440H | D435H | I439H | K438H |
| I462H | S431H | D435H | I439H | K438H |
| T462H | Q432H | D435H | I439H | K438H |
| I462H | T434H | D435H | I439H | K438H |
| I462H | Y442H | D435H | I439H | K438H |
| I462H | V440H | D436H | I439H | K438H |
| I462H | S431H | D436H | I439H | K438H |
| I462H | Q432H | D436H | I439H | K438H |
| I462H | T434H | D436H | I439H | K438H |
| I462H | Y442H | D436H | I439H | K438H |
| D435H | V440H | D436H | I439H | K438H |
| D435H | S431H | D436H | I439H | K438H |
| D435H | Q432H | D436H | I439H | K438H |
| D435H | T434H | D436H | I439H | K438H |
| D435H | Y442H | D436H | I439H | K438H |
| D435H | I462H | D436H | I439H | K438H |

In some embodiments, the AAVR, or fragment or derivative thereof, comprises SEQ ID NO: 29 with at least one, at least two, at least three, at least four, or at least five mutations, wherein each mutation is individually selected from the group consisting of S23H, Q24H, T26H, D29H, K30H, I31H, V32H, Y34H, and 154H. In some embodiments, the AAVR, or fragment or derivative thereof, comprises SEQ ID NO: 29 with V32H and V34H mutations. In some embodiments, the AAVR, or fragment or derivative thereof, comprises SEQ ID NO: 29 with S23H and Q24H mutations. In some embodiments, the AAVR, or fragment or derivative thereof, comprises SEQ ID NO: 29 with at least one, at least two, at least three, at least four, or at least five mutations, wherein each mutation is individually selected from the group consisting of S23H, Q24H, T26H, D29H, K30H, I31H, V32H, Y34H, and I54H. In some embodiments, the AAVR, or fragment or derivative thereof, comprises an amino acid sequence of any one of SEQ ID NO: 76-86.

In some embodiments, the AAVR, or fragment or derivative thereof, comprises SEQ ID NO: 29 with an additional five amino acids at the N-terminus having an amino acid sequence of GNRPP (SEQ ID NO: 89), and an additional five amino acids at the C-terminus having an amino acid sequence of VDYPG (SEQ ID NO: 90). In some embodiments, the AAVR, or fragment or derivative thereof, comprises an amino acid sequence of SEQ ID NO: 52. In some embodiments, the AAVR, or fragment or derivative thereof, comprises SEQ ID NO: 29 with at least one, at least two, at least three, at least four, or at least five mutations, wherein each mutation is individually selected from the group consisting of S23H, Q24H, T26H, D29H, K30H, I31H, V32H, Y34H, and 154H and an additional five amino acids at the N-terminus having an amino acid sequence of GNRPP (SEQ ID NO: 89), and an additional five amino acids at the C-terminus having an amino acid sequence of VDYPG (SEQ ID NO: 90). In some embodiments, the AAVR, or fragment or derivative thereof, comprises SEQ ID NO: 52 with at least one, at least two, at least three, at least four, or at least five mutations, wherein each mutation is individually selected from the group consisting of S23H, Q24H, T26H, D29H, K30H, I31H, V32H, Y34H, and I54H.

In some embodiments, the AAVR, or fragment or derivative thereof, is encoded by a nucleic acid sequence of any one of SEQ ID NO: 93-116. In some embodiments, the AAVR, or fragment or derivative thereof, comprises SEQ ID NO: 29 with V32H and V34H mutations and an additional five amino acids at the N-terminus having an amino acid sequence of GNRPP (SEQ ID NO: 89), and an additional five amino acids at the C-terminus having an amino acid sequence of VDYPG (SEQ ID NO: 90). In some embodiments, the AAVR, or fragment or derivative thereof, comprises SEQ ID NO: 29 with S23H and Q24H mutations and an additional five amino acids at the N-terminus having an amino acid sequence of GNRPP (SEQ ID NO: 89), and an additional five amino acids at the C-terminus having an amino acid sequence of VDYPG (SEQ ID NO: 90). In some embodiments, the AAVR, or fragment or derivative thereof, comprising SEQ ID NO: 29 with at least one, at least two, at least three, at least four, or at least five mutations, wherein each mutation is individually selected from the group consisting of S23H, Q24H, T26H, D29H, K30H, I31H, V32H, Y34H, and 154H and an additional five amino acids at the N-terminus having an amino acid sequence of GNRPP (SEQ ID NO: 89), and an additional five amino acids at the C-terminus having an amino acid sequence of VDYPG (SEQ ID NO: 90). In some embodiments, the AAVR, or fragment or derivative thereof, comprises an amino acid sequence of any one of SEQ ID NOs: 53-63.

In some embodiments, the AAVR, or fragment or derivative thereof, comprises an amino acid sequence of SEQ ID NO: 29, plus an additional five amino acids at the N-terminus having an amino acid sequence of GNRPP (SEQ ID NO: 89), and an additional four amino acids at the C-terminus having an amino acid sequence of VDYP (SEQ ID NO: 91). In some embodiments, the AAVR, or fragment or derivative thereof, comprises an amino acid sequence of SEQ ID NO: 64. In some embodiments, the AAVR, or fragment or derivative thereof, comprises an amino acid sequence of SEQ ID NO: 29 with at least one, at least two, at least three, at least four, or at least five mutations, wherein each mutation is individually selected from the group consisting of S23H, Q24H, T26H, D29H, K30H, I31H, V32H, Y34H, and 154H, and an additional five amino acids at the N-terminus having an amino acid sequence of GNRPP (SEQ ID NO: 89), and an additional four amino acids at the C-terminus having an amino acid sequence of VDYP (SEQ ID NO: 91). In some embodiments, the AAVR, or fragment or derivative thereof, comprises SEQ ID NO: 29 plus V32H and V34H mutations and an additional five amino acids at the N-terminus having an amino acid sequence of GNRPP (SEQ ID NO: 89), and an additional four amino acids at the C-terminus having an amino acid sequence of VDYP (SEQ ID NO: 91). In some embodiments, the AAVR, or fragment or derivative thereof, comprises SEQ ID NO: 29 with S23H and Q24H mutations and an additional five amino acids at the N-terminus having an amino acid sequence of GNRPP (SEQ ID NO: 89), and an additional four amino acids at the C-terminus having an amino acid sequence of VDYP (SEQ ID NO: 91). In some embodiments, the AAVR, or fragment or derivative thereof, comprises an amino acid sequence of SEQ ID NO: 29 with at least one, at least two, at least three, at least four, or at least five mutations, wherein each mutation is individually selected from the group consisting of S23H, Q24H, T26H, D29H, K30H, I31H, V32H, Y34H, and I54H and an additional five amino acids at the N-terminus having an amino acid sequence of GNRPP (SEQ ID NO: 89), and an additional four amino acids at the C-terminus having an amino acid sequence of VDYP (SEQ ID NO: 91). In some embodiments, the AAVR, or fragment or derivative thereof, comprises an amino acid sequence of any one of SEQ ID NOS: 65-75.

In some embodiments, the AAVR, or fragment or derivative thereof, described herein comprise one or more MANEC motifs (See, e.g., SEQ ID NOS: 49-51). In some embodiments, the AAVR, or fragment or derivative thereof, described herein comprise one or more recombinant MANEC motifs. In some embodiments, the AAVR, or fragment or derivative thereof, described herein comprise an amino acid sequence with at least about 80%, at least about 90%, at least about 95, or at least about 95% identity to a wild type MANEC motif. In some embodiments, the AAVR, or fragment or derivative thereof, comprise a MANEC motif having at least about 80%, at least about 90% or at least about 95% identity to any one of SEQ ID NOs: 49-51. In some embodiments, the AAVR, or fragment or derivative thereof, binds to AAV particles via one or more MANEC motifs.

In some embodiments, the AAVR, or fragment or derivative thereof, described herein comprises an N-terminal methionine. In some embodiments, the N-terminal methionine initiates translation of the AAVR, or fragment or derivative thereof, described herein. In some embodiments, the AAVR, or fragment or derivative thereof, described herein lacks an N-terminal methionine.

In some embodiments, the AAVR, or fragment or derivative thereof, described herein binds to AAV particles of the following serotypes: AAV1, AAV2, AAV3, AAV4, AAV5, AAV6, AAV7, AAV8, AAV9, AAV10, AAV11, AAV12, AAVrh8, AAVrh10, and/or AAVrh74. In some embodiments, the AAVR, or fragment or derivative thereof, binds to AAV particles of one or more of the following serotypes: AAV1, AAV2, AAV3B, AAV5, AAV6, AAV8, and AAV9. In some embodiments, the AAVR, or fragment or derivative thereof, binds to AAV1 particles. In some embodiments, the AAVR, or fragment or derivative thereof, binds to AAV2 particles. In some embodiments, the AAVR, or fragment or derivative thereof, binds to AAV3B particles. In some embodiments, the AAVR, or fragment or derivative thereof, binds to AAV5 particles. In some embodiments, the AAVR, or fragment or derivative thereof, binds to AAV6 particles. In some embodiments, the AAVR, or fragment or derivative thereof, binds to AAV8 particles. In some embodiments, the AAVR, or fragment or derivative thereof, binds to AAV9 particles.

In some embodiments, the AAVR is the human AAVR. In some embodiments, the AAVR is a primate AAVR. In some embodiments, the AAVR is a wildtype AAVR. In some embodiments, the AAVR is a mutant AAVR.

In some embodiments, the AAVR is a glycoprotein. In some embodiments, the AAVR, or fragment or derivative thereof, described herein comprises one or more glycosylation sites. In some embodiments, the AAVR, or fragment or derivative thereof, comprises O-linked glycosylation sites. In some embodiments, the AAVR, or fragment or derivative thereof, comprises N-linked glycosylation sites. In some embodiments, the AAVR, or fragment or derivative thereof, is glycosylated at one or more asparagine and/or glutamine residues.

In some embodiments, a fusion protein comprises from about 1 to about 100 AAVR, or fragment or derivative thereof. In some embodiments, the number of AAVR, or fragment or derivative thereof within a fusion protein is about 1, about 5, about 10, about 20, about 30, about 40, about 50, about 55, about 60, about 65, about 70, about 75, about 80, about 85, about 90, about 95, or about 100. In some embodiments, a single second polypeptide with phase behavior may be coupled to multiple AAVR, or fragment or derivative thereof, such as about 1 to about 100 AAVR, or fragment or derivative thereof.

In some embodiments, the AAVR, or fragment or derivative thereof, comprises a sequence of any one of SEQ ID NOS: 28-34, 37-41, 43-47, and 52-86. In some embodiments wherein a single polypeptide with phase behavior is coupled to multiple AAVR, or fragment or derivative thereof, each AAVR, or fragment or derivative thereof may be independently selected from SEQ ID NOS: 28-34, 37-41, 43-47, and 52-86.

In some embodiments, the first polypeptide is a polypeptide isolated or derived from SARS-COV-2. In some embodiments, the polypeptide isolated or derived from SARS-COV-2 is the spike, membrane, envelope, or nucleocapsid protein. In some embodiments, the first polypeptide is the SARS-COV-2 spike protein, or a fragment or derivative thereof. The SARS-CoV-2 spike protein (see, e.g. Uniprot Accession No. P0DTC2) mediates fusion of the virion to the cell membrane of a host via human angiotensin converting enzyme 2 (ACE2). An exemplary sequence of a SARS-COV-2 S protein is: MFVFLVLLPLVSSQCVNLTTRTQLPPAYTNSFTRGVYYPDKVFRSSVLHSTQDLFLPFFS NVTWFHAIHVSGTNGTKRFDNPVLPFNDGVYFASTEKSNIIRGWIFGTTLDSKTQSLLIV NNATNVVIKVCEFQFCNDPFLGVYYHKNNKSWMESEFRVYSSANNCTFEYVSQPFLMD LEGKQGNFKNLREFVFKNIDGYFKIYSKHTPINL VRDLPQGFSALEPLVDLPIGINITRFQT LLALHRSYLTPGDSSSGWTAGAAAYYVGYLQPRTFLLKYNENGTITDAVDCALDPLSET KCTLKSFTVEKGIYQTSNFR VQPTESIVRFPNITNLCPFGEVFNATRFASVYAWNRKRISN CVADYSVLYNSASFSTFKCYGVSPTKLNDLCFTNVYADSFVIRGDEVRQIAPGQTGKIA DYNYKLPDDFTGCVIAWNSNNLDSKVGGNYNYLYRLFRKSNLKPFERDISTEIYQAGST PCNGVEGENCYFPLQSYGFQPTNGVGYQPYRVVVLSFELLHAPATVCGPKKSTNLVKN KCVNFNFNGLTGTGVLTESNKKFLPFQQFGRDIADTTDAVRDPQTLEILDITPCSFGGVS VITPGTNTSNQVAVLYQDVNCTEVPVAIHADQLTPTWRVYSTGSNVFQTRAGCLIGAEH VNNSYECDIPIGAGICASYQTQTNSPRRARSVASQSIIAYTMSLGAENSVAYSNNSIAIPT NFTISVTTEILPVSMTKTSVDCTMYICGDSTECSNLLLQYGSFCTQLNRALTGIA VEQDK NTQEVFAQVKQIYKTPPIKDFGGFNFSQILPDPSKPSKRSFIEDLLFNKVTLADAGFIKQY GDCLGDIAARDLICAQKFNGLTVLPPLLTDEMIAQYTSALLAGTITSGWTFGAGAALQIP FAMQMAYRFNGIGVTQNVLYENQKLIANQFNSAIGKIQDSLSSTASALGKLQDVVNQN AQALNTLVKQLSSNFGAISSVLNDILSRLDK VEAEVQIDRLITGRLQSLQTYVTQQLIRAA EIRASANLAATKMSECVLGQSKRVDFCGKGYHLMSFPQSAPHGVVFLHVTYVPAQEKN FTTAPAICHDGKAHFPREGVFVSNGTHWFVTQRNFYEPQIITTDNTFVSGNCDVVIGIVN NTVYDPLQPELDSFKEELDKYFKNHTSPDVDLGDISGINASVVNIQKEIDRLNEVAKNLN ESLIDLQELGKYEQYIKWPWYIWLGFIAGLIAIVMVTIMLCCMTSCCSCLKGCCSCGSCC KFDEDDSEPVLKGVKLHYT (SEQ ID NO: 27). In some embodiments, the first polypeptide is a SARS-COV-2 S protein having an amino acid sequence of SEQ ID NO: 27 or about 95%, about 96%, about 97%, about 98%, or about 99% identity to SEQ ID NO: 27.

### Second Polypeptide

In some embodiments, the disclosure provides a fusion protein comprising a polypeptide that has phase behavior.

In some embodiments, the polypeptide with phase behavior is a resilin-like polypeptide (RLP). Resilin-like polypeptides are elastomeric polypeptides with mechanical properties including desirable resilience, compressive elastic modulus, tensile elastic modulus, shear modulus, extension to break, maximum tensile strength, hardness, rebound, and compression set. In some embodiments, the resilin-like polypeptides described herein are polymers which comprise one or more repeats. In some embodiments, the polymeric repeats may have an amino acid sequence selected from any one of SEQ ID NOS: 1-9.

In some embodiments, a resilin-like polypeptide comprises more than one type of repeat, e.g. a repeat of SEQ ID NO: 1 and a repeat of SEQ ID NO: 3.

In some embodiments, the resilin-like polypeptides described herein comprise repeats that occur up to 500 times within a given RLP. In some embodiments, the repeats occur about 1, about 2, about 3, about 4, about 5, about 6, about 7, about 8, about 9, about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 370, about 380, about 390, about 400, about 450, or about 500 times.

In some embodiments, the RLP comprises one or more partial repeats. In some embodiments, the length of a partial repeat is 1, 2, 3, 4, 5, 6, 7, 8, 9, or 10 amino acids. In some embodiments, the RLP comprises one or more additional amino acids at the N-terminus or C-terminus of the RLP that are not part of a repeat.

In some embodiments, one or more RLP repeats are scrambled, i.e., they contain a different amino acid sequence but retain the same amino acid composition. For example, a repeat may have a different amino acid sequence than SEQ ID NO: 8, but retain the same amino acid composition.

In some embodiments, the polypeptide with phase behavior is an elastin-like polypeptide. Elastin-like polypeptides (ELPs) are biopolymers derived from tropoelastin. In some embodiments, the elastin-like polypeptides described herein are polymers comprising a pentapeptide repeat having the sequence (Val-Pro-Gly-Xaa-Gly)ₙ (SEQ ID NO: 10), wherein Xaa is defined herein.

In some embodiments, n is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500, including all values and ranges in between.

In some embodiments, the pentapeptide repeat is scrambled, for example it comprises a different amino acid sequence but maintains the same amino acid composition. For example, an ELP may comprise a different amino acid sequence than SEQ ID NO: 10, but maintains the same amino acid composition, e.g. 40% of the sequence is glycine, 20% of the sequence is Xaa (e.g., any amino acid except proline), 20% of the sequence is proline, and 20% of the sequence is valine.

In some embodiments, the ELP comprises one or more partial repeats. In some embodiments, the length of a partial repeat is 1, 2, 3, or 4 amino acids. In some embodiments, the ELP comprises one or more additional amino acids at the N-terminus or C-terminus of the ELP that are not part of a repeat.

In some embodiments, an ELP or RLP comprises from 30 to about 150 amino acids. In some embodiments, an ELP or RLP comprises from about 50 to about 100 amino acids. In embodiments, the ELP or RLP comprises about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 101, about 102, about 103, about 104, about 105, about 106, about 107, about 108, about 109, about 110, about 111, about 112, about 113, about 114, about 115, about 116, about 117, about 118, about 119, about 120, about 121, about 122, about 123, about 124, about 125, about 126, about 127, about 128, about 129, about 130, about 131, about 132, about 133, about 134, about 135, about 136, about 137, about 138, about 139, about 140, about 141, about 142, about 143, about 144, about 145, about 146, about 147, about 148, about 149, or about 150 amino acids. In embodiments, the ELP or RLP comprises at least about 30, at least about 31, at least about 32, at least about 33, at least about 34, at least about 35, at least about 36, at least about 37, at least about 38, at least about 39, at least about 40, at least about 41, at least about 42, at least about 43, at least about 44, at least about 45, at least about 46, at least about 47, at least about 48, at least about 49, at least about 50, at least about 51, at least about 52, at least about 53, at least about 54, at least about 55, at least about 56, at least about 57, at least about 58, at least about 59, at least about 60, at least about 61, at least about 62, at least about 63, at least about 64, at least about 65, at least about 66, at least about 67, at least about 68, at least about 69, at least about 70, at least about 71, at least about 72, at least about 73, at least about 74, at least about 75, at least about 76, at least about 77, at least about 78, at least about 79, at least about 80, at least about 81, at least about 82, at least about 83, at least about 84, at least about 85, at least about 86, at least about 87, at least about 88, at least about 89, at least about 90, at least about 91, at least about 92, at least about 93, at least about 94, at least about 95, at least about 96, at least about 97, at least about 98, at least about 99, at least about 100, at least about 101, at least about 102, at least about 103, at least about 104, at least about 105, at least about 106, at least about 107, at least about 108, at least about 109, at least about 110, at least about 111, at least about 112, at least about 113, at least about 114, at least about 115, at least about 116, at least about 117, at least about 118, at least about 119, at least about 120, at least about 121, at least about 122, at least about 123, at least about 124, at least about 125, at least about 126, at least about 127, at least about 128, at least about 129, at least about 130, at least about 131, at least about 132, at least about 133, at least about 134, at least about 135, at least about 136, at least about 137, at least about 138, at least about 139, at least about 140, at least about 141, at least about 142, at least about 143, at least about 144, at least about 145, at least about 146, at least about 147, at least about 148, at least about 149, or at least about 150 amino acids. In embodiments, the polypeptide with phase behavior may comprise any 30 to 150 amino acid fragment or any 50 to 100 amino acid fragment of an ELP or RLP described herein.

ELPs and RLPs undergo a phase transition in response to an environmental factor. ELPs and RLPs retain their ability to undergo a phase transition when coupled to one or more polypeptides (such as one or more AAV binding polypeptides), or expressed as a fusion protein with one or more other polypeptides (such as one or more AAV binding polypeptides). Polymers like ELPs and RLPs exhibit a transition temperature (Tₜ), also referred to as a cloud point temperature (Tc). In some embodiments ELPs and RLPs undergo a reversible phase transition from a soluble to an insoluble phase at the Tₜ. ELPs that transition from a soluble to an insoluble phase with heating or an increase in salt concentration have a Tₜ referred to as a lower critical solution temperature (LCST). RLPs that transition from a soluble to an insoluble phase with cooling or a decrease in salt concentration have a Tₜ referred to as a lower critical solution temperature (UCST). In some embodiments, the phase transition results from a change in secondary structure of the ELP and/or RLP. For example, the phase transition of an ELP results from a change in secondary structure from a random coil (below the Tₜ) to a type II β-turn. In some embodiments, the change in secondary structure is characterized by a method selected from circular dichroism spectropolarimetry, small angle x-ray scattering, ultraviolet-visible spectrophotometry, static light scattering, dynamic light scattering, nuclear magnetic resonance spectroscopy, solid-state nuclear magnetic resonance spectroscopy, infrared spectroscopy, Fourier transform infrared spectroscopy (FTIR), small angle neutron scattering, microscopy, and cryo-electron microscopy. In some embodiments, the phase transition of an ELP does not result from a change in secondary structure.

In some embodiments, the RLPs and ELPs described herein have a transition temperature between about 0° C. and about 100° C. In some embodiments, the RLPs and/or ELPs described herein have a transition temperature between about 10° C. and about 50° C. In some embodiments the transition temperature is about 0° C., about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., about 80° C., about 81° C., about 82° C., about 83° C., about 84° C., about 85° C., about 86° C., about 87° C., about 88° C., about 89° C., about 90° C., about 91° C., about 92° C., about 93° C., about 94° C., about 95° C., about 96° C., about 97° C., about 98° C., about 99° C., or about 100° C. In some embodiments the transition temperature is at least about 0° C., at least about 1° C., at least about 2° C., at least about 3° C., at least about 4° C., at least about 5° C., at least about 6° C., at least about 7° C., at least about 8° C., at least about 9° C., at least about 10° C., at least about 11° C., at least about 12° C., at least about 13° C., at least about 14° C., at least about 15° C., at least about 16° C., at least about 17° C., at least about 18° C., at least about 19° C., at least about 20° C., at least about 21° C., at least about 22° C., at least about 23° C., at least about 24° C., at least about 25° C., at least about 26° C., at least about 27° C., at least about 28° C., at least about 29° C., at least about 30° C., at least about 31° C., at least about 32° C., at least about 33° C., at least about 34° C., at least about 35° C., at least about 36° C., at least about 37° C., at least about 38° C., at least about 39° C., at least about 40° C., at least about 41° C., at least about 42° C., at least about 43° C., at least about 44° C., at least about 45° C., at least about 46° C., at least about 47° C., at least about 48° C., at least about 49° C., at least about 50° C., at least about 51° C., at least about 52° C., at least about 53° C., at least about 54° C., at least about 55° C., at least about 56° C., at least about 57° C., at least about 58° C., at least about 59° C., at least about 60° C., at least about 61° C., at least about 62° C., at least about 63° C., at least about 64° C., at least about 65° C., at least about 66° C., at least about 67° C., at least about 68° C., at least about 69° C., at least about 70° C., at least about 71° C., at least about 72° C., at least about 73° C., at least about 74° C., at least about 75° C., at least about 76° C., at least about 77° C., at least about 78° C., at least about 79° C., at least about 80° C., at least about 81° C., at least about 82° C., at least about 83° C., at least about 84° C., at least about 85° C., at least about 86° C., at least about 87° C., at least about 88° C., at least about 89° C., at least about 90° C., at least about 91° C., at least about 92° C., at least about 93° C., at least about 94° C., at least about 95° C., at least about 96° C., at least about 97° C., at least about 98° C., at least about 99° C., or at least about 100° C. In some embodiments, the RLPs described herein have a transition temperature from about 10° C. to about 100° C.

In some embodiments, the Tₜ of the RLPs and ELPs described herein is modulated by manipulating the primary structure (e.g. amino acid sequence) of the RLP and ELP. In some embodiments, the hydrophobicity of the ELP or RLP is modulated. In some embodiments, the hydrophobicity of the ELP is modified by altering the identity of the guest residue Xaa. In some embodiments, the hydrophobicity of the ELP or RLP is increased resulting in a decreased Tₜ. In some embodiments, the hydrophobicity of the ELP or RLP is decreased resulting in an increased Tₜ. In some embodiments, the polarity of the ELP or RLP is modulated. In some embodiments, the polarity of the ELP is modulated by altering the identity of the guest residue Xaa. In some embodiments, the polarity of the ELP or RLP is increased resulting in an increased Tₜ. In some embodiments, the polarity of the ELP or RLP is decreased resulting in a decreased Tₜ.

In some embodiments, the number of ELP pentapeptide repeats (n) is modulated to alter the Tₜ. In some embodiments, n of the pentapeptide repeat (Val-Pro-Gly-Xaa-Gly)ₙ (SEQ ID NO: 10) is an integer from 1 to 500, inclusive of endpoints. In some embodiments, n is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500, including all values and ranges in between.

In some embodiments, Xaa is a “guest residue,” i.e., any amino acid that does not eliminate the phase behavior of the ELP. In some embodiments, Xaa is any amino acid except proline. In some embodiments, Xaa is independently selected for each repeat. For example, a given ELP may comprise the guest residues alanine, glycine, and valine at a ratio of 8:7:1. In some embodiments Xaa is selected from the group consisting of alanine, arginine, asparagine, aspartic acid, cysteine, glutamic acid, glutamine, glycine, histidine, isoleucine, leucine, lysine, methionine, phenylalanine, praline, serine, threonine, tryptophan, tyrosine and valine. In some embodiments, Xaa is a non-classical amino acid selected from Table 2 and/or the group consisting of 2,4-diaminobutyric acid, α-amino-isobutyric acid, alloisoleucine, 4-aminobutyric acid, 2-amino butyric acid (Abu), ε-Ahx, 6-amino hexanoic acid, 2-amino isobutyric acid (Aib), 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Na-methyl amino acids, and amino acid analogs in general. In some embodiments, Xaa is the D-isomer of a natural or non-classical amino acid.

In some embodiments, the Tₜ of the RLPs and ELPs described herein is modulated by introducing one or more environmental factors to the composition comprising the RLP and/or ELP. In some embodiments, the Tₜ of the ELPs and/or RLPs is modulated by adjusting the ionic strength of solvents. In some embodiments, the ionic strength of the solvent is adjusted by adding salt. In some embodiments, ELPs and/or RLPs comprise lower Tₜ in solvents comprising anions categorized as kosmotropes. Anions that are kosmotropes are highly hydrated and influence the water shield on ELPs and/or RLPs. In some embodiments, the Tₜ of ELPs and/or RLPs can be adjusted through the addition of anions that are chaotropes. At low concentrations, the addition of chaotropes increase the Tₜ of the ELP and/or RLP. At high concentrations, the addition of a chaotrope decreases the Tₜ of the ELP and/or RLP. In some embodiments, the Tₜ of the ELP and/or RLP can be tuned by introducing one or more reagents that disrupts hydrogen bonds. Non-limiting examples of reagents that disrupt hydrogen bonds include sodium dodecyl sulfate (SDS) and urea. In some embodiments, reagents that enhance hydrogen bond formation are utilized to modulate the Tₜ. In some embodiments, reagents that enhance hydrophobic interactions are utilized to modulate the Tₜ. Trifluoroethanol is a reagent which enhances both hydrophobic interactions and hydrogen bond formation, causing a decrease in Tₜ.

In some embodiments, the ELP and/or RLP concentration can be adjusted to modulate Tₜ. In some embodiments, a higher ELP and/or RLP concentration results in a reduced Tₜ. In some embodiments, a lower ELP and/or RLP concentration results in an increased Tₜ.

In addition, modulation of pH, light, and ion concentrations also can be utilized to modulate Tₜ.

In some embodiments, modulation of the number of (e.g. addition or removal) charged amino acids (e.g. histidine, lysine, arginine, glutamic acid, aspartic acid, ornithine, or other non-natural charged amino acids) and identity (e.g. positively or negatively charged) enables tuning of the Tₜ through pH modulation.

In some embodiments, the ELPs and/or RLPs described herein are block copolymers. A block copolymer comprises two or more sequence domains or blocks, in which two or more blocks comprise different properties. Non-limiting examples of properties that can be tuned include hydrophilicity, hydrophobicity, polarity, and secondary structure. In some embodiments, the block copolymer is an amphiphile, e.g. it comprises at least one hydrophobic and at least one hydrophilic block.

In some embodiments, the ELPs and/or RLPs described herein assemble into various morphologies. Non-limiting examples of morphologies include a spherical aggregate, a micelle, a vesicle, a fibril, a nanofibril, a nanotube, and a hydrogel. In some embodiments, the RLPs and/or ELPs described herein assemble into various morphologies after the addition of an environmental factor. In some embodiments, the RLPs and/or ELPs described herein change from one morphology to another morphology after the addition of an environmental factor. In some embodiments, the RLPs and/or ELPs described herein change from one morphology to another morphology after the addition of an AAV particle.

In some embodiments, addition of an environmental factor causes an RLP and/or ELP to undergo a phase transition. In some embodiments, at the RLP and/or ELP phase transition, the RLP and/or ELP converts from one morphology to another morphology.

In some embodiments, a phase transition of an RLP and/or ELP causes the formation of dense, liquid, droplets.

In some embodiments, the polypeptide with phase behavior comprises an amino acid sequence selected from the group consisting of:

(a)
(SEQ ID NO: 1)
(GRGDSPY)ₙ
(b)
(SEQ ID NO: 2)
(GRGDSPH)ₙ
(c)
(SEQ ID NO: 3)
(GRGDSPV)ₙ
(d)
(SEQ ID NO: 4)
(GRGDSPYG)ₙ
(e)
(SEQ ID NO: 5)
(RPLGYDS)ₙ
(f)
(SEQ ID NO: 6)
(RPAGYDS)ₙ
(g)
(SEQ ID NO: 7)
(GRGDSYP)ₙ
(h)
(SEQ ID NO: 8)
(GRGDSPYQ)ₙ
(i)
(SEQ ID NO: 9)
(GRGNSPYG)ₙ
(j)
(SEQ ID NO: 11)
(GVGVP)ₙ;
(k)
(SEQ ID NO: 12)
(GVGVPGLGVPGVGVPGLGVPGVGVP)ₘ;
(l)
(SEQ ID NO: 13)
(GVGVPGVGVPGAGVPGVGVPGVGVP)ₘ;
(m)
(SEQ ID NO: 14)
(GVGVPGWGVPGVGVPGWGVPGVGVP)ₘ;
(n)
(SEQ ID NO: 15)
(GVGVPGVGVPGVGVPGVGVPGVGVPGV
GVPGEGVPGFGVPGVGVP)ₘ;
(o)
(SEQ ID NO: 16)
(GVGVPGVGVPGVGVPGVGVPGVGVPGVG
VPGKGVPGFGVPGVGVP)ₘ;
and
(p)
(SEQ ID NO: 17)
(GAGVPGVGVPGAGVPGVGVPGAGVP)ₘ;

or a randomized, scrambled analog thereof;
wherein:
n is an integer in the range of 1-500, inclusive of endpoints; and
m is an integer in the range of 4-25, inclusive of endpoints.

In some embodiments, the polypeptide with phase behavior is (GVGVPGLGVPGVGVPGLGVPGVGVP)ₘ (SEQ ID NO: 12), wherein m is 16. In some embodiments, the polypeptide with phase behavior comprises an amino acid sequence of (GVGVPGLGVPGVGVPGLGVPGVGVP)ₘ (SEQ ID NO: 12), wherein m is 16, and up to 10 additional amino acids at the N-terminus or C-terminus. In some embodiments, the polypeptide with phase behavior comprises an amino acid sequence of (GVGVPGLGVPGVGVPGLGVPGVGVP)ₘ (SEQ ID NO: 12), wherein m is 16, and an additional C-terminal glycine. In some embodiments, the polypeptide with phase behavior comprises an amino acid sequence of (GVGVPGLGVPGVGVPGLGVPGVGVP)ₘ (SEQ ID NO: 12), wherein m is 16, and an additional N-terminal methionine. In some embodiments, the polypeptide with phase behavior comprises an amino acid sequence of (GVGVPGLGVPGVGVPGLGVPGVGVP)ₘ (SEQ ID NO: 12), wherein m is 16, and an additional C-terminal glycine and an additional N-terminal methionine.

In some embodiments, the polypeptide with phase behavior has an amino acid sequence of SEQ ID NO: 88.

In some embodiments, the polypeptide with phase behavior comprises an amino acid sequence of (GVGVPGVGVPGAGVPGVGVPGVGVP)ₘ (SEQ ID NO: 144) or (GVGVPGVGVPGLGVPGVGVPGVGVP)ₘ (SEQ ID NO: 146), wherein m is an integer between 2 and 32, inclusive of endpoints. In some embodiments, the polypeptide with phase behavior comprises an amino acid sequence of (GVGVPGVGVPGAGVPGVGVPGVGVP)ₘ (SEQ ID NO: 144), wherein m is 8 or 16. In some embodiments, the polypeptide with phase behavior comprises an amino acid sequence of (GVGVPGAGVP)ₘ (SEQ ID NO: 145), wherein m is an integer between 5 and 80, inclusive of endpoints. In some embodiments, the polypeptide with phase behavior comprises an amino acid sequence of (GXGVP)ₘ (SEQ ID NO: 147), wherein m is an integer between 10 and 160, inclusive of endpoints, and wherein X for each repeat is independently selected from the group consisting of glycine, alanine, valine, isoleucine, leucine, phenylalanine, tyrosine, tryptophan, lysine, arginine, aspartic acid, glutamic acid, and serine.

In some embodiments, the polypeptide with phase behavior comprises an amino acid sequence selected from

(a)
(SEQ ID NO: 143)
(GVGVP)ₘ;
(b)
(SEQ ID NO: 148)
(ZZPXXXXGZ)ₘ;
(c)
(SEQ ID NO: 149)
(ZZPXGZ)ₘ;
(d)
(SEQ ID NO: 150)
(ZZPXXGZ)ₘ;
or
(e)
(SEQ ID NO: 151)
(ZZPXXXGZ)ₘ,

wherein m is an integer between 10 and 160, inclusive of endpoints, wherein X if present is any amino acid except proline or glycine, and wherein Z if present is any amino acid.

In some embodiments, the polypeptide with phase behavior comprises an amino acid sequence of (GVGVP)ₘ (SEQ ID NO: 143), wherein m is 20, 40, or 80. In some embodiments, the polypeptide with phase behavior comprises an amino acid sequence of (GRGDXPZX)ₘ (SEQ ID NO: 152) or (XZPXDGRG)ₘ (SEQ ID NO: 153), wherein X is glutamine or serine, Z is tyrosine or valine, and m is an integer between 10 and 160, inclusive of endpoints.

In some embodiments, the polypeptide with phase behavior comprises a first set of repeat sequences and a second set of repeat sequences. The first set of repeat sequences and the second set of repeat sequences may each individually comprise sequences that are repeated one or more times. In some embodiments, the first set of repeat sequences any/or the second set of repeat sequences comprises a repeating sequence comprising any one of SEQ ID NOs: 1-17 and 143-153. In some embodiments, the polypeptide with phase behavior comprises a first set of repeat sequences and a second set of repeat sequences, wherein the first set of repeat sequences comprises the amino acid sequence of (GRGDXPZX)₄₀ (SEQ ID NO: 185) and the second set of repeat sequences comprises the amino acid sequence (GVGVP)₈₀ (SEQ ID NO: 186), wherein X is glutamine and Z is tyrosine. In some embodiments, the first set of repeat sequences comprises the sequence of SEQ ID NO: 187. In some embodiments, the polypeptide with phase behavior comprises at least one, at least two, at least three, at least four, at least five, at least six, at least seven, at least eight, at least nine, or at least ten different sets of repeat sequences. In some embodiments, each set of repeat sequences within the polypeptide with phase behavior comprises sequences that repeat from about 5 to about 400 times, for example, about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335, about 340, about 345, about 350, about 355, about 360, about 365, about 370, about 375, about 380, about 385, about 390, about 395, or about 400 times.

In some embodiments, the polypeptide with phase behavior comprising an amino acid sequence selected from any one of SEQ ID NOs: 1-17, 88, and 143-153 also comprises up to 10 additional N-terminal and/or C-terminal amino acids. In some embodiments, the polypeptide with phase behavior comprising an amino acid sequence of any one of SEQ ID NOs: 1-17, 88, and 143-153 also comprises an additional N-terminal methionine. In some embodiments, the polypeptide with phase behavior comprising an amino acid sequence of any one of SEQ ID NOs: 1-17, 88, and 143-153 also comprises an additional C-terminal glycine.

In some embodiments, the polypeptide with phase behavior has the same amino acid composition of an ELP and/or RLP but does not comprise repeats. In some embodiments, the polypeptide with phase behavior comprises an amino acid sequence that is about 80%, about 85%, about 90%, about 95%, about 96%, about 97%, about 98%, about 99%, or about 100% identical to an ELP and/or RLP. In some embodiments, the polypeptide with phase behavior comprises an amino acid composition that is about 80%, about 85%, about 90%, about 95%, about 96%, about 97%, about 98%, about 99%, or about 100% identical to an ELP and/or RLP. In some embodiments, the polypeptide with phase behavior comprises a composition of hydrophobic amino acids that is about 80%, about 85%, about 90%, about 95%, about 96%, about 97%, about 98%, about 99%, or about 100% identical to an ELP and/or RLP.

In some embodiments, the polypeptide with phase behavior comprises a non-repetitive unstructured polypeptide. In some embodiments, the non-repetitive unstructured polypeptide has an amino acid sequence that comprises at least 50 amino acids. In some embodiments, the non-repetitive unstructured polypeptide has an amino acid sequence that comprises at least 50, at least 60, at least 70, at least 80, at least 90, or at least 100 amino acids. In some embodiments, the sequence of the non-repetitive unstructured polypeptide is at least about 10% proline (e.g., at least 10%, at least 20%, at least 30%, at least 40%, at least 50%, at least 60%, at least 70%, or at least 80%) and at least 20% glycine (e.g. at least 20%, at least 30%, at least 40%, at least 50%, at least 60%, at least 70%, at least 80%, or at least 90%). In some embodiments, the non-repetitive unstructured polypeptide has a sequence that comprises at least about 40% of amino acids selected from the group consisting of valine, alanine, leucine, lysine, threonine, isoleucine, tyrosine, serine, and phenylalanine.

In some embodiments, the polypeptide with phase behavior comprises a sequence that does not comprise three contiguous identical amino acids, wherein any 5-10 amino acid subsequence does not occur more than once in the polypeptide with phase behavior, and wherein when the polypeptide with phase behavior comprises a subsequence starting and ending with proline, and wherein the subsequence further comprises at least one glycine.

The ELPs and/or RLPs described herein are expressed as a component of a fusion protein, e.g., the second polypeptide, wherein the second polypeptide has phase behavior. In some embodiments, a fusion protein comprises a first polypeptide. Examples of first polypeptides are provided throughout this example. In some embodiments, the first polypeptide is (i) an enzyme, or a derivative or catalytic fragment thereof; (ii) an antibody, or a derivative or antigen-binding fragment thereof; (iii) a signaling molecule, or a fragment or derivative thereof; (iv) a structural protein, or a fragment or derivative thereof; or (v) a hormone, or a fragment or derivative thereof.

In some embodiments, the fusion protein is expressed in bacteria or mammalian cells.

In some embodiments, the fusion protein is expressed in Escherichia coli. In some embodiments, the fusion protein is expressed in insect cells. In some embodiments, the sequence of the non-repetitive unstructured polypeptide is at least about 10% proline (e.g. at least 10%, at least 20%, at least 30%, at least 40%) and at least 20% glycine (e.g. at least 20%, at least 30%, at least 40%, or at least 50%), and at least 40% (e.g. at least 40%, at least 50%, at least 60%, or at least 70%) of amino acids selected from the group consisting of valine, alanine, leucine, lysine, threonine, isoleucine, tyrosine, serine, and phenylalanine.

In some embodiments, the polypeptide with phase behavior does not comprise three contiguous identical amino acids. In some embodiments, the polypeptide with phase behavior comprises a subsequence (e.g., a fragment of the polypeptide with phase behavior) which only occurs once in the amino acid sequence of the polypeptide with phase behavior. In some embodiments, the polypeptide with phase behavior comprises a subsequence that starts and ends with proline. In some embodiments, the polypeptide with phase behavior comprises a subsequence that comprises at least one glycine.

In some embodiments, the polypeptide with phase behavior comprises a signal peptide. In some embodiments, the polypeptide with phase behavior comprises an N-terminal methionine. In some embodiments, the polypeptide with phase behavior lacks an N-terminal methionine. As used herein, “(M)” refers to an optional N-terminal methionine.

### Linkers

In some embodiments, a linker separates the first polypeptide and the second polypeptide of the fusion protein. In some embodiments, any linker that does not interfere with the function of the fusion protein may be utilized. In some embodiments, the linker may be flexible. In some embodiments, the linker may be rigid.

In some embodiments, the linker preserves the phase behavior of the polypeptide with phase behavior. In some embodiments, the linker preserves the Tₜ of the polypeptide with phase behavior. In some embodiments, the linker preserves the structure of the first polypeptide. In some embodiments, the linker comprises between 1 and 50 amino acids. In some embodiments, the linker comprises about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids.

In some embodiments, the stiffness of the linker is increased by the inclusion of proline in the linker amino acid sequence.

In some embodiments, the flexibility of a linker is increased by the inclusion of small polar amino acids, including threonine, serine, and glycine.

In some embodiments, the linker may adopt various secondary structures, including but not limited to α-helices, β-strands, and random coils. In some embodiments, the linker adopts an α-helix and comprises an amino acid repeat of (EAAAK)ₙ (SEQ ID NO: 18) where n is a repeat number from 1 to 20.

In some embodiments, the linker is comprised of (G₄S)ₙ (SEQ ID NO: 19) where n can be a repeat number from 1 to 30 (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30). In embodiments, the linker has a repeat of (SGGG)n (SEQ ID NO: 20), wherein n is an integer from 1 to 50 (e.g. 1, 2, 3, 4, 5, 6, 7, 8, 9, 10, 11, 12, 13, 14, 15, 16, 17, 18, 19, 20). In embodiments, the linker has a repeat of (GGGS)ₙ (SEQ ID NO: 21), wherein n is an integer from 1 to 20 (e.g. 1, 2, 3, 4, 5, 6, 7, 8, 9, 10, 11, 12, 13, 14, 15, 16, 17, 18, 19, or 20). In some embodiments, the linker has a repeat of (GₓS)ₙ (SEQ ID NO: 141), wherein x is an integer from 1 to 6 (e.g. 1, 2, 3, 4, 5, or 6), and n is an integer from 1 to 30 (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30). In some embodiments, the linker has a repeat of (SₓG)ₙ (SEQ ID NO: 142), wherein x is an integer from 1 to 6 (e.g. 1, 2, 3, 4, 5, or 6), and n is an integer from 1 to 30 (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30).

In some embodiments, the linker has an amino acid sequence of KESGSVSSEQLAQFRSLD (SEQ ID NO: 22). In some embodiments, the linker has an amino acid sequence of EGKSSGSGSESKST (SEQ ID NO: 23). In some embodiments, the linker only comprises glycine.

In some embodiments, the linker is cleavable. For example, the linker may be cleaved by a protease. In some embodiments, the peptide linker comprises a protease cleavage site. In some embodiments, the protease cleavage site is a furin cleavage site.

In some aspects, the linker is a poly-(Gly)ₙ linker, wherein n is 1, 2, 3, 4, 5, 6, 7, 8, 9, 10, 15, 16, 17, 18, 19, 20, 21, 22, 23, 24, 25, 26, 27, 28, 29, or 30 (SEQ ID NO: 48). In other embodiments, the linker is selected from the group consisting of: dipeptides, tripeptides, and quadripeptides. In embodiments, the linker is a dipeptide selected from the group consisting of alanine-serine (AS), leucine-glutamic acid (LE), and serine-arginine (SR).

In some embodiments, the linker is selected from GKSSGSGSESKS (SEQ ID NO: 157), GSTSGSGKSSEGKG (SEQ ID NO: 158), GSTSGSGKSSEGSGSTKG (SEQ ID NO: 159), GSTSGSGKPGSGEGSTKG (SEQ ID NO: 160), EGKSSGSGSESKEF (SEQ ID NO: 161), SRSSG (SEQ ID NO: 162), and SGSSC (SEQ ID NO: 163).

In some embodiments, the linker is a self-cleaving peptide. In some embodiments, the self-cleaving peptide is a 2A peptide. 2A peptides are a class of 18-22 amino acid long peptides that induce ribosomal skipping during translation of a protein in a cell. In some embodiments, the 2A peptide is a T2A peptide having an amino acid sequence of EGRGSLLTCGDVEENPGP (SEQ ID NO: 164), a P2A peptide having an amino acid sequence of ATNFSLLKQAGDVEENPGP (SEQ ID NO: 165), an E2A peptide having an amino acid sequence of QCTNYALLKLAGDVESNPGP (SEQ ID NO: 166), or an F2A peptide having an amino acid sequence of VKQTLNFDLLKLAGDVESNPGP (SEQ ID NO: 167). In some embodiments, the 2A peptide has at least 80%, at least 85%, at least 90%, at least 95%, or at least 98% identity to any one of SEQ ID NOs. 164-167. In some embodiments, the 2A peptide further comprises GSG (SEQ ID NO: 168) on its N-terminus.

In some embodiments, a fusion protein comprises, from N-terminus to C-terminus, a first polypeptide, a linker, and a second polypeptide with phase behavior. In some embodiments, a fusion protein comprises, from N-terminus to C-terminus, a second polypeptide with phase behavior, a linker, and a first polypeptide.

Methods for Stabilizing, Improving Yield, and Preventing Loss of Activity, Unfolding, Degradation, and/or Misfolding the First Polypeptide of a Fusion Protein

The instant inventors have discovered that expression of a first polypeptide as a fusion protein comprising the first polypeptide and a second polypeptide with phase behavior may unexpectedly help stabilize the first polypeptide during production, purification, and/or storage thereof. As used herein with relation to the first polypeptide, the terms “stabilize” or “stabilizing” refers to the ability of expression as a fusion protein comprising a first polypeptide and a second polypeptide with phase behavior to reduce degradation or aggregation of a sample comprising a plurality of first polypeptides, to prevent the first polypeptides from binding other proteins or to themselves, to enhance synthesis of a first polypeptide by a producer cell, to prevent unfolding of a first polypeptide, and to prevent misfolding of a first polypeptide.

Thus, in some embodiments, a method of stabilizing a first polypeptide is provided herein comprising expressing the first polypeptide as a fusion protein comprising the first polypeptide and a second polypeptide, wherein the second polypeptide is a polypeptide having phase behavior. In embodiments, the first polypeptide substantially retains its activity after the fusion protein is exposed to one or more conditions that would destabilize the first polypeptide. In some embodiments, a method of substantially preventing the unfolding, degradation and/or misfolding of the first polypeptide is provided herein comprising expressing a first polypeptide as a fusion protein of a first polypeptide and a second polypeptide having phase behavior, wherein when the fusion protein is exposed to one or more conditions that would cause unfolding, degradation, and/or misfolding. In some embodiments, a method for substantially preventing loss of activity of a first polypeptide after exposure to one or more conditions known to unfold, degrade, and/or misfold the first polypeptide is provided herein comprising expressing a fusion protein comprising the first polypeptide and the second polypeptide, wherein the second polypeptide is a polypeptide having phase behavior. In embodiments, any of the aforementioned methods comprises removing the fusion protein from the conditions known to destabilize the first polypeptide or conditions known to unfold, degrade, and/or misfold the first polypeptide; wherein the first polypeptide retains its activity compared to a control first polypeptide. The “control first polypeptide” refers to a first polypeptide that is not fused to a polypeptide with phase behavior and which has not been exposed to conditions that cause loss of activity, misfolding, unfolding, or degradation.

The term “activity” may refer to the binding affinity of a first polypeptide for its binding partner. For example, if the first polypeptide is PKD2 of the AAVR, the binding partner may be the capsid of an AAV viral particle. If the first polypeptide is protein A, the binding partner may be the Fc region of an immunoglobulin. When referring to an enzyme, the term “activity” may refer to the k_{cat}, also referred to herein as “turnover number.” k_{cat} is calculated using the formula Vₘₐₓ/Eₜ, where Vₘₐₓ is the maximum rate of reaction when all the enzyme catalytic sites are saturated with substrate and Eₜ is the total enzyme concentration or concentration of total enzyme catalytic sites. Multiple techniques may be utilized to determine enzyme kinetic parameters like “k_{cat}” and “Vₘₐₓ,” for example, surface plasmon resonance, Forster Resonance Energy Transfer (FRET), isothermal titration calorimetry, colorimetric, or fluorometric techniques.

Conditions known to destabilize, unfold, degrade, or misfold a first polypeptide include the introduction of one or more of the following: salt, exposure to a base (e.g., a Bronsted-Lowry base or Lewis base), exposure to an acid (e.g., a Bronsted-Lowry acid or Lewis acid), exposure to a temperature of at least 50° C., lyophilization, freeze-thaw cycles, autoclave, an oxidizing agent, a reducing agent, a chaotropic agent, a surfactant, an organic solvent, urea, or guanidine hydrochloride. In some embodiments, autoclaving, heat shock, a change in pH, exposure to light, agitation, mixing, a change in temperature (e.g., a freeze-thaw), storing a first polypeptide in a non-ideal orientation, or a tendency of the first polypeptide to aggregate cause unfolding, degradation, or misfolding of the first polypeptide. In embodiments, any of the environmental factors described herein may be a condition that destabilizes, unfolds, degrades, or misfolds a first polypeptide.

In embodiments, the conditions known to unfold, degrade, misfold, or destabilize the first polypeptide are exposure to guanidine hydrochloride, lyophilization, freeze-thaw cycles, exposure to sodium hydroxide, autoclaving, exposure to temperature of at least 50° C., or a combination thereof.

In embodiments, the fusion protein is exposed to one or more conditions known to unfold, degrade, misfold, or destabilize the first polypeptide for about 5 minutes to about 1 day. In embodiments, the first polypeptide is exposed to one or more conditions known to unfold, degrade, misfold, or destabilize the first polypeptide for about 10 minutes to about 30 minutes, about 15 minutes to about 30 minutes, about 15 minutes to 1 hour, about 30 minutes to about 12 hours, about 30 minutes to about 11 hours, about 30 minutes to about 10 hours, about 30 minutes to about 9 hours, about 30 minutes to about 8 hours, about 30 minutes to about 7 hours, about 30 minutes to about 6 hours, about 30 minutes to about 5 hours, about 30 minutes to about 4 hours, about 30 minutes to about 3 hours, about 30 minutes to about 2 hours, about 30 minutes to about 1 hour, or about 45 minutes to 1 hour. In embodiments the first polypeptide is exposed to a condition for about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, about 40 minutes, about 41 minutes, about 42 minutes, about 43 minutes, about 44 minutes, about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours, including all subranges and values therebetween. In embodiments the first polypeptide is exposed to a condition for at least about 1 minute, at least about 2 minutes, at least about 3 minutes, at least about 4 minutes, at least about 5 minutes, at least about 6 minutes, at least about 7 minutes, at least about 8 minutes, at least about 9 minutes, at least about 10 minutes, at least about 11 minutes, at least about 12 minutes, at least about 13 minutes, at least about 14 minutes, at least about 15 minutes, at least about 16 minutes, at least about 17 minutes, at least about 18 minutes, at least about 19 minutes, at least about 20 minutes, at least about 21 minutes, at least about 22 minutes, at least about 23 minutes, at least about 24 minutes, at least about 25 minutes, at least about 26 minutes, at least about 27 minutes, at least about 28 minutes, at least about 29 minutes, at least about 30 minutes, at least about 31 minutes, at least about 32 minutes, at least about 33 minutes, at least about 34 minutes, at least about 35 minutes, at least about 36 minutes, at least about 37 minutes, at least about 38 minutes, at least about 39 minutes, at least about 40 minutes, at least about 41 minutes, at least about 42 minutes, at least about 43 minutes, at least about 44 minutes, at least about 45 minutes, at least about 46 minutes, at least about 47 minutes, at least about 48 minutes, at least about 49 minutes, at least about 50 minutes, at least about 51 minutes, at least about 52 minutes, at least about 53 minutes, at least about 54 minutes, at least about 55 minutes, at least about 56 minutes, at least about 57 minutes, at least about 58 minutes, at least about 59 minutes, at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, or at least about 24 hours. In embodiments, the first polypeptide is exposed to one or more conditions known to unfold, degrade, misfold, or destabilize the first polypeptide for about 30 minutes. In embodiments, the first polypeptide is exposed to one or more conditions known to unfold, degrade, misfold, or destabilize the first polypeptide for at least about 30 minutes. In embodiments, the first polypeptide is exposed to one or more conditions known to unfold, degrade, misfold, or destabilize the first polypeptide for about 10 minutes to about 30 minutes.

In embodiments, the fusion protein is exposed to a temperature ranging from about 50° C. to about 99° C., e.g., about 50° C., about 55° C., about 60° C., about 65° C., about 70° C., about 75° C., about 80° C., about 85° C., about 90° C., about 91° C., about 92° C., about 93° C., about 94° C., about 95° C., about 96° C., about 97° C., about 98° C., or about 99° C. including all subranges and values therebetween. In embodiments, the fusion protein is exposed to at least about 50° C., at least about 55° C., at least about 60° C., at least about 65° C., at least about 70° C., at least about 75° C., at least about 80° C., at least about 85° C., at least about 90° C., at least about 91° C., at least about 92° C., at least about 93° C., at least about 94° C., at least about 95° C., at least about 96° C., at least about 97° C., at least about 98° C., or at least about 99° C.

In embodiments, the condition known to destabilize, unfold, degrade, or misfold a first polypeptide is exposure to guanidine hydrochloride. In embodiments, the first polypeptide is exposed to from 1 M to about 10 M guanidine hydrochloride. In embodiments, the first polypeptide is exposed to about 1 M, about 1.1 M, about 1.2 M, about 1.3 M, about 1.4 M, about 1.5 M, about 1.6 M, about 1.7 M, about 1.8 M, about 1.9 M, about 2 M, about 2.1 M, about 2.2 M, about 2.3 M, about 2.4 M, about 2.5 M, about 2.6 M, about 2.7 M, about 2.8 M, about 2.9 M, about 3 M, about 3.1 M, about 3.2 M, about 3.3 M, about 3.4 M, about 3.5 M, about 3.6 M, about 3.7 M, about 3.8 M, about 3.9 M, about 4 M, about 4.1 M, about 4.2 M, about 4.3 M, about 4.4 M, about 4.5 M, about 4.6 M, about 4.7 M, about 4.8 M, about 4.9 M, about 5 M, about 5.1 M, about 5.2 M, about 5.3 M, about 5.4 M, about 5.5 M, about 5.6 M, about 5.7 M, about 5.8 M, about 5.9 M, about 6 M, about 6.1 M, about 6.2 M, about 6.3 M, about 6.4 M, about 6.5 M, about 6.6 M, about 6.7 M, about 6.8 M, about 6.9 M, about 7 M, about 7.1 M, about 7.2 M, about 7.3 M, about 7.4 M, about 7.5 M, about 7.6 M, about 7.7 M, about 7.8 M, about 7.9 M, about 8 M, about 8.1 M, about 8.2 M, about 8.3 M, about 8.4 M, about 8.5 M, about 8.6 M, about 8.7 M, about 8.8 M, about 8.9 M, about 9 M, about 9.1 M, about 9.2 M, about 9.3 M, about 9.4 M, about 9.5 M, about 9.6 M, about 9.7 M, about 9.8 M, about 9.9 M, or about 10 M guanidine hydrochloride, including all subranges and values therebetween. In embodiments, the first polypeptide is exposed to at least about 1 M, at least about 1.1 M, at least about 1.2 M, at least about 1.3 M, at least about 1.4 M, at least about 1.5 M, at least about 1.6 M, at least about 1.7 M, at least about 1.8 M, at least about 1.9 M, at least about 2 M, at least about 2.1 M, at least about 2.2 M, at least about 2.3 M, at least about 2.4 M, at least about 2.5 M, at least about 2.6 M, at least about 2.7 M, at least about 2.8 M, at least about 2.9 M, at least about 3 M, at least about 3.1 M, at least about 3.2 M, at least about 3.3 M, at least about 3.4 M, at least about 3.5 M, at least about 3.6 M, at least about 3.7 M, at least about 3.8 M, at least about 3.9 M, at least about 4 M, at least about 4.1 M, at least about 4.2 M, at least about 4.3 M, at least about 4.4 M, at least about 4.5 M, at least about 4.6 M, at least about 4.7 M, at least about 4.8 M, at least about 4.9 M, at least about 5 M, at least about 5.1 M, at least about 5.2 M, at least about 5.3 M, at least about 5.4 M, at least about 5.5 M, at least about 5.6 M, at least about 5.7 M, at least about 5.8 M, at least about 5.9 M, at least about 6 M, at least about 6.1 M, at least about 6.2 M, at least about 6.3 M, at least about 6.4 M, at least about 6.5 M, at least about 6.6 M, at least about 6.7 M, at least about 6.8 M, at least about 6.9 M, at least about 7 M, at least about 7.1 M, at least about 7.2 M, at least about 7.3 M, at least about 7.4 M, at least about 7.5 M, at least about 7.6 M, at least about 7.7 M, at least about 7.8 M, at least about 7.9 M, at least about 8 M, at least about 8.1 M, at least about 8.2 M, at least about 8.3 M, at least about 8.4 M, at least about 8.5 M, at least about 8.6 M, at least about 8.7 M, at least about 8.8 M, at least about 8.9 M, at least about 9 M, at least about 9.1 M, at least about 9.2 M, at least about 9.3 M, at least about 9.4 M, at least about 9.5 M, at least about 9.6 M, at least about 9.7 M, at least about 9.8 M, at least about 9.9 M, or at least about 10 M guanidine hydrochloride. In embodiments, the condition known to destabilize, unfold, degrade, or misfold a first polypeptide is exposure to 6M guanidine hydrochloride. In embodiments, the condition known to destabilize, unfold, degrade, or misfold a first polypeptide is exposure to 6M guanidine hydrochloride for 30 minutes. In embodiments, the condition known to destabilize, unfold, degrade, or misfold a first polypeptide is exposure to 6M guanidine hydrochloride for at least 30 minutes. In embodiments, the condition known to destabilize, unfold, degrade, or misfold a first polypeptide is exposure to 6M guanidine hydrochloride for 10-30 minutes.

In embodiments, the condition known to destabilize, unfold, degrade, or misfold a first polypeptide is exposure to sodium hydroxide. In embodiments, the first polypeptide is exposed to from 0.001 M to about 10 M sodium hydroxide. In embodiments, the first polypeptide is exposed to about 0.001 M, about 0.01 M, about 0.02 M, about 0.03 M, about 0.04 M, about 0.05 M, about 0.06 M, about 0.07 M, about 0.08 M, about 0.09 M, about 0.1 M, about 0.2 M, about 0.3 M, about 0.4 M, about 0.5 M, about 0.6 M, about 0.7 M, about 0.8 M, about 0.9 M, about 1 M, about 1.1 M, about 1.2 M, about 1.3 M, about 1.4 M, about 1.5 M, about 1.6 M, about 1.7 M, about 1.8 M, about 1.9 M, about 2 M, about 2.1 M, about 2.2 M, about 2.3 M, about 2.4 M, about 2.5 M, about 2.6 M, about 2.7 M, about 2.8 M, about 2.9 M, about 3 M, about 3.1 M, about 3.2 M, about 3.3 M, about 3.4 M, about 3.5 M, about 3.6 M, about 3.7 M, about 3.8 M, about 3.9 M, about 4 M, about 4.1 M, about 4.2 M, about 4.3 M, about 4.4 M, about 4.5 M, about 4.6 M, about 4.7 M, about 4.8 M, about 4.9 M, about 5 M, about 5.1 M, about 5.2 M, about 5.3 M, about 5.4 M, about 5.5 M, about 5.6 M, about 5.7 M, about 5.8 M, about 5.9 M, about 6 M, about 6.1 M, about 6.2 M, about 6.3 M, about 6.4 M, about 6.5 M, about 6.6 M, about 6.7 M, about 6.8 M, about 6.9 M, about 7 M, about 7.1 M, about 7.2 M, about 7.3 M, about 7.4 M, about 7.5 M, about 7.6 M, about 7.7 M, about 7.8 M, about 7.9 M, about 8 M, about 8.1 M, about 8.2 M, about 8.3 M, about 8.4 M, about 8.5 M, about 8.6 M, about 8.7 M, about 8.8 M, about 8.9 M, about 9 M, about 9.1 M, about 9.2 M, about 9.3 M, about 9.4 M, about 9.5 M, about 9.6 M, about 9.7 M, about 9.8 M, about 9.9 M, or about 10 M sodium hydroxide, including all subranges and values therebetween. In embodiments, the first polypeptide is exposed to at least about 0.001 M, at least about 0.01 M, at least about 0.02 M, at least about 0.03 M, at least about 0.04 M, at least about 0.05 M, at least about 0.06 M, at least about 0.07 M, at least about 0.08 M, at least about 0.09 M, at least about 0.1 M, at least about 0.2 M, at least about 0.3 M, at least about 0.4 M, at least about 0.5 M, at least about 0.6 M, at least about 0.7 M, at least about 0.8 M, at least about 0.9 M, at least about 1 M, at least about 1.1 M, at least about 1.2 M, at least about 1.3 M, at least about 1.4 M, at least about 1.5 M, at least about 1.6 M, at least about 1.7 M, at least about 1.8 M, at least about 1.9 M, at least about 2 M, at least about 2.1 M, at least about 2.2 M, at least about 2.3 M, at least about 2.4 M, at least about 2.5 M, at least about 2.6 M, at least about 2.7 M, at least about 2.8 M, at least about 2.9 M, at least about 3 M, at least about 3.1 M, at least about 3.2 M, at least about 3.3 M, at least about 3.4 M, at least about 3.5 M, at least about 3.6 M, at least about 3.7 M, at least about 3.8 M, at least about 3.9 M, at least about 4 M, at least about 4.1 M, at least about 4.2 M, at least about 4.3 M, at least about 4.4 M, at least about 4.5 M, at least about 4.6 M, at least about 4.7 M, at least about 4.8 M, at least about 4.9 M, at least about 5 M, at least about 5.1 M, at least about 5.2 M, at least about 5.3 M, at least about 5.4 M, at least about 5.5 M, at least about 5.6 M, at least about 5.7 M, at least about 5.8 M, at least about 5.9 M, at least about 6 M, at least about 6.1 M, at least about 6.2 M, at least about 6.3 M, at least about 6.4 M, at least about 6.5 M, at least about 6.6 M, at least about 6.7 M, at least about 6.8 M, at least about 6.9 M, at least about 7 M, at least about 7.1 M, at least about 7.2 M, at least about 7.3 M, at least about 7.4 M, at least about 7.5 M, at least about 7.6 M, at least about 7.7 M, at least about 7.8 M, at least about 7.9 M, at least about 8 M, at least about 8.1 M, at least about 8.2 M, at least about 8.3 M, at least about 8.4 M, at least about 8.5 M, at least about 8.6 M, at least about 8.7 M, at least about 8.8 M, at least about 8.9 M, at least about 9 M, at least about 9.1 M, at least about 9.2 M, at least about 9.3 M, at least about 9.4 M, at least about 9.5 M, at least about 9.6 M, at least about 9.7 M, at least about 9.8 M, at least about 9.9 M, or at least about 10 M sodium hydroxide. In embodiments, the condition known to destabilize, unfold, degrade, or misfold a first polypeptide is exposure to 0.1 M sodium hydroxide. In embodiments, the condition known to destabilize, unfold, degrade, or misfold a first polypeptide is exposure to 0.1 M sodium hydroxide for about 30 minutes. In embodiments, the condition known to destabilize, unfold, degrade, or misfold a first polypeptide is exposure to 0.1 M sodium hydroxide for at least about 30 minutes. In embodiments, the condition known to destabilize, unfold, degrade, or misfold a first polypeptide is exposure to 0.1 M sodium hydroxide for 10-30 minutes.

In embodiments, the condition known to destabilize, unfold, degrade, or misfold a first polypeptide is a freeze-thaw cycle. In embodiments, a freeze-thaw cycle comprises freezing the solution containing the first polypeptide and then bringing the solution to a temperature above freezing. In embodiments, the first polypeptide is subjected to multiple freeze-thaw cycles. In embodiments, the first polypeptide is subjected to from 2 to 10, from 2 to 20, from 2 to 30, from 2 to 40, or from 2 to 50 freeze thaw cycles. In embodiments, the first polypeptide may be subjected to about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, or about 100 freeze-thaw cycles. In embodiments, the first polypeptide may be subjected to at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least about 31, at least about 32, at least about 33, at least about 34, at least about 35, at least about 36, at least about 37, at least about 38, at least about 39, at least about 40, at least about 41, at least about 42, at least about 43, at least about 44, at least about 45, at least about 46, at least about 47, at least about 48, at least about 49, at least about 50, at least about 51, at least about 52, at least about 53, at least about 54, at least about 55, at least about 56, at least about 57, at least about 58, at least about 59, at least about 60, at least about 61, at least about 62, at least about 63, at least about 64, at least about 65, at least about 66, at least about 67, at least about 68, at least about 69, at least about 70, at least about 71, at least about 72, at least about 73, at least about 74, at least about 75, at least about 76, at least about 77, at least about 78, at least about 79, at least about 80, at least about 81, at least about 82, at least about 83, at least about 84, at least about 85, at least about 86, at least about 87, at least about 88, at least about 89, at least about 90, at least about 91, at least about 92, at least about 93, at least about 94, at least about 95, at least about 96, at least about 97, at least about 98, at least about 99, or at least about 100 freeze-thaw cycles.

In embodiments, the condition known to destabilize, unfold, degrade, or misfold a first polypeptide is heat shock. In embodiments, the first polypeptide is heat shocked by heating the first polypeptide to at least 90° C., at least 91° C., at least 92° C., at least 93° C., at least 94° C., or at least 95° C. for about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, about 30 minutes, or more; placing a container containing the first polypeptide on ice, and then returning the fusion protein to room temperature. In embodiments, the first polypeptide is heat shocked by heating the first polypeptide to at least 90° C., at least 91° C., at least 92° C., at least 93° C., at least 94° C., or at least 95° C. for at least about 10 minutes, at least about 11 minutes, at least about 12 minutes, at least about 13 minutes, at least about 14 minutes, at least about 15 minutes, at least about 16 minutes, at least about 17 minutes, at least about 18 minutes, at least about 19 minutes, at least about 20 minutes, at least about 21 minutes, at least about 22 minutes, at least about 23 minutes, at least about 24 minutes, at least about 25 minutes, at least about 26 minutes, at least about 27 minutes, at least about 28 minutes, at least about 29 minutes, at least about 30 minutes, or more; placing a container containing the first polypeptide on ice, and then returning the fusion protein to room temperature.

In embodiments, heating to at least 90° C., at least 91° C., at least 92° C., at least 93° C., at least 94° C., or at least 95° C. is known to destabilize, unfold, degrade, or misfold a first polypeptide. In embodiments, the one or more conditions known to destabilize, unfold, degrade, or misfold a first polypeptide is heating to at least 90° C. In embodiments, the one or more conditions known to destabilize, unfold, degrade, or misfold a first polypeptide is heating to at least 95° C. In embodiments, the one or more conditions known to destabilize, unfold, degrade, or misfold a first polypeptide is heating to at least 90° C. for at least 30 minutes. In embodiments, the one or more conditions known to destabilize, unfold, degrade, or misfold a first polypeptide is heating to at least 95° C. for at least 30 minutes. In embodiments, the one or more conditions known to destabilize, unfold, degrade, or misfold a first polypeptide is heating to at least 95° C. for 10-30 minutes.

In embodiments, the condition known to destabilize, unfold, degrade, or misfold a first polypeptide is exposure to non-physiologic pH. The term non-physiologic pH refers to a pH that is not from 7.2 to about 7.4. In embodiments, the non-physiologic pH is acidic pH. In embodiments, the condition known to destabilize, unfold, degrade, or misfold a first polypeptide is exposure to an acidic pH. In embodiments, the condition known to destabilize, unfold, degrade, or misfold a first polypeptide is exposure to an acidic pH from about 0.5 to about 7, from about 0.5 to about 6, from about 0.5 to about 5, from about 0.5 to about 4, from about 0.5 to about 3, from about 0.5 to about 2, or from about 0.5 to about 1. In embodiments, the acidic pH is about 1, about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, about 3.1, about 3.2, about 3.3, about 3.4, about 3.5, about 3.6, about 3.7, about 3.8, about 3.9, about 4, about 4.1, about 4.2, about 4.3, about 4.4, about 4.5, about 4.6, about 4.7, about 4.8, about 4.9, about 5, about 5.1, about 5.2, about 5.3, about 5.4, about 5.5, about 5.6, about 5.7, about 5.8, about 5.9, about 6, about 6.1, about 6.2, about 6.3, about 6.4, about 6.5, about 6.6, about 6.7, about 6.8, about 6.9, about 7, or about 7.1, including all subranges and ranges therebetween. In embodiments, the condition known to destabilize, unfold, degrade, or misfold a first polypeptide is exposure to pH of about 4. In embodiments, the condition known to destabilize, unfold, degrade, or misfold a first polypeptide is exposure to pH of about 4 for about 10-30 minutes. In embodiments, the condition known to destabilize, unfold, degrade, or misfold a first polypeptide is exposure to pH of about 4 for about 30 minutes.

In embodiments, the condition known to destabilize, unfold, degrade, or misfold a first polypeptide is exposure to pH of about 4 and a temperature of to at least 95° C. for at least 30 minutes. In embodiments, the condition known to destabilize, unfold, degrade, or misfold a first polypeptide is exposure to pH of about 4 and a temperature of to at least 95° C. for 10-30 minutes.

In embodiments, the nonphysiologic pH is basic pH. In embodiments, the basic pH from about 7.5 to about 14, from about 7.5 to about 13, from about 7.5 to about 12, from about 7.5 to about 11, from about 7.5 to about 10, from about 7.5 to about 9, or from about 7.5 to about 8.5. In embodiments, the basic pH is about 8, about 8.1, about 8.2, about 8.3, about 8.4, about 8.5, about 8.6, about 8.7, about 8.8, about 8.9, about 9, about 9.1, about 9.2, about 9.3, about 9.4, about 9.5, about 9.6, about 9.7, about 9.8, about 9.9, about 10, about 10.1, about 10.2, about 10.3, about 10.4, about 10.5, about 10.6, about 10.7, about 10.8, about 10.9, about 11, about 11.1, about 11.2, about 11.3, about 11.4, about 11.5, about 11.6, about 11.7, about 11.8, about 11.9, about 12, about 12.1, about 12.2, about 12.3, about 12.4, about 12.5, about 12.6, about 12.7, about 12.8, about 12.9, about 13, about 13.1, about 13.2, about 13.3, about 13.4, about 13.5, about 13.6, about 13.7, about 13.8, about 13.9, or about 14, including all subranges and ranges therebetween.

In some embodiments, less than about 35%, less than about 30%, less than about 25%, less than about 20%, less than about 15%, less than about 10%, less than about 5%, less than about 3%, less than about 2%, or less than 1% of the activity of the first polypeptide is lost after exposure to one or more of the conditions as compared to a control polypeptide. In some embodiments, less than about 25% of the activity of the first polypeptide is lost after exposure to one or more of the conditions as compared to a control polypeptide. In some embodiments, less than about 20% of the activity of the first polypeptide is lost after exposure to one or more of the conditions as compared to a control polypeptide.

In some embodiments, a first polypeptide expressed as a fusion protein comprising a first polypeptide and a second polypeptide with phase behavior retains from about 50% to about 100%, from about 55% to about 100%, from about 60% to about 100%, from about 65% to about 100%, from about 70% to about 100%, from about 75% to about 100%, from about 80% to about 100%, from about 85% to about 100%, from about 90% to about 100%, or from about 95% to about 100% of its activity after exposure to one or more of the conditions as compared to a control polypeptide. In some embodiments, a first polypeptide retains at least 65%, at least 70%, at least 75%, at least 80%, at least 85%, at least 90%, at least 95%, at least 96%, at least 97%, at least 98%, at least 99%, or 100% of its activity after exposure to one or more of the conditions as compared to a control. In some embodiments, a first polypeptide retains at least 80% of its activity after exposure to one or more of the conditions as compared to a control. Activity of a first polypeptide may be measured using a functional assay, enzyme-linked immunosorbent assay, or flow cytometry.

In some embodiments, the first polypeptide retains its activity after exposure to temperatures ranging from −20° C. to about 35° C. (e.g., at about −20° C., about −19° C., about −18° C., about −17° C., about −16° C., about −15° C., about −14° C., about −13° C., about −12° C., about −11° C., about −10° C., about −9° C., about −8° C., about −7° C., about −6° C., about −5° C., about −4° C., about-3° C., about −2° C., about −1° C., about 0° C., about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C. including any values or subranges therebetween) for about 1 week to about 10 years (e.g., about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 1 month, about 2 months, about 3 months, about 4 months, about 5 months, about 6 months, about 7 months, about 8 months, about 9 months, about 10 months, about 11 months, about 12 months, about 1 year, about 2 years, about 3 years, about 4 years, about 5 years, about 6 years, about 7 years, about 8 years, about 9 years, or about 10 years, including all subranges and values therebetween). In some embodiments, the first polypeptide retains its activity after exposure to temperatures ranging from −20° C. to about 35° C. (e.g., at about −20° C., about −19° C., about −18° C., about −17° C., about −16° C., about −15° C., about −14° C., about −13° C., about −12° C., about −11° C., about −10° C., about −9° C., about −8° C., about −7° C., about −6° C., about −5° C., about −4° C., about −3° C., about −2° C., about −1° C., about 0° C., about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C. including any values or subranges therebetween) for about 1 week to about 10 years (e.g., at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 1 year, at least about 2 years, at least about 3 years, at least about 4 years, at least about 5 years, at least about 6 years, at least about 7 years, at least about 8 years, at least about 9 years, or at least about 10 years, including all subranges and values therebetween).

In some embodiments, the first polypeptide retains its activity after exposure to about 4° C. for about 1 week to about 10 years. In some embodiments, the first polypeptide retains its activity at about 4° C. for about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 1 month, about 2 months, about 3 months, about 4 months, about 5 months, about 6 months, about 7 months, about 8 months, about 9 months, about 10 months, about 11 months, about 12 months, about 1 year, about 2 years, about 3 years, about 4 years, about 5 years, about 6 years, about 7 years, about 8 years, about 9 years, about 10 years, or more, including all subranges and values therebetween. In some embodiments, the first polypeptide retains its activity at at least about 4° C. for at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 1 year, at least about 2 years, at least about 3 years, at least about 4 years, at least about 5 years, at least about 6 years, at least about 7 years, at least about 8 years, at least about 9 years, at least about 10 years, or more, including all subranges and values therebetween.

In some embodiments, the first polypeptide retains its activity after exposure to −20° C. for about 6 months to about 10 years. In some embodiments, the first polypeptide retains its activity at about 4° C. for about 6 months, about 7 months, about 8 months, about 9 months, about 10 months, about 11 months, about 12 months, about 1 year, about 2 years, about 3 years, about 4 years, about 5 years, about 6 years, about 7 years, about 8 years, about 9 years, about 10 years, or more, including all subranges and values therebetween. In some embodiments, the first polypeptide retains its activity at at least about 4° C. for at least about 6 months, at least about 7 months, at least about 8 months, at least about 9 months, at least about 10 months, at least about 11 months, at least about 12 months, at least about 1 year, at least about 2 years, at least about 3 years, at least about 4 years, at least about 5 years, at least about 6 years, at least about 7 years, at least about 8 years, at least about 9 years, at least about 10 years, or more, including all subranges and values therebetween.

In some embodiments, one or more of the following techniques is used to evaluate the ability of the methods of the disclosure to stabilize a first polypeptide: size exclusion chromatography, ion exchange and reversed phase high-performance liquid chromatography, sodium dodecyl sulfate polyacrylamide gel electrophoresis, capillary electrophoresis, potency assays, dynamic light scattering, spectroscopy, microscopy, and physicochemical measurements of appearance, pH, and particle size.

In some embodiments, expression of a first polypeptide as a fusion protein comprising a first polypeptide and a second polypeptide with phase behavior protects the first polypeptide from degrading during storage (e.g., freezing). In some embodiments, the methods described herein protect the first polypeptide from degrading, particularly during multiple freeze-thaw cycles. Aggregation of first polypeptide may be observed visually by microscopy and/or by a technique selected from the group consisting of x-ray scattering, laser diffraction, analytical ultracentrifugation, dynamic light scattering, nanoparticle tracking analysis, resonant mass measurement, size exclusion chromatography, gel permeation chromatography, light obscuration, and combinations thereof. In some embodiments, the first polypeptide may be frozen and stored as a fusion protein comprising a first polypeptide and a second polypeptide with phase behavior at temperatures from about −80° C. to about 40° C., for example, about −80° C., about −75° C., about −70° C., about −65° C., about −60° C., about −55° C., about −50° C., about −45° C., about −40° C., about −35° C., about −30° C., about −25° C., about −20° C., about −15° C., about −10° C., about −5° C., about 0° C., about 4° C., about 5° C., about 10° C., about 15° C., about 20° C., about 25° C., about 30° C., about 35° C., or about 40° C.

In some embodiments, when a first polypeptide is stored as a fusion protein comprising a first polypeptide and a second polypeptide with phase behavior, the shelf life of the first polypeptide is at least about 10% longer as compared to a first polypeptide that is not expressed as a fusion protein comprising a second polypeptide with phase behavior. For example, in some embodiments, the shelf life is at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 450%, or at least about 500% longer than the shelf life of a first polypeptide that is not expressed as a fusion protein comprising a second polypeptide with phase behavior stored at the same temperature. In some embodiments, when a first polypeptide is stored as a fusion protein comprising a first polypeptide and a second polypeptide with phase behavior, the shelf life of the first polypeptide is at least 1 month longer as compared to a first polypeptide that is not expressed as a fusion protein comprising a second polypeptide with phase behavior. For example, in some embodiments, the shelf life is at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months longer than the shelf life of a first polypeptide that is not expressed as a fusion protein comprising a second polypeptide with phase behavior stored at the same temperature.

In some embodiments, a fusion polypeptide comprising a first polypeptide and a second polypeptide with phase behavior is stored at about −80° C. In some embodiments, a fusion polypeptide comprising a first polypeptide and a second polypeptide with phase behavior is stored at about −20° C. In some embodiments, a fusion polypeptide comprising a first polypeptide and a second polypeptide with phase behavior is stored at about 4° C. In some embodiments, a fusion polypeptide comprising a first polypeptide and a second polypeptide with phase behavior is stored at about 37° C. In some embodiments, when a fusion polypeptide comprising a first polypeptide and a second polypeptide with phase behavior is stored at about −80° C., about −20° C., about 4° C., or about 37° C., the shelf life of the first polypeptide is at least about 10% longer than if it was not expressed as a fusion protein and stored under the same conditions. For example, the shelf life of the first polypeptide may be at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 450%, or at least about 500% longer than the shelf life of the first polypeptide not expressed as a fusion protein comprising a first polypeptide and a second polypeptide with phase behavior at the same temperature. As used herein, increased shelf life may refer to an increase in the amount of time a first polypeptide is stored and still retains its function. For example, a first polypeptide comprising a monoclonal antibody that binds to programmed cell death protein 1 (PD1) that retains its function retains the same affinity for PD1.

### Methods for Improving Production of a First Polypeptide

In some embodiments, provided herein is a method for improving the yield of a first polypeptide, the method comprising: (i) expressing a fusion protein comprising the first polypeptide and a second polypeptide having phase behavior; and (ii) separating the first polypeptide from the second polypeptide wherein the yield of the first polypeptide is improved when expressed as the fusion protein compared to a yield of the first polypeptide when the first polypeptide is not expressed as a fusion protein.

In some embodiments, the first polypeptide is separated from the second polypeptide by protease cleavage. In some embodiments, the protease is furin.

In some embodiments, the fusion protein is expressed in a host cell, selected from a mammalian cell, a bacterial cell, a fungal cell, a yeast cell, and a plant cell. In some embodiments, the host cell is E. coli. In some embodiments, the host cell is a CHO cell. In some embodiments, the host cell is a HEK293 cell.

In some embodiments, provided herein is a method of increasing yield of the first polypeptide during production thereof. In some embodiments, the yield of the first polypeptide produced as a fusion protein comprising a second polypeptide with phase behavior is higher than the yield of a first polypeptide expressed not as a fusion protein (i.e., without being fused to a second polypeptide with phase behavior). In embodiments, the yield of the first polypeptide when it is expressed as a fusion protein comprising a second polypeptide with phase behavior is about 1 mg per liter to about 1000 mg per liter. In embodiments, the yield of the first polypeptide when it is expressed as a fusion protein comprising a second polypeptide with phase behavior is about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, or about 1000 mg per liter of host cell suspension. In embodiments, the yield of the first polypeptide when it is expressed as a fusion protein comprising a second polypeptide with phase behavior is at least about 1 mg, at least about 2 mg, at least about 3 mg, at least about 4 mg, at least about 5 mg, at least about 6 mg, at least about 7 mg, at least about 8 mg, at least about 9 mg, at least about 10 mg, at least about 11 mg, at least about 12 mg, at least about 13 mg, at least about 14 mg, at least about 15 mg, at least about 16 mg, at least about 17 mg, at least about 18 mg, at least about 19 mg, at least about 20 mg, at least about 21 mg, at least about 22 mg, at least about 23 mg, at least about 24 mg, at least about 25 mg, at least about 26 mg, at least about 27 mg, at least about 28 mg, at least about 29 mg, at least about 30 mg, at least about 31 mg, at least about 32 mg, at least about 33 mg, at least about 34 mg, at least about 35 mg, at least about 36 mg, at least about 37 mg, at least about 38 mg, at least about 39 mg, at least about 40 mg, at least about 41 mg, at least about 42 mg, at least about 43 mg, at least about 44 mg, at least about 45 mg, at least about 46 mg, at least about 47 mg, at least about 48 mg, at least about 49 mg, at least about 50 mg, at least about 51 mg, at least about 52 mg, at least about 53 mg, at least about 54 mg, at least about 55 mg, at least about 56 mg, at least about 57 mg, at least about 58 mg, at least about 59 mg, at least about 60 mg, at least about 61 mg, at least about 62 mg, at least about 63 mg, at least about 64 mg, at least about 65 mg, at least about 66 mg, at least about 67 mg, at least about 68 mg, at least about 69 mg, at least about 70 mg, at least about 71 mg, at least about 72 mg, at least about 73 mg, at least about 74 mg, at least about 75 mg, at least about 76 mg, at least about 77 mg, at least about 78 mg, at least about 79 mg, at least about 80 mg, at least about 81 mg, at least about 82 mg, at least about 83 mg, at least about 84 mg, at least about 85 mg, at least about 86 mg, at least about 87 mg, at least about 88 mg, at least about 89 mg, at least about 90 mg, at least about 91 mg, at least about 92 mg, at least about 93 mg, at least about 94 mg, at least about 95 mg, at least about 96 mg, at least about 97 mg, at least about 98 mg, at least about 99 mg, at least about 100 mg, at least about 110 mg, at least about 120 mg, at least about 130 mg, at least about 140 mg, at least about 150 mg, at least about 160 mg, at least about 170 mg, at least about 180 mg, at least about 190 mg, at least about 200 mg, at least about 210 mg, at least about 220 mg, at least about 230 mg, at least about 240 mg, at least about 250 mg, at least about 260 mg, at least about 270 mg, at least about 280 mg, at least about 290 mg, at least about 300 mg, at least about 310 mg, at least about 320 mg, at least about 330 mg, at least about 340 mg, at least about 350 mg, at least about 360 mg, at least about 370 mg, at least about 380 mg, at least about 390 mg, at least about 400 mg, at least about 410 mg, at least about 420 mg, at least about 430 mg, at least about 440 mg, at least about 450 mg, at least about 460 mg, at least about 470 mg, at least about 480 mg, at least about 490 mg, at least about 500 mg, at least about 510 mg, at least about 520 mg, at least about 530 mg, at least about 540 mg, at least about 550 mg, at least about 560 mg, at least about 570 mg, at least about 580 mg, at least about 590 mg, at least about 600 mg, at least about 610 mg, at least about 620 mg, at least about 630 mg, at least about 640 mg, at least about 650 mg, at least about 660 mg, at least about 670 mg, at least about 680 mg, at least about 690 mg, at least about 700 mg, at least about 710 mg, at least about 720 mg, at least about 730 mg, at least about 740 mg, at least about 750 mg, at least about 760 mg, at least about 770 mg, at least about 780 mg, at least about 790 mg, at least about 800 mg, at least about 810 mg, at least about 820 mg, at least about 830 mg, at least about 840 mg, at least about 850 mg, at least about 860 mg, at least about 870 mg, at least about 880 mg, at least about 890 mg, at least about 900 mg, at least about 910 mg, at least about 920 mg, at least about 930 mg, at least about 940 mg, at least about 950 mg, at least about 960 mg, at least about 970 mg, at least about 980 mg, at least about 990 mg, or at least about 1000 mg per liter of host cell suspension. In some embodiments, the yield of the first polypeptide is greater than 15 mg per liter, greater than 30 mg per liter, greater than 50 mg per liter, greater than 100 mg per liter, greater than 200 mg per liter, or greater than 200 mg per liter of host cell suspension. In embodiments, the yield of the first polypeptide is greater than 75 mg per liter of host cell suspension. Cell suspension may be referred to interchangeably with cell culture.

In some embodiments, the yield of the first polypeptide produced as a fusion protein comprising a second polypeptide with phase behavior is at least 2-fold, at least 3-fold, at least 4-fold, at least 5-fold, at least 6-fold, at least 7-fold, at least 8-fold, at least 9-fold, at least 10-fold, at least 50-fold, at least 100-fold, or more, than the yield of the first polypeptide not expressed as a fusion protein (i.e., expressed without being fused to a second polypeptide with phase behavior). In embodiments, the yield of the first polypeptide produced as a fusion protein comprising a second polypeptide with phase behavior is about 3-fold greater than the yield of the first polypeptide not expressed as a fusion protein.

In some embodiments, the yield of the first polypeptide produced as a fusion protein comprising a second polypeptide with phase behavior is at least about 50%, at least about 75%, at least about 100%, at least about 125%, at least about 150%, at least about 175%, at least about 200%, at least about 225%, at least about 250%, at least about 275%, at least about 300%, at least about 325%, at least about 350%, at least about 375%, at least about 400%, at least about 425%, at least about 450%, at least about 475%, at least about 500%, at least about 525%, at least about 550%, at least about 575%, at least about 600%, at least about 625%, at least about 650%, at least about 675%, at least about 700%, at least about 725%, at least about 750%, at least about 775%, at least about 800%, at least about 825%, at least about 850%, at least about 875%, at least about 900%, at least about 925%, at least about 950%, at least about 975%, at least about 1000%, at least about 1100%, at least about 1125%, at least about 1150%, at least about 1175%, at least about 1200%, at least about 1225%, at least about 1250%, at least about 1275%, at least about 1300%, at least about 1325%, at least about 1350%, at least about 1375%, at least about 1400%, at least about 1425%, at least about 1450%, at least about 1475%, at least about 1500%, at least about 1525%, at least about 1550%, at least about 1575%, at least about 1600%, at least about 1625%, at least about 1650%, at least about 1675%, at least about 1700%, at least about 1725%, at least about 1750%, at least about 1775%, at least about 1800%, at least about 1825%, at least about 1850%, at least about 1875%, at least about 1900%, at least about 1925%, at least about 1950%, at least about 1975%, or at least about 2000% higher than the yield of a first polypeptide when not expressed as a fusion protein, including all ranges and values therebetween. In embodiments, the yield of the first polypeptide is about 300% higher than the yield of a first polypeptide that is not expressed as a fusion protein.

### Methods for Purifying a First Polypeptide of a Fusion Protein

Also provided herein is a method for purifying a first polypeptide, the method comprising: i) providing a fusion protein comprising the first polypeptide and a second polypeptide having phase behavior; ii) applying a first environmental factor to reversibly aggregate the fusion protein; iii) separating the fusion protein aggregates from at least one contaminant; and iv) applying a second environmental factor to disaggregate the fusion protein. In some embodiments, the fusion protein is any fusion protein described herein.

In some embodiments, the at least one contaminant is a solvent, a protein, a peptide, a carbohydrate, a nucleic acid, a virus, a cell (e.g., a bacterial, yeast, or mammalian cell), a carbohydrate, a lipid, or a lipopolysaccharide. In some embodiments, the contaminant is an endotoxin or a mycotoxin.

Without being bound by theory, the application of a first environmental factor causes aggregation of the fusion protein via intermolecular interactions between the polypeptides with phase behavior, resulting in fusion protein aggregates. In some embodiments, an environmental factor causes the formation of fusion protein aggregates that are larger in size that the size of the fusion protein before introduction of the environmental factor. As used herein, the phrase “increase in size” may refer to an increase in the diameter of the fusion protein or an increase in the mass of the fusion protein. In some embodiments, the increase in size is an increase in the molar mass of the fusion protein. In some embodiments, the increase in size is an increase in the hydrodynamic radius of the fusion protein.

In some embodiments, the size increase is stabilized by non-covalent interactions between polypeptides with phase behavior. In some embodiments, the non-covalent interactions are dipole-dipole forces, van der Waals forces, London Dispersion forces, hydrogen bonding, hydrophobic interactions, and/or electrostatic interactions.

In some embodiments, the size of the fusion protein aggregates is at least about 2-fold, at least about 5-fold, at least about 10-fold, at least about 15-fold, at least about 20-fold, at least about 25-fold, at least about 30-fold, at least about 35-fold, at least about 40-fold, at least about 45-fold, at least about 50-fold, at least about 55-fold, at least about 60-fold, at least about 65-fold, at least about 70-fold, at least about 75-fold, at least about 80-fold, at least about 85-fold, at least about 90-fold, at least about 95-fold, at least about 100-fold, greater than the size of the fusion protein before introduction of the environmental factor. In some embodiments, the size of the fusion protein aggregates is at least 2-fold larger than the size of the fusion protein before introduction of the environmental factor. In some embodiments, the size of the fusion protein aggregates is at least 5-fold larger than the size of the fusion protein before introduction of the environmental factor. In some embodiments, the size of the fusion protein aggregates is at least 10-fold larger than the size of the fusion protein before introduction of the environmental factor. In some embodiments, the size of the fusion protein aggregates is at least 25-fold larger than the size of the fusion protein before introduction of the environmental factor.

In some embodiments, the increased size of the fusion protein aggregates compared to the fusion protein can be observed visually with an unaided eye. For example, the increased size may cause a composition comprising the complex to change color, clarity, viscosity, and/or may cause the complex to change solubility (e.g., to precipitate from solution), wherein such change is observable by a human without the use of any special equipment.

In some embodiments, a person of skill in the art may measure the increased size of the fusion protein aggregates according to known methods in the art. In some embodiments, the increased size can be measured utilizing a technique selected from the group consisting of x-ray scattering, small angle x-ray scattering, wide angle x-ray scattering, dynamic light scattering, analytical ultracentrifugation, size exclusion chromatography, and photon correlation spectroscopy.

In some embodiments, the environmental factor is an environmental described herein. For example, in some embodiments, the environmental factor is (a) a change in one or more of temperature, pH, salt concentration or pressure; (b) the addition of one or more surfactants, cofactors, vitamins, molecular crowding agents, enzymes, denaturing agents; or (c) the application of electromagnetic waves.

In some embodiments, the fusion protein aggregates are separated from one or more impurities by washing the fusion protein aggregates. In some embodiments, washing the fusion protein aggregates does not interfere with aggregation of the fusion protein. In some embodiments, the fusion protein aggregates are washed with a buffer. Non-limiting examples of buffers include sodium acetate, saline, glycine-HCL, cacodylate buffer, Tris-HCl, 4-(2-hydroxyethyl)-1-piperazineethanesulfonic acid (HEPES), 2-(N-morpholino)ethanesulfonic acid (MES), 3-(N-morpholino)propanesulfonic acid (MOPS), citrate, phosphate buffer, tris(hydroxymethyl)methylamino]propanesulfonic acid (TAPS), and tris(hydroxymethyl)aminomethane (Tris). In some embodiments, the buffer comprises one or more of arginine, histidine, urea, pluronic acid, and triton-x-100. In some embodiments, the AAV-purification matrix complex is washed with a solvent. Non-limiting examples of solvents include acetone, acetonitrile, dimethylformamide, water, ethanol, toluene, methyl acetate, and ethyl acetate.

In some embodiments, the fusion protein aggregates are separated from at least one impurity on the basis of size. In some embodiments, the fusion protein aggregates are separated from at least one impurity on the basis of diameter. In some embodiments, the fusion protein aggregates are separated from at least one impurity on the basis of radius. In some embodiments, the fusion protein aggregates are separated from at least one impurity on the basis of mass. In some embodiments, the fusion protein aggregates are separated from at least one impurity on the basis of molar mass. In some embodiments, the AAV-purification matrix is fusion protein aggregates are separated from at least one impurity on the basis of size by using a technique selected from the group consisting of centrifugation, tangential flow filtration, analytical ultracentrifugation, membrane chromatography, high performance liquid chromatography, size exclusion chromatography, normal flow filtration, acoustic wave separation, centrifugation, counterflow centrifugation, and fast protein liquid chromatography.

In some embodiments, the separation is achieved using acoustic wave separation. In some embodiments, the acoustic waves have a frequency between about 1 Hz and 2000 kHz. In some embodiments, the acoustic waves have a frequency of about 1 Hz, about 5 Hz, about 10 Hz, about 20 Hz, about 30 Hz, about 40 Hz, about 50 Hz, about 60 Hz, about 70 Hz, about 80 Hz, about 90 Hz, about 100 Hz, about 200 Hz, about 300 Hz, about 400 Hz, about 500 Hz, about 600 Hz, about 700 Hz, about 800 Hz, about 900 Hz, about 1 kHz, about 100 kHz, about 200 kHz, about 300 kHz, about 400 kHz, about 500 kHz, about 600 kHz, about 700 kHz, about 800 kHz, about 900 kHz, about 1000 kHz, about 1100 kHz, about 1200 kHz, about 1300 kHz, about 1400 kHz, about 1500 kHz, about 1600 kHz, about 1700 kHz, about 1800 kHz, about 1900 kHz, or about 2000 kHz.

In some embodiments, the fusion protein aggregates are separated from at least one impurity on the basis of size using centrifugation. In some embodiments, between about 100 relative centrifugal force (RCF) and about 16,000 RCF, for example, about 500 to about 16,000 RCF, about 1,000 RCF to 16,000 RCF, are applied to separate the fusion protein aggregates from at least one impurity. In some embodiments, at least 500 relative centrifugal force (RCF) are applied to separate the fusion protein aggregates from at least one impurity, for example, at least about 500 RCF, at least about 600 RCF, at least about 700 RCF, at least about 800 RCF, at least about 900 RCF, at least about 1000 RCF, at least about 2000 RCF, at least about 3000 RCF, at least about 3500 RCF, at least about 4000 RCF, at least about 5000 RCF, at least about 6000 RCF, at least about 7000 RCF, at least about 8000 RCF, at least about 9000 RCF, at least about 10,000 RCF, at least about 11,000 RCF, at least about 12,000 RCF, at least about 13,000 RCF, at least about 14,000 RCF, at least about 15,000 RCF, at least about 16,000 RCF, at least about 17,000 RCF, at least about 18,000 RCF, at least about 19,000 RCF, or at least about 20,000 RCF.

In some embodiments, the fusion protein aggregates are separated from at least one impurity on the basis of size by using TFF. In some embodiments, TFF may be used to separate the fusion protein aggregates from at least one impurity on the basis of size, a process also referred to herein as “diafiltration.” Diafiltration comprises both washing and elution steps. Washing removes impurities contained in the composition comprising the fusion protein aggregates. Elution separates purified first polypeptide from the second polypeptide. In some embodiments, the fusion protein aggregates are concentrated using TFF. In some embodiments, TFF may be used to increase the concentration of fusion protein aggregates within a composition, a process also referred to herein as “concentration.”

Tangential flow filtration employs both microfiltration and ultrafiltration membranes to separate and/or concentrate molecules. Microfiltration membranes typically have pore sizes between 0.1 μm and 10 μm. Ultrafiltration membranes typically have smaller pore sizes than microfiltration membranes with pore sizes between 0.001 μm and 0.1 μm. In some embodiments, a membrane with a pore size between about 0.001 μm and about 10 μm is utilized in the methods of the disclosure. In some embodiments, the membrane has a pore size of about 0.001 μm, about 0.01 μm, about 0.05 μm, about 0.1 μm, about 0.2 μm, about 0.3 μm, about 0.4 μm, about 0.5 μm, about 0.6 μm, about 0.7 μm, about 0.8 μm, about 0.9 μm, about 1.0 μm, about 2 μm, about 3 μm, about 4 μm, about 5 μm, about 6 μm, about 7 μm, about 8 μm, about 9 μm, or about 10 μm, including all values and ranges in between thereof. In some embodiments, the membrane has a pore size of about 0.1 μm. In some embodiments, the membrane has a pore size of about 0.2 μm.

In some embodiments, the membrane is made of hydrophilized poly(vinylildene difluoride) (PVDF), polyetheresulfone (PES), cellulose phosphate, diethylaminoethyl cellulose, polysufone, regenerated cellulose, nylon, cellulose nitrate, cellulose acetate, pegylated PES, and sulfonated PES.

In TFF, a membrane is placed tangentially to the flow of a fluid mixture to cause the fluid mixture to flow tangentially over a first side of the membrane. At the same time, a fluid media is placed in contact with a second surface of the membrane. A transmembrane pressure is the force that drives fluid through the membrane, carrying along permeable molecules.

In some embodiments, separation of fusion protein aggregates from the one or more contaminants or impurities on the basis of size is performed using TFF with a transmembrane pressure of between about 0.1 bar to about 3 bar. In some embodiments, the transmembrane pressure is about 0.1 bar, about 0.2 bar, about 0.3 bar, about 0.4 bar, about 0.5 bar, about 0.6 bar, about 0.7 bar, about 0.8 bar, about 0.9 bar, about 1.0 bar, about 1.1 bar, about 1.2 bar, about 1.3 bar, about 1.4 bar, about 1.5 bar, about 1.6 bar, about 1.7 bar, about 1.8 bar, about 1.9 bar, about 2.0 bar, about 2.1 bar, about 2.2 bar, about 2.3 bar, about 2.4 bar, about 2.5 bar, about 2.6 bar, about 2.7 bar, about 2.8 bar, about 2.9 bar, or about 3.0 bar, including all values and ranges in between thereof. In some embodiments, the transmembrane pressure is about 1.5 bar.

In some embodiments, the cross flow rate is tuned to improve the separation of the fusion protein aggregates described herein from the one or more contaminants. The cross flow rate is the rate of solution flow through the feed channel and across the membrane. It provides the force that sweeps away molecules that can restrict filtrate flow. In some embodiments, the cross flow rate is between about 500 L/m2/h and about 2000 L/m2/h. In some embodiments, the cross flow rate is between about 500 L/m2/h, about 600 L/m2/h, about 700 L/m2/h, about 800 L/m2/h, about 900 L/m2/h, about 1000 L/m2/h, about 1100 L/m2/h, about 1200 L/m2/h, about 1300 L/m2/h, about 1400 L/m2/h, about 1500 L/m2/h, about 1600 L/m2/h, about 1700 L/m2/h, about 1800 L/m2/h, about 1900 L/m2/h, or about 2000 L/m2/h, including all values and ranges in between thereof. In some embodiments, the cross flow rate is about 960 L/m2/h. In some embodiments, TFF separation occurs by using a membrane that retains the fusion protein aggregates while passing the contaminant.

In some embodiments, after separation of the fusion protein aggregates from the at least one contaminant, the first polypeptide is separated from the second polypeptide. In some embodiments, the first polypeptide is separated from the second polypeptide by introducing an enzyme that cleaves an amide bond separating the first polypeptide from the second polypeptide. In some embodiments, the enzyme is a protease.

In some embodiments, after separation of the fusion protein aggregates are separated from the at least one contaminant, an environmental factor is applied to disaggregate the fusion protein aggregates. The environmental factor applied to disaggregate the fusion protein aggregates may be any of the environmental factors described herein.

In some embodiments, after separation of the fusion protein aggregates from at least one impurity, the first polypeptide is eluted as a fusion protein comprising a first polypeptide and a second polypeptide with phase behavior. In some embodiments, the first polypeptide is eluted as a disaggregated fusion protein comprising the first polypeptide and a second polypeptide with phase behavior.

In some embodiments, the environmental factor comprises changing the pH of the composition comprising the first polypeptide. In some embodiments, the pH is increased by about 0.1, about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, or about 6.0 units. In some embodiments, the pH is decreased by about 0.1, about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, or about 6.0 units. In some embodiments, the purified fusion protein aggregates at a pH of about 2. In some embodiments, the fusion protein is purified at a pH of about 3.

In some embodiments, the environmental factor comprises changing the temperature of the composition comprising the fusion protein. In some embodiments, the temperature is increased 0.5° C., about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., or about 40° C. In some embodiments, the temperature is decreased about 0.5° C., about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., or about 40° C.

In some embodiments, the environmental factor comprises changing the ionic strength of the composition comprising the fusion protein. In some embodiments, the change in ionic strength is brought about by increasing the concentration of salt. In some embodiments, the change in ionic strength is brought about by decreasing the concentration of salt. Non-limiting examples of salts include sodium chloride, potassium chloride, ammonium chloride, sodium acetate, sodium citrate, glycine, arginine, copper sulfate, sodium iodide, ammonium sulfate, and sodium sulfate. In some embodiments, a dialysis is used to change the concentration of salt in the composition comprising the fusion protein, contaminant, and/or molecule.

In some embodiments, the environmental factor comprises addition of a reducing agent to the composition comprising the fusion protein. In some embodiments, the one or more reducing agents is selected from the group consisting of dithiothreitol (DTT), 2-mercaptoethanol (BME), Tris (2-carboxyethyl) phosphine (TCEP), hydrazine, boron hydrides, amine boranes, lower alkyl substituted amine boranes, triethanolamine, and N,N,N′,N′-tetramethylethylenediamine (TEMED).

In some embodiments, the method of purifying a fusion protein comprising a first polypeptide described herein is completed in about 30 minutes to about 24 hours. In some embodiments, the methods described herein are completed in about 30 minutes to about 24 hours. In some embodiments, the methods are completed in about 30 minutes, about 1 hr, about 2 hr, about 3 hr, about 4 hr, about 5 hr, about 6 hr, about 7 hr, about 8 hr, about 9 hr, about 10 hr, about 11 hr, about 12 hr, about 13 hr, about 14 hr, about 15 hr, about 16 hr, about 17 hr, about 18 hr, about 19 hr, about 20 hr, about 21 hr, about 22 hr, about 23 hr, or about 24 hr. In some embodiments, the method of purifying a fusion protein and/or first polypeptide described herein is completed in about 2 hours to about 10 hours.

In some embodiments, after purification of the fusion protein, the first polypeptide is separated from the second polypeptide. In some embodiments, the first polypeptide is separated from the second polypeptide by introducing an enzyme that cleaves an amide bond separating the first polypeptide from the second polypeptide. In some embodiments, the enzyme is a protease.

In some embodiments, the purification yield of the fusion protein and/or the first polypeptide is at least 70%, at least 80%, at least 90%, at least 95%, at least 96%, at least 97%, at least 98%, or at least 99%.

In some embodiments, the fusion protein and/or the first polypeptide is purified to at least 70%, at least 80%, at least 90%, at least 95%, at least 96%, at least 97%, at least 98%, or at least 99% purity.

In some embodiments, the methods described herein enable the purification of at least 0.1 kg, at least about 0.2 kg, at least about 0.3 kg, at least about 0.4 kg, at least about 0.5 kg, at least about 0.6 kg, at least about 0.7 kg, at least about 0.8 kg, at least about 0.9 kg, at least about 1 kg, at least about 2 kg, at least about 3 kg, at least about 4 kg, at least about 5 kg, at least about 6 kg, at least about 7 kg, at least about 8 kg, at least about 9 kg, at least about 10 kg, or more of fusion protein and/or the first polypeptide per day, including all values and ranges in between thereof.

### Methods for Performing an Enzymatic Process on a Nucleic Acid Substrate

The fusion proteins may be used to perform enzymatic processes on a nucleic acid substrate, in a controlled fashion. In some embodiments, the first protein of the fusion protein is an enzyme, or a catalytic fragment or derivative thereof. For example, the process may be performed in a two-phase composition. In such a composition, the substrate may be present in a first phase, and the fusion protein may be present in a second phase. When desired, an environmental factor may be applied, which causes the fusion protein to enter the first phase, thereby coming into contact with a substrate. In some embodiments, a second environmental factor may be applied, which causes the fusion protein to leave the first phase, so that it may no longer contact the substrate. This process may be used to perform multi-step enzymatic processes on a substrate, in a controlled fashion. For example, two-phase composition may be provided wherein the substrate is present in a first phase, and a plurality of fusion proteins may be present in the second phase. The plurality of fusion proteins comprise fusion proteins comprising different phase behaviors. When desired, a first environmental factor may be applied, which causes one or more fusion proteins to contact and/or be removed from contacting the substrate. Additionally, a second environmental factor, which causes one or more additional fusion proteins to contact and/or be removed from the substrate. Such processes may be useful for synthesis of biologics of interest, such as in vitro transcription of RNA.

In embodiments, the nucleic acid substrate is DNA or RNA. In embodiments, the DNA or RNA is single stranded. In embodiments, the DNA or RNA is double stranded. In embodiments, the nucleic acid is RNA, and the RNA is selected from messenger RNA (mRNA), transfer RNA (tRNA), microRNA, or ribosomal RNA (rRNA). In embodiments, the mRNA is a small RNA. Small RNA comprise from about 18 to about 30 nucleotides, for example, about 18, 19, 20, 21, 22, 23, 24, 25, 26, 27, 28, 29, or 30 nucleotides.

In embodiments, the enzyme is any one of the NBP described herein. In embodiments, the NBP is selected from a T7 RNA polymerase, Rnase inhibitor, 2′-O-Methyltransferase, Inorganic Pyrophosphatase, Poly(A) Polymerase, DNase I, Calf intestinal phosphatase, Antarctic phosphatase, D1 subunit of the Vaccinia virus mRNA capping enzyme, Guanine-7-methyltransferase (found in D1 subunit of the Vaccinia virus mRNA capping enzyme), Guanylyltransferase (found in D1 subunit of the Vaccinia virus mRNA capping enzyme), RNA triphosphatase (found in D1 subunit of the Vaccinia virus mRNA capping enzyme), and D12 subunit of vaccinia virus mRNA capping enzyme, a stem-loop binding protein, a heterogenous ribonucleoprotein (hnRNP), GroEL, Edc3, DHX9, Xrn1, Dcp1, Dcp2, LAF-1, MEG-1, MEG-3, ASF/SF2 splicing factor, serine/arginine rich splicing factor 4 (SRp75), the serine and arginine rich splicing factor 1 (SRSF1), the L3 ribosomal protein, the L4 ribosomal protein, the L13 ribosomal protein, the L20 ribosomal protein, the L22 ribosomal protein, the L24 ribosomal protein, the L24e ribosomal protein, the S12 ribosomal protein, the S14 ribosomal protein, and the eukaryotic initiation factor 4E-binding protein 1 (4EBP1), Tat, Rev, RSG-1.2 peptide, poly(A)-binding protein (PABP), eukaryotic translation initiation factor 4E (eIF4E), eukaryotic translation initiation factor3 Subunit D (eIF3D), heterogenous nuclear ribonucleoproteins (hnRNPs), RNA-specific adenosine deaminase 1 (ADAR1), RNA-specific adenosine deaminase 2 (ADAR2), CspB from Bacillus subtilis (Bscscp), Y-box protein 1 cold shock domain (YB1-CSD), a Fox-1 protein (FOX1), poly(A)-binding protein (PABP), Staufen protein, TIS11d, zinc finger protein (ZNF), Z-DNA binding protein 1 (ZBP1), retinoic acid-inducible gene-I (RIG-I) like protein, toll like receptor 7 (TLR7), toll like receptor 8 (TLR3), toll like receptor 8 (TLR8), retinoic acid-inducible gene I (RIG-I), melanoma differentiation-associated protein 5 (MDA5), interferon induced protein with tetratricopeptide repeats 1 (IFIT1), protein kinase R (PKR), an oligoadenylate synthase-like (OASL) protein (e.g., OAS1, OAS2, OAS3, or OASL), ribonuclease E (RNASE E), gamma-interferon-inducible protein Ifi-16 (IF116), cyclic GMP-AMP synthase (cGAS), or a catalytic fragment thereof.

In embodiments, the NBP comprises one or more of the following domains: a short linear motif (SLIM), an RG[G] repeat, an RGG repeat, a RS/RG rich domain, a K/R basic patch, a molecular recognition feature, a low complexity sequence, an RNA recognition motif, a double-stranded RNA binding domain, a K homology domain, a zinc finger domain (e.g., CCHH ZF domain, a CCCC (Ran-BP2) domain, a CCCH ZF domain), an RGG domain, a Pumillo family domain, a pentatricopeptide domain, a cold shock domain, a helicase domain, a La motif, a Piwi-Argonaute-Zwille (PAZ) domain, a P-element induced wimpy testis, a pseudouridine synthase and archaeosine transglycosylate (PUA), a Pumillo-like repeat (PUM), a ribosomal S1-like (S1), Sm and Like-Sm (Sm/Lsm) repeat, thiouridine synthases and RNA methylases and pseudouridine synthases (THUMP), or a domain with YT521-B homology.

In embodiments, the polypeptide with phase behavior is any polypeptide with phase behavior described herein.

In some embodiments, a method for performing an enzymatic process on a substrate, comprises i) providing a first fusion protein comprising a first enzyme and a polypeptide having a first phase behavior; and ii) applying a first environmental factor, which allows the first enzyme to contact the substrate. In some embodiments, the method further comprises an additional environmental factor, which separates the first enzyme from the substrate.

In some embodiments, a method for performing a multi-step enzymatic process on a substrate, comprises i) providing a first fusion protein comprising a first enzyme and a polypeptide having a first phase behavior; ii) providing a second fusion protein comprising a second enzyme and polypeptide having a second phase behavior; iii) applying a first environmental factor, which allows the first enzyme to contact the substrate; and iv) applying a second environmental factor, which allows the second enzyme to contact the substrate. In some embodiments, the method further comprises v) applying a third environmental factor, which separates the first enzyme from the substrate. In some embodiments, the method further comprises and vi) applying a fourth environmental factor, which separates the second enzyme from the substrate.

In some embodiments, the method further comprises providing additional fusion proteins, for example, a third fusion protein, a fourth fusion protein, a fifth fusion protein, and so on. Each additional fusion protein comprises an additional enzyme and polypeptide with phase behavior. For example, a third fusion protein comprises a third enzyme and a polypeptide having a third phase behavior. In some embodiments, a plurality of fusion proteins is provided wherein each fusion protein comprises a different enzyme, but at least two of the fusion proteins comprise a polypeptide with the same phase behavior.

In some embodiments, provided herein is a method for performing a multi-step enzymatic process on a substrate, comprising: i) providing a first fusion protein comprising a first enzyme and a polypeptide having a first phase behavior; ii) providing a second fusion protein comprising a second enzyme and polypeptide having a second phase behavior; iii) applying a first environmental factor, which allows the first enzyme to contact, isolate, and/or concentrate the substrate; and iv) applying a second environmental factor, which allows the second enzyme to contact, isolate, and/or concentrate the substrate. In some embodiments, provided herein is a method for contacting, isolating, and/or purifying a substrate, the method comprising: i) providing a first fusion protein comprising a first enzyme and a polypeptide having a first phase behavior; ii) providing a second fusion protein comprising a second enzyme and polypeptide having a second phase behavior; iii) applying a first environmental factor, which allows the first enzyme to contact, isolate, and/or concentrate the substrate; and iv) applying a second environmental factor, which allows the second enzyme to contact, isolate, and/or concentrate the substrate. In some embodiments, the methods described herein allow the formation of concentrated droplets comprising the fusion protein and substrate. For example, in some embodiments, addition of a salt to a composition comprising (i) a fusion protein comprising the enzyme polyadenylate polymerase and a polypeptide with phase behavior, (ii) an mRNA substrate, and (iii) poly(adenylate)nucleotide results in the formation of a concentrated droplet. In some embodiments, the polyadenylate polymerase adds the poly(adenylate) nucleotide to the mRNA substrate in the concentrated droplet.

In some embodiments, the method comprises incubating a fusion protein comprising an enzyme and a polypeptide with phase behavior with a substrate. In some embodiments, the fusion protein is incubated with the substrate for between about 10 minutes to about 24 hours, for example, about 10 minutes, about 15 minutes, about 20 minutes, about 25 minutes, about 30 minutes, about 35 minutes, about 40 minutes, about 45 minutes, about 50 minutes, about 55 minutes, or about 60 minutes about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours, including all ranges in between. In some embodiments, the fusion protein is incubated with the substrate for between about 1 and about 4 hours. In some embodiments, the fusion protein is incubated with the substrate for between about 10 minutes and about 1 hour.

In some embodiments, incubation occurs at a temperature between about 10° C. and about 80° C. for example, at about 10° C., 15° C., 16° C., 20° C., 25° C., 30° C., 35° C., 37° C., about 40° C., about 45° C., about 50° C., about 55° C., about 60° C., about 65° C., about 70° C., about 75° C., or about 80° C.

In some embodiments, introduction of an environmental factor provides the enzyme of a fusion protein comprising a polypeptide with phase behavior access to the substrate. For example, introduction of an environmental factor solubilizes the fusion protein and allows reaction between an enzyme and substrate to occur. In some embodiments, introduction of an environmental factor prevents the enzyme's access to the substrate. For example, introduction of an environmental factor may precipitate the fusion protein.

In some embodiments, a fusion protein is separated from the substrate. In some embodiments, separation occurs on the basis of size and/or density, for example, based on molecular weight or diameter. In some embodiments, any of the techniques described herein for separating molecules on the basis of size may be applied to separate a fusion protein from the substrate. In some embodiments, continuous centrifugation, tangential flow filtration, or centrifugation are used to separate a fusion protein from the substrate on the basis of size.

In some embodiments, the multi-step enzymatic process is in vitro transcription, and the substrate is DNA. In some embodiments, the DNA is linear DNA or circular DNA. In some embodiments, the first enzyme of the first fusion protein comprises RNA polymerase. In some embodiments, the RNA polymerase is T7, T3, or SP6 RNA polymerase. In some embodiments, the first fusion protein is added to a composition comprising the DNA substrate. In some embodiments, the composition also comprises ribonucleotide triphosphates and/or a buffer (e.g., a buffer comprising dithiothreitol and magnesium). In some embodiments, an environmental factor is added to provide the first enzyme (e.g., RNA polymerase) access to the DNA substrate. In some embodiments, the fusion protein and DNA substrate are incubated for about 10 minutes to about 24 hours. In some embodiments, incubation of RNA polymerase with a DNA substrate results in the production of RNA. In some embodiments, the first fusion protein is separated from the substrate via application of an environmental factor. In some embodiments, any method described herein for separating molecules on the basis of size is used to separate the RNA from the first fusion protein.

In some embodiments, the RNA produced after reaction with the first enzyme of the first fusion protein is used as a substrate for additional enzymatic reactions. For example, in some embodiments, a second fusion protein comprising the second enzyme mRNA Cap 2′-O-Methyltransferase and a polypeptide with phase behavior is provided to the RNA. This reaction results in the RNA containing a methyl group at the 5′ end. In some embodiments, the second fusion protein and RNA substrate are incubated for about 10 minutes to about 24 hours. In some embodiments, the second fusion protein is separated from the substrate via application of an environmental factor. In some embodiments, any method described herein for separating molecules on the basis of size is used to separate the capped mRNA from the second fusion protein.

In some embodiments, a second fusion protein comprising the second enzyme polyadenylate polymerase is added to an RNA substrate. In some embodiments, the composition comprising the RNA substrate comprises poly(adenylate)nucleotide. In some embodiments, the composition comprising the RNA comprises polyadenylate polymerase. Polyadenylate polymerase catalyzes the addition of a poly(A) tail to RNA. In some embodiments, the second fusion protein and RNA substrate are incubated for about 10 minutes to about 24 hours. In some embodiments, the second fusion protein is separated from the substrate via application of an environmental factor. In some embodiments, any method described herein for separating molecules on the basis of size is used to separate the RNA containing a poly(A) tail from the second fusion protein.

In some embodiments, a second fusion protein comprising the second enzyme PABP is added to an RNA substrate. In some embodiments, the composition comprising the RNA substrate comprises poly(adenylate)nucleotide. In some embodiments, the composition comprising the RNA comprises polyadenylate polymerase. PABP assists with the addition of a poly(A) tail to RNA. In some embodiments, the second fusion protein and RNA substrate are incubated for about 10 minutes to about 24 hours. In some embodiments, the second fusion protein is separated from the substrate via application of an environmental factor. In some embodiments, any method described herein for separating molecules on the basis of size is used to separate the RNA containing a poly(A) tail from the second fusion protein.

### Environmental Factors

In some embodiments, the methods of the disclosure provide one or more environmental factors to a composition comprising a fusion protein described herein. In some embodiments, one or more environmental factors are applied to reversibly aggregate the fusion protein. Application of an environmental factor causes a change in the composition comprising the fusion protein. In some embodiments, an environmental factor is used to reversibly aggregate the fusion protein. In some embodiments, an environmental factor is used to reversibly disaggregate the fusion protein. In some embodiments, the environmental factor is used to separate a fusion protein from one or more impurities in the composition comprising the fusion protein. In some embodiments, the one or more environmental factors cause the size of the fusion protein aggregates to increase. In some embodiments, the one or more environmental factors enables the first polypeptide of the fusion protein to retain its structure, function, and activity. In some embodiments, the one or more environmental factors enables the first polypeptide of the fusion protein to enhance its native structure, function, and activity.

In some embodiments, the methods of the disclosure comprise applying at least 1, at least 2, at least 3, at least 4, at least 5, at least 6, at least 7, at least 8, at least 9, or at least 10 environmental factors to a composition comprising at least 1, at least 2, at least 3, at least 4, at least 5, at least 6, at least 7, at least 8, at least 9, or at least 10 fusion proteins.

In some embodiments, the environmental factor is a change in temperature. In some embodiments, the temperature is increased by about 0.5° C., about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., or about 40° C. In some embodiments, the temperature is decreased by about 0.5° C., about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., or about 40° C.

In some embodiments, the environmental factor is a change in pH. In some embodiments, the pH is increased by about 0.1, about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, or about 6.0 units.

In some embodiments, the pH is decreased by about 0.1, about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, or about 6.0 units.

In some embodiments, the environmental factor is change in ionic strength. In some embodiments, the change in ionic strength is brought about by increasing the concentration of salt. In some embodiments, the change in ionic strength is brought about by decreasing the concentration of salt. Non-limiting examples of salts include sodium chloride, potassium chloride, magnesium chloride, calcium chloride ammonium chloride, sodium acetate, sodium citrate, copper sulfate, sodium iodide, and sodium sulfate. In some embodiments, the salt has a concentration of between about 0.1 M and about 5 M, for example, about 0.1 M, about 0.2 M, about 0.3 M, about 0.4 M, about 0.5 M, about 0.6 M, about 0.7 M, about 0.8 M, about 0.9 M, about 1 M, about 1.1 M, about 1.2 M, about 1.3 M, about 1.4 M, about 1.5 M, about 1.6 M, about 1.7 M, about 1.8 M, about 1.9 M, about 2 M, about 2.1 M, about 2.2 M, about 2.3 M, about 2.4 M, about 2.5 M, about 2.6 M, about 2.7 M, about 2.8 M, about 2.9 M, about 3 M, about 3.1 M, about 3.2 M, about 3.3 M, about 3.4 M, about 3.5 M, about 3.6 M, about 3.7 M, about 3.8 M, about 3.9 M, about 4 M, about 4.1 M, about 4.2 M, about 4.3 M, about 4.4 M, about 4.5 M, about 4.6 M, about 4.7 M, about 4.8 M, about 4.9 M, or about 5 M. In some embodiments, the salt has a concentration of 0.6 M. In some embodiments, dialysis is used to change the concentration of salt in the composition comprising the protein-based purification matrix and biologic, contaminant, and/or molecule.

In some embodiments, the environmental factor is the addition of a cofactor. Non-limiting examples of cofactors include calcium, magnesium, cobalt, copper, zinc, iron, manganese, selenium, molybdenum, potassium, coenzyme A (CoA), a nucleoside triphosphate, and a vitamin (e.g., vitamin A, B, C, D, or F). In some embodiments, the cofactor is calcium. In some embodiments, the nucleoside triphosphate is adenosine triphosphate, uridine triphosphate, guanosine triphosphate, cytidine triphosphate, or thymidine triphosphate. In some embodiments, the vitamin is a fat-soluble. In some embodiments, the vitamin is water-soluble. Non-limiting examples of vitamins include vitamin A, vitamin B1 (thiamine), vitamin B2 (riboflavin), vitamin B3 (niacin or niacinamide), vitamin B5 (pantothenic acid), Vitamin B6 (pyridoxine, pyridoxal, or pyridoxamine, or pyridoxine hydrochloride), vitamin B7 (biotin), vitamin B9 (folic acid), vitamin B12, vitamin C, vitamin D, Vitamin E, vitamin K, K1, and K2, folic acid, and biotin.

In some embodiments, the environmental factor is a change in the concentration of the protein-based purification matrix. In some embodiments, the environmental factor is a change in the concentration of the biologic, contaminant, and/or molecule.

In some embodiments, the environmental factor is a change in pressure of the composition comprising the protein-based purification matrix and biologic, contaminant, and/or molecule. In some embodiments, a change in pressure can be effected by increasing or decreasing the volume of the composition.

In some embodiments, the environmental factor is the addition of one or more surfactants. In some embodiments, the one or more surfactants are free fatty acid salts, soaps, fatty acid sulfonates, such as sodium lauryl sulfate, ethoxylated compounds, such as ethoxylated propylene glycol, lecithin, polygluconates, quaternary ammonium salts, lignin sulfonates, 3-((3-cholamidopropyl) dimethylammonio)-1-propanesulfonate (CHAPS), sugars, including sucrose and glucose, Triton X-100, and NP-40. In some embodiments, the surfactant is anionic, nonionic, or amphoteric.

In some embodiments, the environmental factor is the addition of one or more molecular crowding agents. Non-limiting examples of molecular crowding agents include polyethylene glycol, dextran, and ficoll. PEGS may include PEG400, PEG1450, PEG3000, PEG8000, and PEG10000.

In some embodiments, the environmental factor is the addition of one or more oxidizing agents. Non-limiting examples of oxidizing agents include hydrogen peroxide, hydrophilically or hydrophobically activated hydrogen peroxide, preformed peracids, monopersulfate or hypochlorite.

In some embodiments, the environmental factor is the addition of one or more reducing agents. In some embodiments, the one or more reducing agents is selected from the group consisting of dithiothreitol (DTT), 2-mercaptoethanol (BME), Tris (2-carboxyethyl) phosphine (TCEP), hydrazine, boron hydrides, amine boranes, lower alkyl substituted amine boranes, triethanolamine, and N,N,N′,N′-tetramethylethylenediamine (TEMED). In some embodiments, the environmental factor is the addition of one or more denaturing agents. Non-limiting examples of denaturing agents include urea, guanidine hydrochloride, guanidine, sodium salicylate, dimethyl sulfoxide, and propylene glycol.

In some embodiments, the environmental factor is the addition of one or more enzymes. Non-limiting examples of enzymes include proteases, kinases, phosphatases, synthetases, transferases, nucleases such as restriction endonucleases, lyases, isomerases, dehydrogenases, decarboxylases, and lipases.

In some embodiments, the environmental factor is the application of electromagnetic waves. In some embodiments, the environmental factor is the application of light. In some embodiments, the electromagnetic waves have a wavelength between about 0.0001 nm and about 100 m. In some embodiments, the electromagnetic waves are selected from the group consisting of gamma rays, x-rays, ultraviolet, visible, infrared, and radio waves. In some embodiments, the electromagnetic waves are gamma rays. In some embodiments, the gamma rays have a wavelength between about 0.0001 nm and about 0.01 nm, e.g. 0.0001 nm, 0.0005 nm, 0.001 nm, 0.002 nm, 0.003 nm, 0.004 nm, 0.005 nm, 0.006 nm, 0.007 nm, 0.008 nm, 0.009 nm, and 0.01 nm. In some embodiments, the x-rays have a wavelength between about 0.01 nm and about 10 nm, e.g. about 0.01 nm, 0.02 nm, 0.03 nm, 0.04 nm, 0.05 nm, 0.06 nm, 0.07 nm, 0.08 nm, 0.09 nm, 0.10 nm, 0.2 nm, 0.3 nm, 0.4 nm, 0.5 nm, 0.6 nm, 0.7 nm, 0.8 nm, 0.9 nm, 1 nm, 2 nm, 3 nm, 4 nm, 5 nm, 6 nm, 7 nm, 8 nm, 9 nm, or about 10 nm. In some embodiments, the ultraviolet radiation has a wavelength between about 10 nm about 400 nm, e.g. about 10 nm, about 20 nm, about 30 nm, about 40 nm, about 50 nm, about 60 nm, about 70 nm, about 80 nm, about 90 nm, about 100 nm, about 150 nm, about 200 nm, about 250 nm, about 280 nm, about 300 nm, about 350 nm, or about 400 nm. In some embodiments, the visible waves have a wavelength of between about 400 nm and about 800 nm, e.g. about 400 nm, about 450 nm, about 500 nm, about 550 nm, about 600 nm, about 650 nm, about 700 nm, about 750 nm, or about 800 nm. In some embodiments, the infrared radiation has a wavelength of between about 800 nm and about 0.1 cm, e.g. about 800 nm, about 1 μm, about 2 μm, about 3 μm, about 4 μm, about 5 μm, about 6 μm, about 7 μm, about 8 μm, about 9 μm, about 10 μm, about 20 μm, about 30 μm, about 40 μm, about 50 μm, about 60 μm, about 70 μm, about 80 μm, about 90 μm, about 100 μm, about 200 μm, about 300 μm, about 400 μm, about 500 μm, about 600 μm, about 700 μm, about 800 μm, about 900 μm, or about 0.1 cm. In some embodiments, the radio waves have a wavelength of between about 0.1 cm and 100 m, e.g. about 0.1 cm, about 1 cm, about 10 cm, about 100 cm, about 1000 cm, about 2000 cm, about 3000 cm, about 4000 cm, about 5000 cm, about 6000 cm, about 7000 cm, about 8000 cm, about 9000 cm, or about 100 m.

All patents, patent applications, references, and journal articles cited in this disclosure are expressly incorporated herein by reference in their entireties for all purposes.

### EXAMPLES

### Example 1. Expression of a Fusion Protein Comprising the Binding Domain of the AAV Receptor and a Second Polypeptide with Phase Behavior

A nucleic acid encoding the ectodomain of the AAV receptor (AAVR) was fused to a nucleic acid encoding a polypeptide with phase behavior ((GVGVPGLGVPGVGVPGLGVPGVGVP)₁₆ (SEQ ID NO:87)) and cloned into a pET24 plasmid. The plasmid was transformed into BL21 E. coli cells, and the cells were maintained under conditions that allowed for expression of the fusion protein.

The fusion protein was purified, aliquoted and formulated in PBS (as a control) or subjected to the following conditions known to impact protein function: (1) lyophilization and resuspension in PBS, (2) 30 min incubation in 0.1M NaOH followed by neutralization and buffer exchange into PBS, (3) 30 min incubation in 6M guanidine hydrochloride (GuHCl) followed by buffer exchange into PBS, (4) 30 min incubation in PBS at 95° C. followed by cooling on ice and returning to room temperature. The activity of each sample (i.e., the ability of AAVR to bind its target) was then measured by an assay for AAV8 capture. Briefly, activity was defined as the percentage of AAV8 captured, quantified using a Progen AAV8 total capsid ELISA.

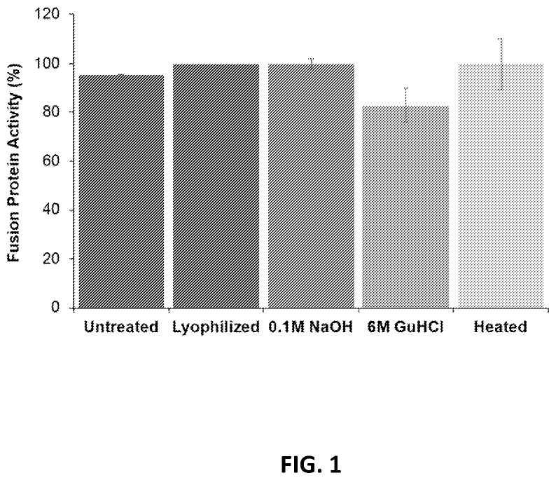

Results are shown in FIG. 1. For all treatment conditions, the activity of the fusion protein remained within 80% of the untreated control.

### Example 2: Expression of a Fusion Protein Comprising the Z Domain of Staphylococcal Protein a and a Second Polypeptide with Phase Behavior

A nucleic acid encoding the Z domain derived from staphylococcal protein A (Seq ID: 180), which is known to bind to antibodies, was fused to a nucleic acid encoding a polypeptide with phase behavior ((GVGVPGLGVPGVGVPGLGVPGVGVP)₁₆ (SEQ ID NO:87)) and cloned into a pET24 plasmid. The plasmid was transformed into BL21 E. coli cells, and the cells were maintained under conditions that allowed for expression of the fusion protein. The fusion protein was then purified, and lyophilized.

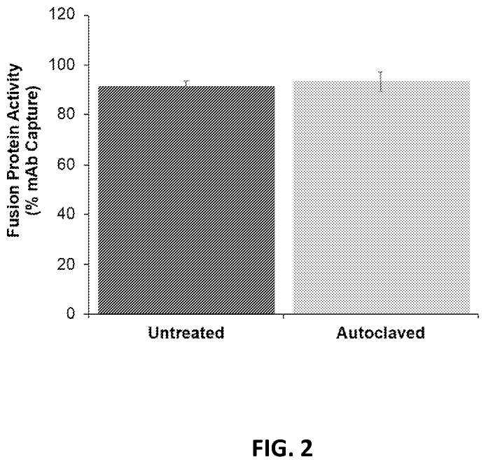

The fusion protein was autoclaved on gravity cycle, and resuspended in PBS. The activity of the protein A was then tested. Specifically, activity was measured using an assay for antibody capture. Results are shown in FIG. 2. Fusion to the polypeptide with phase behavior helped the protein A to retain at least 85% activity after autoclaving/resuspension in PBS, compared to unautoclaved control.

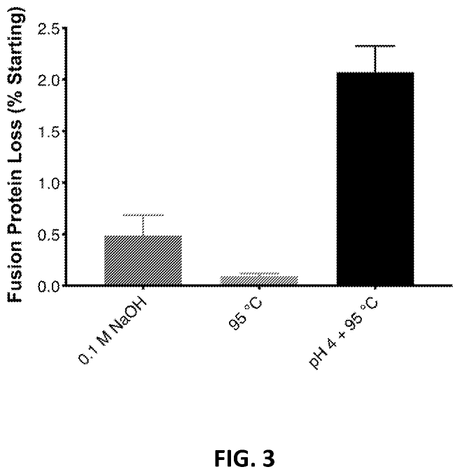

The protein was also tested to determine whether fusion to the polypeptide with phase behavior could prevent loss of soluble, folded protein A after treatment with 0.1 M NaOH, heating to 95° C. in PBS, or heating in acidic buffer at pH 4. For the given treatment conditions, the samples were incubated for 10-30 minutes and then neutralized or cooled and centrifuged to remove any aggregated protein. To measure the percentage of protein lost by treatment, total protein was measured using a Protein A ELISA assay and compared to an untreated control. As shown in FIG. 3, exposure to these conditions resulted in less than 2% loss.

### Example 3. Expression of a Fusion Protein Comprising the Binding Domain of the AAV Receptor and a Second Polypeptide with Phase Behavior

A nucleic acid encoding PKD2 of the AAV receptor (Cys-His-PKD2) was fused to a nucleic acid encoding a polypeptide with phase behavior (ELP) and cloned into a pET24 plasmid. As a control, a nucleic acid encoding the PKD2 of the AAV receptor was cloned into a pET24 plasmid. The nucleic acid and protein sequences of PKD2 of the AAV receptor and the polypeptide with phase behavior are contained in Table 5 below.

TABLE 5
Nucleic Acid and Protein Sequences

| Target | | |
|---|---|---|
| Protein | Nucleic Acid Sequence | Protein Sequence |
| Cys-His- | ATGTGTCATCATCACCATCACCACAAT | (M)CHHHHHH-- |
| PKD2 | CGGCCACCAATTGCGATCGTGTCACCG | NRPPIAIVSPQFQEISLPTTSTVIDGSQST |
| | CAGTTCCAAGAAATCTCTTTACCGACA | DDDKIVQYHWEELKGPLREEKISEDTAILK |
| | ACTTCTACTGTCATTGATGGCAGCCAG | LSKLVPGNYTFSLTVVDSDGATNSTTANLT |
| | AGCACCGATGATGATAAAATCGTCCAG | VNKAVDYPGY |
| | TACCACTGGGAGGAATTGAAAGGGCCA | |
| | CTGCGCGAAGAAAAAATTAGCGAGGAC | |
| | ACTGCCATTCTTAAACTGAGTAAGCTC | |
| | GTACCGGGGAACTACACGTTCTCTTTG | |
| | ACAGTAGTCGATTCGGATGGGGCGACC | |
| | AACTCCACAACGGCGAATCTGACAGTT | |
| | AACAAGGCTGTGGATTATCCGggctac | |
| | tga | |
| ELP | GTGGGCGTTCCGGGGCTGGGTGTTCCG | VGVPGLGVPGVGVPGLGVPGVGVPGVGVPG |
| | GGCGTCGGTGTGCCAGGTCTGGGTGTA | LGVPGVGVPGLGVPGVGVPGVGVPGLGVPG |
| | CCGGGTGTTGGCGTCCCTGGTGTGGGC | VGVPGLGVPGVGVPGVGVPGLGVPGVGVPG |
| | GTTCCGGGGCTGGGTGTTCCGGGCGTC | LGVPGVGVPGVGVPGLGVPGVGVPGLGVPG |
| | GGTGTGCCAGGTCTGGGTGTACCGGGT | VGVPGVGVPGLGVPGVGVPGLGVPGVGVPG |
| | GTTGGCGTCCCTGGTGTGGGCGTTCCG | VGVPGLGVPGVGVPGLGVPGVGVPGVGVPG |
| | GGGCTGGGTGTTCCGGGCGTCGGTGTG | LGVPGVGVPGLGVPGVGVPGVGVPGLGVPG |
| | CCAGGTCTGGGTGTACCGGGTGTTGGC | VGVPGLGVPGVGVPGVGVPGLGVPGVGVPG |
| | GTCCCTGGTGTGGGCGTTCCGGGGCTG | LGVPGVGVPGVGVPGLGVPGVGVPGLGVPG |
| | GGTGTTCCGGGCGTCGGTGTGCCAGGT | VGVPGVGVPGLGVPGVGVPGLGVPGVGVPG |
| | CTGGGTGTACCGGGTGTTGGCGTCCCT | VGVPGLGVPGVGVPGLGVPGVGVPGVGVPG |
| | GGTGTGGGCGTTCCGGGGCTGGGTGTT | LGVPGVGVPGLGVPGVGVPGVGVPGLGVPG |
| | CCGGGCGTCGGTGTGCCAGGTCTGGGT | VGVPGLGVPGVGVPGVGVPGLGVPGVGVPG |
| | GTACCGGGTGTTGGCGTCCCTGGTGTG | LGVPGVGVPG |
| | GGCGTTCCGGGGCTGGGTGTTCCGGGC | |
| | GTCGGTGTGCCAGGTCTGGGTGTACCG | |
| | GGTGTTGGCGTCCCTGGTGTGGGCGTT | |
| | CCGGGGCTGGGTGTTCCGGGCGTCGGT | |
| | GTGCCAGGTCTGGGTGTACCGGGTGTT | |
| | GGCGTCCCTGGTGTGGGCGTTCCGGGG | |
| | CTGGGTGTTCCGGGCGTCGGTGTGCCA | |
| | GGTCTGGGTGTACCGGGTGTTGGCGTC | |
| | CCTGGTGTGGGCGTTCCGGGGCTGGGT | |
| | GTTCCGGGCGTCGGTGTGCCAGGTCTG | |
| | GGTGTACCGGGTGTTGGCGTCCCTGGT | |
| | GTGGGCGTTCCGGGGCTGGGTGTTCCG | |
| | GGCGTCGGTGTGCCAGGTCTGGGTGTA | |
| | CCGGGTGTTGGCGTCCCTGGTGTGGGC | |
| | GTTCCGGGGCTGGGTGTTCCGGGCGTC | |
| | GGTGTGCCAGGTCTGGGTGTACCGGGT | |
| | GTTGGCGTCCCTGGTGTGGGCGTTCCG | |
| | GGGCTGGGTGTTCCGGGCGTCGGTGTG | |
| | CCAGGTCTGGGTGTACCGGGTGTTGGC | |
| | GTCCCTGGTGTGGGCGTTCCGGGGCTG | |
| | GGTGTTCCGGGCGTCGGTGTGCCAGGT | |
| | CTGGGTGTACCGGGTGTTGGCGTCCCT | |
| | GGTGTGGGCGTTCCGGGGCTGGGTGTT | |
| | CCGGGCGTCGGTGTGCCAGGTCTGGGT | |
| | GTACCGGGTGTTGGCGTCCCTGGTGTG | |
| | GGCGTTCCGGGGCTGGGTGTTCCGGGC | |
| | GTCGGTGTGCCAGGTCTGGGTGTACCG | |
| | GGTGTTGGCGTCCCTGGTGTGGGCGTT | |
| | CCGGGGCTGGGTGTTCCGGGCGTCGGT | |
| | GTGCCAGGTCTGGGTGTACCGGGTGTT | |
| | GGCGTCCCTGGG | |
| Cys-His- | ATGTGTCATCATCACCATCACCACGTG | (M)CHHHHHH-- |
| 40L80- | GGCGTTCCGGGGCTGGGTGTTCCGGGC | VGVPGLGVPGVGVPGLGVPGVGVPGVGVPG |
| PKD2 | GTCGGTGTGCCAGGTCTGGGTGTACCG | LGVPGVGVPGLGVPGVGVPGVGVPGLGVPG |
| (Fusion | GGTGTTGGCGTCCCTGGTGTGGGCGTT | VGVPGLGVPGVGVPGVGVPGLGVPGVGVPG |
| Protein) | CCGGGGCTGGGTGTTCCGGGCGTCGGT | LGVPGVGVPGVGVPGLGVPGVGVPGLGVPG |
| | GTGCCAGGTCTGGGTGTACCGGGTGTT | VGVPGVGVPGLGVPGVGVPGLGVPGVGVPG |
| | GGCGTCCCTGGTGTGGGCGTTCCGGGG | VGVPGLGVPGVGVPGLGVPGVGVPGVGVPG |
| | CTGGGTGTTCCGGGCGTCGGTGTGCCA | LGVPGVGVPGLGVPGVGVPGVGVPGLGVPG |
| | GGTCTGGGTGTACCGGGTGTTGGCGTC | VGVPGLGVPGVGVPGVGVPGLGVPGVGVPG |
| | CCTGGTGTGGGCGTTCCGGGGCTGGGT | LGVPGVGVPGVGVPGLGVPGVGVPGLGVPG |
| | GTTCCGGGCGTCGGTGTGCCAGGTCTG | VGVPGVGVPGLGVPGVGVPGLGVPGVGVPG |
| | GGTGTACCGGGTGTTGGCGTCCCTGGT | VGVPGLGVPGVGVPGLGVPGVGVPGVGVPG |
| | GTGGGCGTTCCGGGGCTGGGTGTTCCG | LGVPGVGVPGLGVPGVGVPGVGVPGLGVPG |
| | GGCGTCGGTGTGCCAGGTCTGGGTGTA | VGVPGLGVPGVGVPGVGVPGLGVPGVGVPG |
| | CCGGGTGTTGGCGTCCCTGGTGTGGGC | LGVPGVGVPGNRPPIAIVSPQFQEISLPTT |
| | GTTCCGGGGCTGGGTGTTCCGGGCGTC | STVIDGSQSTDDDKIVQYHWEELKGPLREE |
| | GGTGTGCCAGGTCTGGGTGTACCGGGT | KISEDTAILKLSKLVPGNYTFSLTVVDSDG |
| | GTTGGCGTCCCTGGTGTGGGCGTTCCG | ATNSTTANLTVNKAVDYPGY |
| | GGGCTGGGTGTTCCGGGCGTCGGTGTG | |
| | CCAGGTCTGGGTGTACCGGGTGTTGGC | |
| | GTCCCTGGTGTGGGCGTTCCGGGGCTG | |
| | GGTGTTCCGGGCGTCGGTGTGCCAGGT | |
| | CTGGGTGTACCGGGTGTTGGCGTCCCT | |
| | GGTGTGGGCGTTCCGGGGCTGGGTGTT | |
| | CCGGGCGTCGGTGTGCCAGGTCTGGGT | |
| | GTACCGGGTGTTGGCGTCCCTGGTGTG | |
| | GGCGTTCCGGGGCTGGGTGTTCCGGGC | |
| | GTCGGTGTGCCAGGTCTGGGTGTACCG | |
| | GGTGTTGGCGTCCCTGGTGTGGGCGTT | |
| | CCGGGGCTGGGTGTTCCGGGCGTCGGT | |
| | GTGCCAGGTCTGGGTGTACCGGGTGTT | |
| | GGCGTCCCTGGTGTGGGCGTTCCGGGG | |
| | CTGGGTGTTCCGGGCGTCGGTGTGCCA | |
| | GGTCTGGGTGTACCGGGTGTTGGCGTC | |
| | CCTGGTGTGGGCGTTCCGGGGCTGGGT | |
| | GTTCCGGGCGTCGGTGTGCCAGGTCTG | |
| | GGTGTACCGGGTGTTGGCGTCCCTGGT | |
| | GTGGGCGTTCCGGGGCTGGGTGTTCCG | |
| | GGCGTCGGTGTGCCAGGTCTGGGTGTA | |
| | CCGGGTGTTGGCGTCCCTGGTGTGGGC | |
| | GTTCCGGGGCTGGGTGTTCCGGGCGTC | |
| | GGTGTGCCAGGTCTGGGTGTACCGGGT | |
| | GTTGGCGTCCCTGGTGTGGGCGTTCCG | |
| | GGGCTGGGTGTTCCGGGCGTCGGTGTG | |
| | CCAGGTCTGGGTGTACCGGGTGTTGGC | |
| | GTCCCTGGGAATCGGCCACCAATTGCG | |
| | ATCGTGTCACCGCAGTTCCAAGAAATC | |
| | TCTTTACCGACAACTTCTACTGTCATT | |
| | GATGGCAGCCAGAGCACCGATGATGAT | |
| | AAAATCGTCCAGTACCACTGGGAGGAA | |
| | TTGAAAGGGCCACTGCGCGAAGAAAAA | |
| | ATTAGCGAGGACACTGCCATTCTTAAA | |
| | CTGAGTAAGCTCGTACCGGGGAACTAC | |
| | ACGTTCTCTTTGACAGTAGTCGATTCG | |
| | GATGGGGCGACCAACTCCACAACGGCG | |
| | AATCTGACAGTTAACAAGGCTGTGGAT | |
| | TATCCGggctactga | |

A 5 mL overnight culture of BL21 E. coli cells was grown to express the His-tagged PKD2 protein, either alone or as a fusion with an ELP. 250 uL of this culture was used to inoculate a 50 mL culture, which was induced with 0.5 mM IPTG after 9 hours. After 24 hrs of incubation, the cells from each culture were centrifuged, resuspended in 10 mL of PBS buffer, and sonicated. The lysates were clarified by centrifugation. 1.5 mL of each clarified lysate was purified using a HisLink protein purification kit (Promega) and 200 uL elution fractions. The eluted material was quantified by measuring the 280 nm absorbance. Beer's Law was then used to calculate the grams and moles of protein and fusion protein purified per liter of E. coli cell culture. The purity and correct molecular weights were also assessed qualitatively using SDS-PAGE and Coomassie staining.

Expression of the fusion protein comprising PKD2 was compared to expression of PKD2 in the absence of polypeptide with phase behavior.

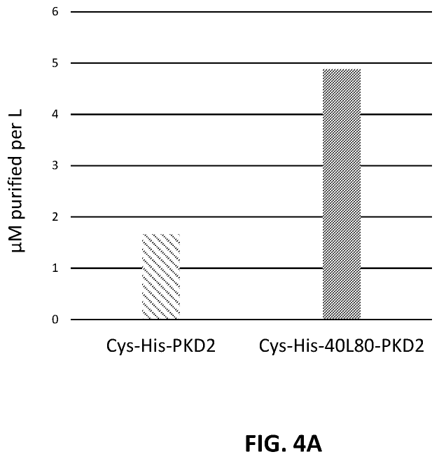

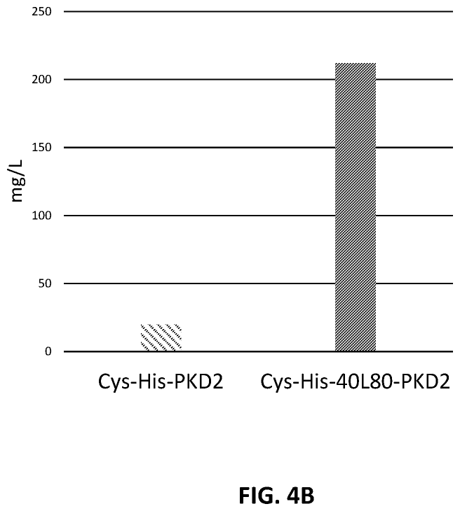

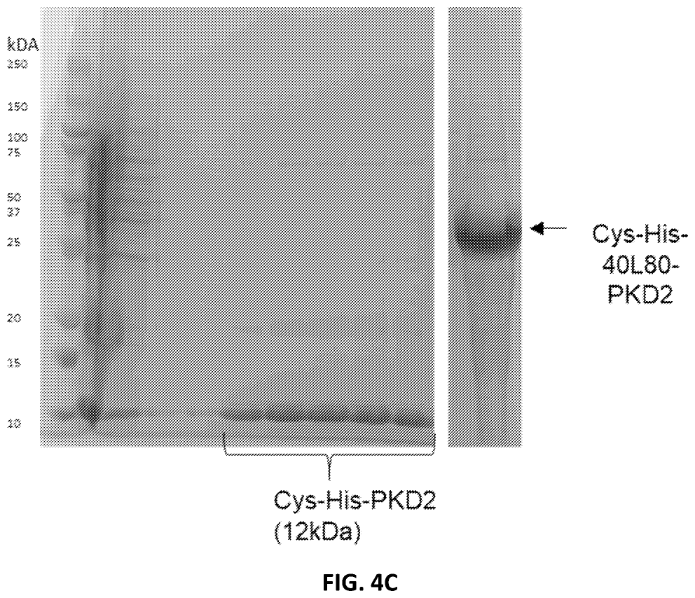

FIG. 4A shows the concentrations of PKD2 and fusion protein comprising PKD2 purified per liter. FIG. 4B shows the amount of PKD2 and fusion protein comprising PKD2 purified per liter. FIG. 4C shows expression of PKD2 and the fusion protein comprising PKD2 on a gel. These results show that ten times more soluble protein mass (fusion protein) and over three times more PKD2 (first polypeptide) can be extracted when the PKD2 domain is fused to a polypeptide with phase behavior compared to when PKD2 is expressed without a polypeptide with phase behavior.

### Example 4. Expression of a Fusion Protein Comprising the Binding Domain of the AAV Receptor and a Second Polypeptide with Phase Behavior

A nucleic acid encoding the CR3 domain of the LDL receptor (LDLR) was fused to a nucleic acid encoding a polypeptide with phase behavior and cloned into a pET24 plasmid. The plasmid was transformed into BL21 E. coli cells, and the cells were maintained under conditions that allowed for expression of the fusion protein. The fusion protein is referred to as IsoTag-LV in this example. The amino acid sequence of IsoTag-LV is found in Table 6 below.

TABLE 6
IsoTag-LV Protein Sequence

(M)SKGPGVGVPGVGVPGVGVPGVGVPGVGVPGVGVPGVGVPGVGVP
GVGVPGVGVPGVGVPGVGVPGVGVPGVGVPGVGVPGVGVPGVGVPGV
GVPGVGVPGVGVPGVGVPGVGVPGVGVPGVGVPGVGVPGVGVPGVGV
PGVGVPGVGVPGVGVPGVGVPGVGVPGVGVPGVGVPGVGVPGVGVPG
VGVPGVGVPGVGVPGVGVPGVGVPGVGVPGVGVPGVGVPGVGVPGVG
VPGVGVPGVGVPGVGVPGVGVPGVGVPGVGVPGVGVPGVGVPGVGVP
GVGVPGVGVPGVGVPGVGVPGVGVPGVGVPGVGVPGVGVPGVGVPGV
GVPGVGVPGVGVPGVGVPGVGVPGVGVPGVGVPGVGVPGVGVPGVGV
PGVGVPGVGVPGVGVPGVGVPGVGVPGVGVPGTCSQDEFRCHDGKCI
SRQFVCDSDRDCLDGSDEASCPG

The stability of IsoTag-LV was evaluated by measuring lentivirus (LV) capture activity of the fusion protein control versus the fusion protein with exposure to extreme conditions known to degrade, aggregate, or inactivate polypeptides.

20 μM IsoTag-LV was incubated for 30 minutes in 6M guanidine-HCL (6M GuHCl) or 0.1M NaOH (0.1M NaOH) followed by six, 500 μL PBS washes in 30 kDa Amicon spin filters. 20 μM IsoTag-LV was incubated for 30 minutes at 95° C., followed by a 30-minute incubation on ice.

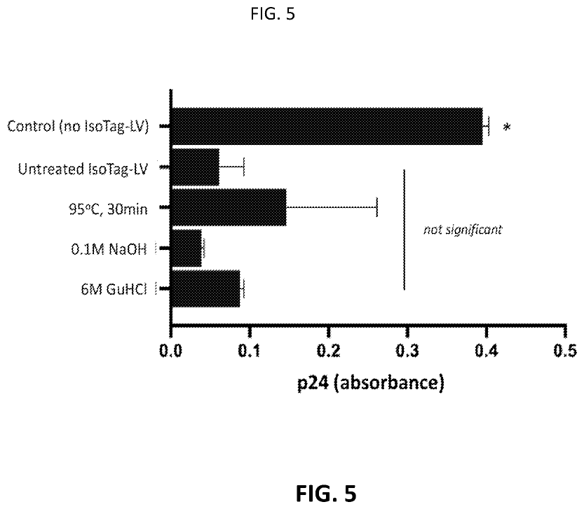

10 μM IsoTag from the described incubations, as well as untreated IsoTag (Control IsoTag-LV) were added to 500 μL lentivirus, incubated for 5 minutes on ice and then 5 minutes at 37° C. before being spun for 5 minutes at 10,000 rpm to capture and pellet the lentivirus target. Harvested supernatants (representing any uncaptured LV target) were analyzed using a p24 assay (VPK-107, Cell Biolabs), to measure LV envelope p24 protein as a surrogate measure of lentivirus titer. As a control for no capture activity, lentivirus without any IsoTag addition was also measured (Control (No IsoTag-LV) in duplicate. Absorbance values were plotted using GraphPad Prism V9. Error bars represent the standard error of the mean. There is no statistically significant difference in the LV capture activity of the control fusion protein compared to the fusions treated with heating to 95° C., 0.1M NaOH incubation, or 6M GuHCl incubation. This shows that expression the CR3 domain of LDLR as a fusion protein with a polypeptide with phase behavior protects the CR3 domain of LDLR from degradation. Results are shown in FIG. 5.

### Example 5: Estimated Yield in E. coli of Additional Fusion Proteins Comprising a First Polypeptide and a Second Polypeptide with Phase Behavior

Nucleic acids encoding various proteins with biological functionality (referred to herein as target proteins) were fused to nucleic acid sequences encoding a polypeptide with phase behavior ((GVGVPGLGVPGVGVPGLGVPGVGVP)₁₆ (SEQ ID NO:87), and expressed as recombinant fusion proteins in E. coli BL21 cells. More specifically, the nucleic acids encoding a fusion protein were cloned into a pET24 plasmid, which is transfected into the E. coli cells. Expression of the fusion proteins was induced by adding Isopropyl β-d-1-thiogalactopyranoside (IPTG) to a shake flask containing the cells. The fusion proteins were purified using phase separation, indicating that the yield was at least 10 mg/L. Table 7 shows a list of target proteins for use in the fusions. It is estimated that the yield of each purified protein may be at least 15 mg per liter, with some yields as high as 300 mg per liter.

TABLE 7
Target protein expression levels

| | Expressed in Soluble Fraction? |
|---|---|
| Target Protein | (Yes(Y)/No(N)) |
| SpA Z-domain | Y |
| B domain of Protein A | Y |
| C domain of Protein A | Y |
| Binding domain of AAVR | Y |
| Full length AAVR | Y |
| CD4 | Y |
| CAR | Y |
| LDL-R domain | Y |
| ABD | Y |
| FN3 | Y |
| PABP | Y |
| ZBP | Y |

### Example 6: Use of Fusion Proteins for Modification of Nucleic Acids

This example evaluates the ability of the fusion proteins described herein to facilitate modification of DNA and RNA. A fusion protein comprising a nucleic acid binding protein (NBP) and a polypeptide with phase behavior is incubated with a nucleic acid substrate (e.g., DNA or RNA) for sufficient time for the NBP in either the soluble or phase separated form to generate a product or to generate an affinity bound complex. Subsequently, an environmental factor (e.g., 0.6 M NaCl) is added to the solution to phase separate the fusion protein. This phase separation serves to increase the enzymatic reaction, concentration, and/or purity of the nucleic acid target. The fusion protein is separated from the product via tangential flow filtration, depth filtration, or centrifugation. The product is incubated with additional fusion proteins and subjected to the same procedures as described for the first fusion protein to make additional modifications to the nucleic acid. In each of the aforementioned steps, additional reagents may be added to facilitate performance of the fusion protein (e.g., cofactors, ribonucleotide triphosphates, deoxynucleoside triphosphates, salts, etc.)

At any point, the samples can be centrifuged or filtered to separate the fusion protein and anything affinity bound to it from the components in the soluble phase. At any point, a second environmental factor (e.g. low pH, cofactor, salt) can be added to disrupt binding of the product or substrate with the NBP fusion protein in its soluble or phase separated state.

An exemplary process for transcribing DNA to mRNA is described below. A nucleic acid encoding the NBP T7 RNA polymerase and a polypeptide with phase behavior (e.g., GVGVPGLGVPGVGVPGLGVPGVGVP)₁₆ (SEQ ID NO: 87)) is expressed as a recombinant fusion protein in E. coli BL21 cells and purified. The purified fusion protein is incubated with a composition comprising a DNA substrate and rNTPs at conditions in which the fusion protein is soluble for about an hour at 37° C. to form RNA. Salt (0.6 M NaCl) is added to separate the fusion protein, which is bound to the RNA product, from the composition. The RNA product is separated from the fusion protein by continuous centrifugation.

Subsequent reactions are performed to add a poly(A) tail to the 3′ end of the RNA product and a methyl group to the 5′ end of the RNA product. The poly(A) tail is added via a fusion protein comprising PABP and a polypeptide with phase behavior. The methyl group is added to the 5′ end of the RNA product via a fusion protein comprising mRNA Cap 2-O-Methyltransferase and a polypeptide with phase behavior. The final product is a mRNA containing a 5′ methyl group and a 3′ poly(A) tail.

Additional modifications to DNA and RNA may be performed in a similar manner using any of the NBP described herein. For example, the NBP may be any of the following proteins: T7 RNA polymerase, Rnase inhibitor, 2′-O-Methyltransferase, Inorganic Pyrophosphatase, Poly(A) Polymerase. DNase I, Calf intestinal phosphatase, Antarctic phosphatase, D1 subunit of the Vaccinia virus mRNA capping enzyme, Guanine-7-methyltransferase (found in D1 subunit of the Vaccinia virus mRNA capping enzyme), Guanylyltransferase (found in D1 subunit of the Vaccinia virus mRNA capping enzyme), RNA triphosphatase (found in D1 subunit of the Vaccinia virus mRNA capping enzyme), and D12 subunit of vaccinia virus mRNA capping enzyme, a stem-loop binding protein, a heterogenous ribonucleoprotein (hnRNP), GroEL, Edc3, DHX9, Xrn1, Dep1, Dep2, LAF-1, MEG-1, MEG-3, ASF/SF2 splicing factor, serine/arginine rich splicing factor 4 (SRp75), the serine and arginine rich splicing factor 1 (SRSF1), the L3 ribosomal protein, the L4 ribosomal protein, the L13 ribosomal protein, the L20 ribosomal protein, the L22 ribosomal protein, the L24 ribosomal protein, the L24e ribosomal protein, the S12 ribosomal protein, the S14 ribosomal protein, and the eukaryotic initiation factor 4E-binding protein 1 (4EBP1), Tat, Rev, RSG-1.2 peptide, poly(A)-binding protein (PABP), eukaryotic translation initiation factor 4E (eIF4E), eukaryotic translation initiation factor3 Subunit D (eIF3D), heterogenous nuclear ribonucleoproteins (hnRNPs), RNA-specific adenosine deaminase 1 (ADAR1), RNA-specific adenosine deaminase 2 (ADAR2), CspB from Bacillus subtilis (Bscscp), Y-box protein 1 cold shock domain (YB1-CSD), a Fox-1 protein (FOX1), poly(A)-binding protein (PABP), Staufen protein, TIS11d, zinc finger protein (ZNF), Z-DNA binding protein 1 (ZBP1), retinoic acid-inducible gene-I (RIG-I) like protein, toll like receptor 7 (TLR7), toll like receptor 8 (TLR3), toll like receptor 8 (TLR8), retinoic acid-inducible gene I (RIG-I), melanoma differentiation-associated protein 5 (MDA5), interferon induced protein with tetratricopeptide repeats 1 (IFIT1), protein kinase R (PKR), an oligoadenylate synthase-like (OASL) protein (e.g., OAS1, OAS2, OAS3, or OASL), ribonuclease E (RNASE E), gamma-interferon-inducible protein Ifi-16 (IF116), cyclic GMP-AMP synthase (cGAS), or a catalytic fragment thereof.

### NUMBERED EMBODIMENTS OF THE DISCLOSURE

Notwithstanding the appended claims, the disclosure sets forth the following numbered embodiments:

1. A fusion protein comprising a first polypeptide and a second polypeptide, wherein the second polypeptide has phase behavior.

2. The fusion protein of embodiment 1, wherein the first polypeptide is:
i) an enzyme, or a derivative or catalytic fragment thereof;
ii) an antibody, or a derivative or antigen-binding fragment thereof;
iii) a signaling molecule, or a fragment or derivative thereof;
iv) a structural protein, or a fragment or derivative thereof; or
v) a hormone, or a fragment or derivative thereof.

3. The fusion protein of embodiment 1, wherein the first polypeptide is a mammalian polypeptide.

4. The fusion protein of embodiment 1, wherein the first polypeptide is a viral polypeptide.

5. The fusion protein of embodiment 1, wherein the first polypeptide is a bacterial polypeptide.

6. The fusion protein of embodiment 1, wherein the first polypeptide is a toxin.

7. The fusion protein of embodiment 1, wherein the first polypeptide is an antigenic polypeptide.

8. The fusion protein of embodiment 1, wherein the first polypeptide is an enzyme capable of performing one or more steps involved in protein synthesis or modification.

9. The fusion protein of embodiment 1, wherein the first polypeptide is an enzyme capable of performing one or more steps involved in DNA synthesis or modification.

10. The fusion protein of embodiment 1, wherein the first polypeptide is an enzyme capable of performing one or more steps involved in RNA synthesis or modification.

11. The fusion protein of embodiment 1, wherein the first polypeptide is selected from the cluster of differentiation 4 (CD4), the Z-domain of Staphylococcus protein A (SpA Z-domain), low-density lipoprotein receptor (LDLR), albumin binding polypeptide (ABD), coxsackievirus and adenovirus receptor (CAR), fibronectin type III (FN3), poly(A) binding protein (PABP), Z-DNA binding protein 1 (ZBP1), or a fragment or derivative thereof.

12. The fusion protein of embodiment 1, wherein the first polypeptide is a polypeptide isolated or derived from SARS-COV-2.

13. The fusion protein of embodiment 12, wherein the first polypeptide is the SARS-COV-2 spike protein, or a fragment or derivative thereof.

14. The fusion protein of embodiments 1-13, wherein the first polypeptide with phase behavior is an elastin-like polypeptide (ELP) or a resilin-like polypeptide (RLP).

15. The fusion protein of any one of embodiments 1-13, wherein the polypeptide with phase behavior comprises a pentapeptide repeat having the sequence (Val-Pro-Gly-Xaa-Gly)ₙ (SEQ ID NO: 10), or a randomized, scrambled analog thereof; wherein Xaa can be any amino acid except proline, wherein n is an integer between 1 and 360, inclusive of endpoints.

16. The fusion protein of any one of embodiments 1-13, wherein the polypeptide with phase behavior comprises an amino acid sequence selected from:

a.
(SEQ ID NO: 1)
(GRGDSPY)ₙ
b.
(SEQ ID NO: 2)
(GRGDSPH)ₙ
c.
(SEQ ID NO: 3)
(GRGDSPV)ₙ
d.
(SEQ ID NO: 4)
(GRGDSPYG)ₙ
e.
(SEQ ID NO: 5)
(RPLGYDS)ₙ
f.
(SEQ ID NO: 6)
(RPAGYDS)ₙ
g.
(SEQ ID NO: 7)
(GRGDSYP)ₙ
h.
(SEQ ID NO: 8)
(GRGDSPYQ)ₙ
i.
(SEQ ID NO: 9)
(GRGNSPYG)ₙ
j.
(SEQ ID NO: 11)
(GVGVP)ₙ;
k.
(SEQ ID NO: 12)
(GVGVPGLGVPGVGVPGLGVPGVGVP)ₘ;
l.
(SEQ ID NO: 13)
(GVGVPGVGVPGAGVPGVGVPGVGVP)ₘ;
m.
(SEQ ID NO: 14)
(GVGVPGWGVPGVGVPGWGVPGVGVP)ₘ;
n.
(SEQ ID NO: 15)
(GVGVPGVGVPGVGVPGVGVPGVGVPGVGVPGEGVPGFGV
PGVGVP)ₘ;
o.
(SEQ ID NO: 16)
(GVGVPGVGVPGVGVPGVGVPGVGVPGVGVPGKGVPGFGV
PGVGVP)ₘ;
and
p.
(SEQ ID NO: 17)
(GAGVPGVGVPGAGVPGVGVPGAGVP)ₘ;

or a randomized, scrambled analog thereof;
wherein:
n is an integer in the range of 20-360, inclusive of endpoints; and
m is an integer in the range of 4-25, inclusive of endpoints.

17. The fusion protein of any one of embodiments 1-13, wherein the polypeptide with phase behavior comprises an amino acid sequence selected from:

(SEQ ID NO: 143)
(GVGVP)ₘ;
(SEQ ID NO: 148)
(ZZPXXXXGZ)ₘ;
(SEQ ID NO: 149)
(ZZPXGZ)ₘ;
(SEQ ID NO: 150)
(ZZPXXGZ)ₘ;
or
(SEQ ID NO: 151)
(ZZPXXXGZ)ₘ,

wherein m is an integer between 10 and 160, inclusive of endpoints,
wherein X if present is any amino acid except proline or glycine, and
wherein Z if present is any amino acid.

18. The fusion protein of any one of embodiments 1-13, wherein the polypeptide with phase behavior comprises an amino acid sequence selected from:

(a)
(SEQ ID NO: 144)
(GVGVPGVGVPGAGVPGVGVPGVGVP)ₘ;
or
(b)
(SEQ ID NO: 146)
(GVGVPGVGVPGLGVPGVGVPGVGVP)ₘ;

wherein m is an integer between 2 and 32, inclusive of endpoints.

19. The fusion protein of any one of embodiments 1-13, wherein the polypeptide with phase behavior comprises an amino acid sequence selected from:
(a) (GVGVPGVGVPGAGVPGVGVPGVGVP)ₘ (SEQ ID NO: 144), wherein m is 8 or 16;
(b) (GVGVPGAGVP)ₘ (SEQ ID NO: 145), wherein m is an integer between 5 and 80, inclusive of endpoints; or
(c) (GXGVP)ₘ (SEQ ID NO: 147),
wherein m is an integer between 10 and 160, inclusive of endpoints, and
wherein X for each repeat is independently selected from the group consisting of glycine, alanine, valine, isoleucine, leucine, phenylalanine, tyrosine, tryptophan, lysine, arginine, aspartic acid, glutamic acid, and serine.

20. The fusion protein of embodiment 1, wherein the polypeptide with phase behavior comprises an amino acid of SEQ ID NO: 88 or a sequence of (GVGVPGLGVPGVGVPGLGVPGVGVP)ₘ, wherein m is 16 (SEQ ID NO: 12).

21. The fusion protein of any one of embodiments 1 to 20, wherein the fusion protein comprises a linker that links the first polypeptide and the second polypeptide.

22. The fusion protein of embodiment 21, wherein the linker is cleavable.

23. The fusion protein of embodiment 21, wherein the linker comprises a protease cleavage site.

24. The fusion protein of embodiment 21, wherein the linker is a self-cleaving peptide.

25. The fusion protein of embodiment 21, wherein the linker is selected from the group consisting of:

i)
(SEQ ID NO: 141)
(GₓS)ₙ,
ii)
(SEQ ID NO: 142)
(SₓG)ₙ,
iv)
(SEQ ID NO: 19)
(GGGGS)ₙ,
and
v)
(SEQ ID NO: 48)
(G)ₙ;

wherein x is an integer in the range of 1 to 6, and
n is an integer in the range of 1 to 30.

26. The fusion protein of embodiment 21, wherein the linker is selected from GKSSGSGSESKS (SEQ ID NO: 157), GSTSGSGKSSEGKG (SEQ ID NO: 158), GSTSGSGKSSEGSGSTKG (SEQ ID NO: 159), GSTSGSGKPGSGEGSTKG (SEQ ID NO: 160), EGKSSGSGSESKEF (SEQ ID NO: 161), SRSSG (SEQ ID NO: 162), and SGSSC (SEQ ID NO: 163).

27 The fusion protein of any one of embodiments 1-26, wherein the protein comprises from N-terminus to C-terminus, the first polypeptide and the second polypeptide.

28. The fusion protein of any one of embodiments 1-26, wherein the protein comprises from N-terminus to C-terminus, the second polypeptide and the first polypeptide.

29. The fusion protein of any one of embodiments 21-26, wherein the protein comprises from N-terminus to C-terminus, the first polypeptide, the linker, and the second polypeptide.

30. The fusion protein of any one of embodiments 21-26, wherein the protein comprises from N-terminus to C-terminus, the second polypeptide, the linker, and the first polypeptide.

31. A method for stabilizing a first polypeptide, the method comprising expressing a fusion protein comprising the first polypeptide and a second polypeptide, wherein the second polypeptide is a polypeptide having phase behavior.

32. The method of embodiment 31, wherein the fusion protein is the fusion protein of any one of embodiments 1-30.

33. A method for substantially preventing the unfolding, degradation, and/or misfolding of a first polypeptide, the method comprising expressing a fusion protein comprising the first polypeptide and a second polypeptide, wherein the second polypeptide is a polypeptide having phase behavior.

34. The method of embodiment 33, wherein the fusion protein is the fusion protein of any one of embodiments 1-30.

35. A method for substantially preventing loss of activity of a first polypeptide after exposure to one or more conditions known to unfold, degrade, and/or misfold the first polypeptide, the method comprising expressing a fusion protein comprising the first polypeptide and a second polypeptide, wherein the second polypeptide is a polypeptide having phase behavior.

36. The method of embodiment 35, wherein the fusion protein is the fusion protein of any one of embodiments 1-30.

37. The method of embodiment 35 or 36, wherein less than 25%, less than 20%, less than 15%, less than 10%, less than 5%, less than 3%, less than 2%, or less than 1% of the activity of the first polypeptide is lost after exposure to one or more of the conditions.

38. A method for producing a first polypeptide, the method comprising:
i) expressing a fusion protein comprising the first polypeptide and a second polypeptide having phase behavior; and
ii) separating the first polypeptide from the second polypeptide.

39. The method of embodiment 38 wherein the fusion protein is expressed in a host cell selected from a mammalian cell, a bacterial cell, a fungal cell, a yeast cell, and a plant cell.

40. The method of embodiment 39, wherein the yield of the first polypeptide is greater than 15 mg per liter, greater than 30 mg per liter, greater than 50 mg per liter, greater than 100 mg per liter, greater than 200 mg per liter, or greater than 200 mg per liter of host cell suspension.

41. A method for purifying a first polypeptide, the method comprising:
i) providing a fusion protein comprising the first polypeptide and a second polypeptide having phase behavior;
ii) applying a first environmental factor to the fusion protein;
iii) separating the fusion protein aggregates from at least one contaminant on the basis of size and/or density;
iv) applying a second environmental factor to disaggregate the fusion protein.

42. The method of embodiment 41, wherein the fusion protein is the fusion protein of any one of embodiments 1-30.

43. The method of embodiment 41 or 42, wherein the first environmental factor and the second environmental each comprise:
a. a change in one or more of temperature, pH, salt concentration or pressure;
b. the addition of one or more surfactants, cofactors, vitamins, molecular crowding agents, enzymes, denaturing agents; or
c. the application of electromagnetic or acoustic waves.

44. The method of any one of embodiments 41-43, wherein separating the fusion protein aggregates from at least one contaminant comprises separation on the basis of size.

45. The method of embodiment 44, wherein separation on the basis of size is achieved using a technique selected from the group consisting of tangential flow filtration, analytical ultracentrifugation, membrane chromatography, high performance liquid chromatography, normal flow filtration, depth filtration, acoustic wave separation, centrifugation, counterflow centrifugation, and fast protein liquid chromatography

46. The method of any one of embodiments 41-45, wherein the method comprises:
v) separating the first polypeptide from the second polypeptide.

47. The method of any one of embodiments 41-46, wherein the first environmental factor and the second environmental factor are the same.

48. The method of any one of embodiments 41-46, wherein the first environmental factor and the second environmental factor are applied at the same time.

49. A method for performing a multi-step enzymatic process on a substrate, the method comprising:
i) providing a first fusion protein comprising a first enzyme and a polypeptide having a first phase behavior;
ii) providing a second fusion protein comprising a second enzyme and polypeptide having a second phase behavior;
iii) applying a first environmental factor, which allows the first enzyme to contact the substrate; and
iv) applying a second environmental factor, which allows the second enzyme to contact the substrate.

50. The method of embodiment 49, wherein at least one of the first fusion protein and the second fusion protein is a fusion protein of any one of embodiments 1-30.

51. The method of embodiment 49 or 50, wherein the method further comprises at least one of:
v) applying a third environmental factor, which separates the first enzyme from the substrate;
vi) applying a fourth environmental factor, which separates the second enzyme from the substrate.

52. The method of any one of embodiments 49-51, wherein the first environmental factor and the second environmental each comprise:
a. a change in one or more of temperature, pH, salt concentration or pressure;
b. the addition of one or more surfactants, cofactors, vitamins, molecular crowding agents, enzymes, denaturing agents; or
c. the application of electromagnetic waves.

52. A method for performing a multi-step enzymatic process on a substrate, the method comprising:
i) providing a first fusion protein comprising a first enzyme and a polypeptide having a first phase behavior;
ii) providing a second fusion protein comprising a second enzyme and polypeptide having a second phase behavior;
iii) applying a first environmental factor, which allows the first enzyme to contact, isolate, and/or concentrate the substrate; and
iv) applying a second environmental factor, which allows the second enzyme to contact, isolate, and/or concentrate the substrate.

53. The method of embodiment 52, wherein at least one of the first fusion protein and the second fusion protein is a fusion protein of any one of embodiments 1-30.

54. The method of embodiment 52 or 53, wherein the method further comprises at least one of:
v) applying a third environmental factor, which separates the first enzyme from the substrate;
vi) applying a fourth environmental factor, which separates the second enzyme from the substrate.

55. The method of any one of embodiments 52-54, wherein the first environmental factor and the second environmental each comprise:
a. a change in one or more of temperature, pH, salt concentration or pressure;
b. the addition of one or more surfactants, cofactors, vitamins, molecular crowding agents, enzymes, denaturing agents; or
c. the application of electromagnetic waves.

56. A method for contacting, isolating, and/or purifying a substrate, the method comprising:
i) providing a first fusion protein comprising a first enzyme and a polypeptide having a first phase behavior;
ii) providing a second fusion protein comprising a second enzyme and polypeptide having a second phase behavior;
iii) applying a first environmental factor, which allows the first enzyme to contact, isolate, and/or concentrate the substrate; and
iv) applying a second environmental factor, which allows the second enzyme to contact, isolate, and/or concentrate the substrate.

57. The method of embodiment 56, wherein at least one of the first fusion protein and the second fusion protein is a fusion protein of any one of embodiments 1-30.

58. The method of embodiment 56 or 57, wherein the method further comprises at least one of:
v) applying a third environmental factor, which separates the first enzyme from the substrate;
vi) applying a fourth environmental factor, which separates the second enzyme from the substrate.

59. The method of any one of embodiments 56-59, wherein the first environmental factor and the second environmental each comprise:
a. a change in one or more of temperature, pH, salt concentration or pressure;
b. the addition of one or more surfactants, cofactors, vitamins, molecular crowding agents, enzymes, denaturing agents; or
c. the application of electromagnetic waves.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that it constitutes valid prior art or form part of the common general knowledge in any country in the world.

What is claimed is:

1. A method for stabilizing a first polypeptide, the method comprising expressing a fusion protein comprising the first polypeptide and a second polypeptide, wherein the second polypeptide is a polypeptide having phase behavior, wherein when the fusion protein is exposed to one or more conditions that would destabilize the first polypeptide, the first polypeptide substantially retains its activity.

2. A method for substantially preventing loss of activity of a first polypeptide after exposure to one or more conditions that unfold, degrade, and/or misfold the first polypeptide, the method comprising expressing a fusion protein comprising the first polypeptide and a second polypeptide, wherein the second polypeptide is a polypeptide having phase behavior.

3. A method for substantially preventing the unfolding, degradation, and/or misfolding of a first polypeptide, the method comprising expressing a fusion protein comprising the first polypeptide and a second polypeptide, wherein the second polypeptide is a polypeptide having phase behavior, wherein when the fusion protein is exposed to one or more conditions that would cause unfolding, degradation, and/or misfolding.

4. A method for improving a yield of a first polypeptide, the method comprising:
i) expressing a fusion protein comprising the first polypeptide and a second polypeptide having phase behavior; and
ii) separating the first polypeptide from the second polypeptide,
wherein the yield of the first polypeptide is improved when expressed as the fusion protein compared to a yield of the first polypeptide when not expressed as a fusion protein.

5. The method of any one of claims 1-3, comprising removing the fusion protein from the conditions and cleaving the first polypeptide from the second polypeptide, wherein the first polypeptide retains its activity compared to a control first polypeptide that has not been exposed to the conditions.

6. The method of any one of claim 1, 2, or 5, wherein the one or more conditions that unfold, degrade, misfold, or destabilize the first polypeptide comprise: exposure to an oxidizing agent, lyophilization, exposure to non-physiologic pH, exposure to a chaotropic agent, exposure to temperature of at least 50° C., exposure to an organic solvent, exposure to urea, exposure to a detergent, exposure to an autoclave, freeze-thaw cycling, heat shock, or a combination thereof.

7. The method of any one of claim 1, 2, 5, or 6, wherein the one or more conditions that unfold, degrade, misfold, or destabilize the first polypeptide comprise: exposure to non-physiologic pH.

8. The method of claim 7, wherein exposure to non-physiologic pH is exposure to acid.

9. The method of claim 8, wherein the acid is guanidine hydrochloride.

10. The method of claim 7, wherein exposure to non-physiologic pH is exposure to base.

11. The method of claim 10, wherein the base is sodium hydroxide or urea.

12. The method of claim 6, wherein the one or more conditions known to unfold, degrade, misfold, or destabilize the first polypeptide are exposure to guanidine hydrochloride, exposure to urea, lyophilization, freeze-thaw cycling, autoclaving, exposure to sodium hydroxide, or exposure to temperature of at least 90° C.

13. The method of any one of claim 1, 2, 5, or 6, wherein the one or more conditions known to unfold, degrade, misfold, or destabilize the first polypeptide is exposure to 0.1 M NaOH.

14. The method of any one of claim 1, 2, 5, 6, or 13, wherein the one or more conditions known to unfold, degrade, misfold, or destabilize the first polypeptide is exposure to 0.1 M NaOH for 30 minutes.

15. The method of any one of claim 1, 2, 5, or 6, wherein the one or more conditions known to unfold, degrade, or destabilize the first polypeptide is exposure to 6 M guanidine hydrochloride.

16. The method of any one of claim 1, 2, 5, or 6, or 15, wherein the one or more conditions known to unfold, degrade, misfold or destabilize the first polypeptide is exposure to 6 M guanidine hydrochloride for 30 minutes.

17. The method of any one of claim 1, 2, 5, or 6, wherein the one or more conditions known to unfold, degrade, misfold or destabilize the first polypeptide is heating to at least 95° C.

18. The method of any one of claim 1, 2, 5, or 6, wherein the condition is heat shock, and wherein heat shock comprises:
heating the fusion protein comprising the first polypeptide to 95° C. for 30 minutes;
placing a container containing the fusion protein on ice, and then
returning the fusion protein to room temperature.

19. The method of any one of claim 1, 2, or 5-18, wherein the fusion protein comprising the first polypeptide is exposed to the one or more conditions for about 15 minutes, about 30 minutes, about 45 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours.

20. The method of any one of claim 1, 2, or 5-18, wherein exposure to the one or more conditions occurs for at least about 30 minutes.

21. The method of any one of claim 1, 2, 6-10, 13, 15, or 17, wherein exposure to the one or more conditions occurs for about 30 minutes to about 12 hours, about 30 minutes to about 11 hours, about 30 minutes to about 10 hours, about 30 minutes to about 9 hours, about 30 minutes to about 8 hours, about 30 minutes to about 7 hours, about 30 minutes to about 6 hours, about 30 minutes to about 5 hours, about 30 minutes to about 4 hours, about 30 minutes to about 3 hours, about 30 minutes to about 2 hours, or about 30 minutes to about 1 hours.

22. The method of any one of claim 1, 2, or 5-21, wherein the activity of the first polypeptide is its affinity for a binding partner of the first polypeptide.

23. The method of any one of claim 1, 2, or 5-21, wherein the first polypeptide is an enzyme, and wherein the activity is k_{cat}.

24. The method of any one of claim 1, 2, or 5-23, wherein less than 35%, less than 30%, less than 25%, less than 20%, less than 15%, less than 10%, less than 5%, less than 3%, less than 2%, or less than 1% of the activity of the first polypeptide is lost after exposure to one or more of the conditions as compared to a control, wherein the control is not exposed to a condition known to unfold, degrade, or destabilize the first polypeptide.

25. The method of any one of claim 1, 2, or 5-24 wherein the first polypeptide retains from 65% to 100% of its activity after exposure to one or more of the conditions as compared to a control.

26. The method claim 25, wherein the first polypeptide retains at least 65%, at least 70%, at least 75%, at least 80%, at least 85%, at least 90%, at least 95%, or 100% of the its activity after exposure to one or more of the conditions as compared to a control.

27. The method of claim 24, wherein less than 20% or less than 25% of the activity of the first polypeptide is lost after exposure to one or more of the conditions as compared to a control, wherein the control is not exposed to a condition known to unfold, degrade, or destabilize the first polypeptide.

28. The method of claim 25, wherein of the first polypeptide retains at least 80% of its activity after exposure to one or more of the conditions as compared to a control compared control.

29. The method of any one of claim 1, 2, or 5-28, wherein the first polypeptide retains its activity at 4° C. for about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 1 month, about 2 months, about 3 months, about 4 months, about 5 months, about 6 months, about 7 months, about 8 months, about 9 months, about 10 months, about 11 months or about 12 months.

30. The method of any one of claim 1, 2, or 5-28, wherein the first polypeptide retains its activity at −20° C. for about 6 months, about 9 months, about 1 year, about 2 years, about 3 years, about 4 years, about 5 years, about 6 years, about 7 years, about 8 years, about 9 years, or about 10 years.

31. The method of claim 4, wherein the yield of the first polypeptide is greater than 15 mg per liter, greater than 30 mg per liter, greater than 50 mg per liter, greater than 75 mg per liter, greater than 100 mg per liter, greater than 200 mg per liter, or greater than 300 mg per liter of host cell suspension.

32. The method of claim 4, wherein the yield of the first polypeptide in the fusion protein is at least about 50%, at least about 75%, at least about 100%, at least about 125%, at least about 150%, at least about 175%, at least about 200%, at least about 225%, at least about 250%, at least about 275%, at least about 300%, at least about 325%, at least about 350%, at least about 375%, at least about 400%, at least about 425%, at least about 450%, at least about 475%, at least about 500%, at least about 525%, at least about 550%, at least about 575%, at least about 600%, at least about 625%, at least about 650%, at least about 675%, at least about 700%, at least about 725%, at least about 750%, at least about 775%, at least about 800%, at least about 825%, at least about 850%, at least about 875%, at least about 900%, at least about 925%, at least about 950%, at least about 975%, at least about 1000%, at least about 1100%, at least about 1125%, at least about 1150%, at least about 1175%, at least about 1200%, at least about 1225%, at least about 1250%, at least about 1275%, at least about 1300%, at least about 1325%, at least about 1350%, at least about 1375%, at least about 1400%, at least about 1425%, at least about 1450%, at least about 1475%, at least about 1500%, at least about 1525%, at least about 1550%, at least about 1575%, at least about 1600%, at least about 1625%, at least about 1650%, at least about 1675%, at least about 1700%, at least about 1725%, at least about 1750%, at least about 1775%, at least about 1800%, at least about 1825%, at least about 1850%, at least about 1875%, at least about 1900%, at least about 1925%, at least about 1950%, at least about 1975%, or at least about 2000% higher than the yield of a first polypeptide when not expressed as a fusion protein.

33. The method of claim 4, wherein the yield of the first polypeptide is greater than 75 mg per liter.

34. The method of claim 4, wherein the yield of the first polypeptide is about 300% higher than the yield of a first polypeptide that is not expressed as a fusion protein.

35. The method of any one of claims 1-34, wherein the fusion protein is expressed in a host cell selected from a mammalian cell, a bacterial cell, a fungal cell, a yeast cell, and a plant cell.

36. The method of any one of claims 1-35, wherein the first polypeptide is:
i) an enzyme, or catalytic fragment thereof;
ii) an antibody or antigen-binding fragment thereof;
iii) a signaling molecule, or a fragment thereof;
iv) a structural protein, or a fragment thereof;
v) a hormone, or a fragment or derivative thereof;
vi) a nucleic acid binding protein, or a fragment or derivative thereof;
vii) a therapeutic, or a fragment thereof;
viii) a carrier protein, or a fragment thereof;
ix) a cytokine, or a fragment thereof; or
x) a toxin, or a fragment thereof.

37. The method of any one of claims 1-36, wherein the first polypeptide is a mammalian polypeptide.

38. The method of any one of claims 1-36, wherein the first polypeptide is a viral polypeptide.

39. The method of any one of claims 1-36, wherein the first polypeptide is a bacterial polypeptide.

40. The method of any one of claims 1-36, wherein the first polypeptide is a toxin.

41. The method of any one of claims 1-36, wherein the first polypeptide is an antigenic polypeptide.

42. The method of any one of claims 1-41, wherein the first polypeptide is an enzyme capable of performing one or more steps involved in protein synthesis or modification.

43. The method of any one of claims 1-41, wherein the first polypeptide is an enzyme capable of performing one or more steps involved in DNA synthesis or modification.

44. The method of any one of claims 1-41, wherein the first polypeptide is an enzyme capable of performing one or more steps involved in RNA synthesis or modification.

45. The method of any one of claims 1-41, wherein the first polypeptide is selected from the cluster of differentiation 4 (CD4), the Z-domain of Staphylococcus protein A (SpA Z-domain), low-density lipoprotein receptor (LDLR), albumin binding polypeptide (ABD), coxsackievirus and adenovirus receptor (CAR), fibronectin type III (FN3), poly(A) binding protein (PABP), Z-DNA binding protein 1 (ZBP1), or a fragment or derivative thereof.

46. The method of any one of claims 1-41, wherein the first polypeptide is a polypeptide isolated or derived from SARS-COV-2.

47. The method of claim 46, wherein the first polypeptide is the SARS-COV-2 spike protein, or a fragment or derivative thereof.

48. The method of any one of claims 1-47, wherein the first polypeptide with phase behavior is an elastin-like polypeptide (ELP) or a resilin-like polypeptide (RLP).

49. The method of any one of claims 1-47, wherein the polypeptide with phase behavior comprises a pentapeptide repeat having the sequence (Val-Pro-Gly-Xaa-Gly)ₙ (SEQ ID NO: 10), or a randomized, scrambled analog thereof; wherein Xaa can be any amino acid except proline, wherein n is an integer between 1 and 360, inclusive of endpoints.

50. The method of any one of claims 1-48, wherein the polypeptide with phase behavior comprises an amino acid sequence selected from:
a.
(SEQ ID NO: 1)
(GRGDSPY)ₙ
b.
(SEQ ID NO: 2)
(GRGDSPH)ₙ
c.
(SEQ ID NO: 3)
(GRGDSPV)ₙ
d.
(SEQ ID NO: 4)
(GRGDSPYG)ₙ
e.
(SEQ ID NO: 5)
(RPLGYDS)ₙ
f.
(SEQ ID NO: 6)
(RPAGYDS)ₙ
g.
(SEQ ID NO: 7)
(GRGDSYP)ₙ
h.
(SEQ ID NO: 8)
(GRGDSPYQ)ₙ
i.
(SEQ ID NO: 9)
(GRGNSPYG)ₙ
j.
(SEQ ID NO: 11)
(GVGVP)ₙ;
k.
(SEQ ID NO: 12)
(GVGVPGLGVPGVGVPGLGVPGVGVP)ₘ;
l.
(SEQ ID NO: 13)
(GVGVPGVGVPGAGVPGVGVPGVGVP)ₘ;
m.
(SEQ ID NO: 14)
(GVGVPGWGVPGVGVPGWGVPGVGVP)ₘ;
n.
(SEQ ID NO: 15)
(GVGVPGVGVPGVGVPGVGVPGVGVPGVGVPGEGVPGF
GVPGVGVP)ₘ;
o.
(SEQ ID NO: 16)
(GVGVPGVGVPGVGVPGVGVPGVGVPGVGVPGKGVPGF
GVPGVGVP)ₘ;
and
p.
(SEQ ID NO: 17)
(GAGVPGVGVPGAGVPGVGVPGAGVP)ₘ;
or a randomized, scrambled analog thereof;
wherein:
n is an integer in the range of 20-360, inclusive of endpoints; and
m is an integer in the range of 4-25, inclusive of endpoints.

51. The method of any one of claims 1-48, wherein the polypeptide with phase behavior comprises an amino acid sequence selected from:
(SEQ ID NO: 143)
(GVGVP)ₘ;
(SEQ ID NO: 148)
(ZZPXXXXGZ)ₘ;
(SEQ ID NO: 149)
(ZZPXGZ)ₘ;
(SEQ ID NO: 150)
(ZZPXXGZ)ₘ;
or
(SEQ ID NO: 151)
(ZZPXXXGZ)ₘ,
wherein m is an integer between 10 and 160, inclusive of endpoints,
wherein X if present is any amino acid except proline or glycine, and
wherein Z if present is any amino acid.

52. The method of any one of claims 1-48, wherein the polypeptide with phase behavior comprises an amino acid sequence selected from:
(a)
(SEQ ID NO: 144)
(GVGVPGVGVPGAGVPGVGVPGVGVP)ₘ;
or
(b)
(SEQ ID NO: 146)
(GVGVPGVGVPGLGVPGVGVPGVGVP)ₘ;
wherein m is an integer between 2 and 32, inclusive of endpoints.

53. The method of any one of claims 1-48, wherein the polypeptide with phase behavior comprises an amino acid sequence selected from:
(a) (GVGVPGVGVPGAGVPGVGVPGVGVP)ₘ (SEQ ID NO: 144), wherein m is 8 or 16;
(b) (GVGVPGAGVP)ₘ (SEQ ID NO: 145), wherein m is an integer between 5 and 80, inclusive of endpoints; or
(c) (GXGVP)ₘ (SEQ ID NO: 147),
wherein m is an integer between 10 and 160, inclusive of endpoints, and
wherein X for each repeat is independently selected from the group consisting of glycine, alanine, valine, isoleucine, leucine, phenylalanine, tyrosine, tryptophan, lysine, arginine, aspartic acid, glutamic acid, and serine.

54. The method of any one of claims 1-48, wherein the polypeptide with phase behavior comprises an amino acid of SEQ ID NO: 88 or a sequence of (GVGVPGLGVPGVGVPGLGVPGVGVP)ₘ, wherein m is 16 (SEQ ID NO: 12).

55. The method of any one of claims 1-54, wherein the fusion protein comprises a linker that links the first polypeptide and the second polypeptide.

56. The method of claim 55, wherein the linker is cleavable.

57. The method of claim 55, wherein the linker comprises a protease cleavage site.

58. The method of claim 55, wherein the linker is a self-cleaving peptide.

59. The method of claim 55, wherein the linker is selected from the group consisting of:
i)
(SEQ ID NO: 141)
(GₓS)ₙ,
ii)
(SEQ ID NO: 142)
(SₓG)ₙ,
iv)
(SEQ ID NO: 19)
(GGGGS)ₙ,
and
v)
(SEQ ID NO: 48)
(G)ₙ;
wherein x is an integer in the range of 1 to 6, and
n is an integer in the range of 1 to 30.

60. The method of claim 55, wherein the linker is selected from GKSSGSGSESKS (SEQ ID NO: 157), GSTSGSGKSSEGKG (SEQ ID NO: 158), GSTSGSGKSSEGSGSTKG (SEQ ID NO: 159, GSTSGSGKPGSGEGSTKG (SEQ ID NO: 160), EGKSSGSGSESKEF (SEQ ID NO: 161, SRSSG (SEQ ID NO: 162), and SGSSC (SEQ ID NO: 163).

61. The method of any one of claims 1-60, wherein the fusion protein comprises from N-terminus to C-terminus, the first polypeptide and the second polypeptide.

62. The method of any one of claims 1-60, wherein the fusion protein comprises from N-terminus to C-terminus, the second polypeptide and the first polypeptide.

63. The method of any one of claims 55-60, wherein the protein comprises from N-terminus to C-terminus, the first polypeptide, the linker, and the second polypeptide.

64. The method of any one of claims 55-60, wherein the protein comprises from N-terminus to C-terminus, the second polypeptide, the linker, and the first polypeptide.

65. The method of any one of claim 1-44 or 48-64, wherein the first polypeptide comprises a nucleic acid binding protein (NBP).

66. The method of claim 65, wherein the NBP binds to DNA.

67. The method of claim 65, wherein the NBP binds to RNA.

68. The method of claim 66, wherein the DNA is single stranded.

69. The method of claim 66, wherein the DNA is double stranded.

70. The method of claim 65, wherein the RNA is single stranded.

71. The method of claim 65, wherein the RNA is double stranded.

72. The method of claim 64, wherein the RNA is messenger RNA (mRNA), transfer RNA (tRNA), microRNA, ribosomal RNA (rRNA), small nuclear RNA (snRNA), small interfering RNA (siRNA), or heterogenous nuclear RNA (hnRNA).

73. The method of claim 65, 67, 70 or 72, wherein the NBP binds to an mRNA cap or a poly A tail.

74. The method of claim 65, wherein the NBP is a T7 RNA polymerase, Rnase inhibitor, 2′-O-Methyltransferase, Inorganic Pyrophosphatase, Poly(A) Polymerase, DNase I, Calf intestinal phosphatase, Antarctic phosphatase, D1 subunit of the Vaccinia virus mRNA capping enzyme, Guanine-7-methyltransferase (found in D1 subunit of the Vaccinia virus mRNA capping enzyme), Guanylyltransferase (found in D1 subunit of the Vaccinia virus mRNA capping enzyme), RNA triphosphatase (found in D1 subunit of the Vaccinia virus mRNA capping enzyme), and D12 subunit of vaccinia virus mRNA capping enzyme, a stem-loop binding protein, a heterogenous ribonucleoprotein (hnRNP), GroEL, Edc3, DHX9, Xrn1, Dcp1, Dcp2, LAF-1, MEG-1, MEG-3, ASF/SF2 splicing factor, serine/arginine rich splicing factor 4 (SRp75), the serine and arginine rich splicing factor 1 (SRSF1), the L3 ribosomal protein, the L4 ribosomal protein, the L13 ribosomal protein, the L20 ribosomal protein, the L22 ribosomal protein, the L24 ribosomal protein, the L24e ribosomal protein, the S12 ribosomal protein, the S14 ribosomal protein, and the eukaryotic initiation factor 4E-binding protein 1 (4EBP1), Tat, Rev, RSG-1.2 peptide, poly(A)-binding protein (PABP), eukaryotic translation initiation factor 4E (eIF4E), eukaryotic translation initiation factor3 Subunit D (eIF3D), heterogenous nuclear ribonucleoproteins (hnRNPs), RNA-specific adenosine deaminase 1 (ADAR1), RNA-specific adenosine deaminase 2 (ADAR2), CspB from Bacillus subtilis (Bscscp), Y-box protein 1 cold shock domain (YB1-CSD), a Fox-1 protein (FOX1), poly(A)-binding protein (PABP), Staufen protein, TIS11d, zinc finger protein (ZNF), Z-DNA binding protein 1 (ZBP1), retinoic acid-inducible gene-I (RIG-I) like protein, toll like receptor 7 (TLR7), toll like receptor 8 (TLR3), toll like receptor 8 (TLR8), retinoic acid-inducible gene I (RIG-I), melanoma differentiation-associated protein 5 (MDA5), interferon induced protein with tetratricopeptide repeats 1 (IFIT1), protein kinase R (PKR), an oligoadenylate synthase-like (OASL) protein (e.g., OAS1, OAS2, OAS3, or OASL), ribonuclease E (RNASE E), gamma-interferon-inducible protein Ifi-16 (IF116), or cyclic GMP-AMP synthase (cGAS).

75. The method of claim 65, wherein the NBP comprises one or more of the following domains: a short linear motif (SLIM), an RG[G] repeat, an RGG repeat, a RS/RG rich domain, a K/R basic patch, a molecular recognition feature, a low complexity sequence, an RNA recognition motif, a double-stranded RNA binding domain, a K homology domain, a zinc finger domain (e.g., CCHH ZF domain, a CCCC (Ran-BP2) domain, a CCCH ZF domain), an RGG domain, a Pumillo family domain, a pentatricopeptide domain, a cold shock domain, a helicase domain, a La motif, a Piwi-Argonaute-Zwille (PAZ) domain, a P-element induced wimpy testis, a pseudouridine synthase and archaeosine transglycosylate (PUA), a Pumillo-like repeat (PUM), a ribosomal S1-like (S1), Sm and Like-Sm (Sm/Lsm) repeat, thiouridine synthases and RNA methylases and pseudouridine synthases (THUMP), or a domain with YT521-B homology.

76. A method for performing an enzymatic process on a nucleic acid substrate, the method comprising:
(i) providing a first fusion protein comprising a first enzyme and a first polypeptide having a phase behavior; and
(ii) applying a first environmental factor, which allows the first enzyme to contact the substrate.

77. The method of claim 76, comprising:
(iii) providing a second fusion protein comprising a second enzyme and a second polypeptide having phase behavior;
(iv) applying a second environmental factor, which allows the second enzyme to contact the substrate.

78. The method of claim 76, wherein the method further comprises:
(iii) applying a third environmental factor, which separates the first enzyme from the substrate.

79. The method of claim 77, wherein the method further comprises at least one of:
(v) applying a third environmental factor, which separates the first enzyme from the substrate.
(vi) applying a fourth environmental factor, which separates the second enzyme from the substrate.

80. The method of any one of claims 76-79, wherein the first, second, third, and fourth environmental factor are independently selected from:
a. a change in one or more of temperature, pH, salt concentration or pressure;
b. the addition of one or more surfactants, cofactors, vitamins, molecular crowding agents, enzymes, denaturing agents; or
c. the application of electromagnetic waves.

81. The method of any one of claims 76-80, wherein the first enzyme, second enzyme, or both comprises a nucleic acid binding protein (NBP).

82. The method of claim 81, wherein the first enzyme comprises a NBP that binds to DNA.

83. The method of claim 81, wherein the first enzyme comprises a NBP that binds to RNA.

84. The method of any one of claims 81-83, wherein the second enzyme comprises a NBP that binds to DNA.

85. The method of any one of claims 81-83, wherein the second enzyme comprises a NBP that binds to RNA.

86. The method of any one of claims 81 or 82, wherein the first enzyme is capable of performing one or more steps involved in DNA synthesis or modification.

87. The method of any one of claim 81, 82, or 86, wherein the second enzyme is capable of performing one or more steps involved in DNA synthesis or modification.

88. The method of any one of claim 81 or 83, wherein the first enzyme is capable of performing one or more steps involved in RNA synthesis or modification.

89. The method of any one of claim 81, 83, or 88, wherein the second enzyme is capable of performing one or more steps involved in RNA synthesis or modification.

90. The method of any one of claim 81, 82, 84, 86, or 87, wherein the DNA is single stranded.

91. The method of any one of claim 81, 82, 84, 86, or 87, wherein the DNA is double stranded.

92. The method of any one of claim 81, 83, 85, 88, or 89, wherein the RNA is single stranded.

93. The method of any one of claim 81, 83, 85, 88, or 89, wherein the RNA is double stranded.

94. The method of any one of claim 81, 83, 85, 88, 89, 92, or 93, wherein the RNA is messenger RNA (mRNA), transfer RNA (tRNA), microRNA, ribosomal RNA (rRNA), small nuclear RNA (snRNA), small interfering RNA (siRNA), or heterogenous nuclear RNA (hnRNA).

95. The method of claim 81, wherein the NBP binds to an mRNA cap or a poly A tail.

96. The method of any one of claims 81-95, wherein the NBP is a T7 RNA polymerase, Rnase inhibitor, 2′-O-Methyltransferase, Inorganic Pyrophosphatase, Poly(A) Polymerase, DNase I, Calf intestinal phosphatase, Antarctic phosphatase, D1 subunit of the Vaccinia virus mRNA capping enzyme, Guanine-7-methyltransferase (found in D1 subunit of the Vaccinia virus mRNA capping enzyme), Guanylyltransferase (found in D1 subunit of the Vaccinia virus mRNA capping enzyme), RNA triphosphatase (found in D1 subunit of the Vaccinia virus mRNA capping enzyme), and D12 subunit of vaccinia virus mRNA capping enzyme, a stem-loop binding protein, a heterogenous ribonucleoprotein (hnRNP), GroEL, Edc3, DHX9, Xrn1, Dcp1, Dcp2, LAF-1, MEG-1, MEG-3, ASF/SF2 splicing factor, serine/arginine rich splicing factor 4 (SRp75), the serine and arginine rich splicing factor 1 (SRSF1), the L3 ribosomal protein, the L4 ribosomal protein, the L13 ribosomal protein, the L20 ribosomal protein, the L22 ribosomal protein, the L24 ribosomal protein, the L24e ribosomal protein, the S12 ribosomal protein, the S14 ribosomal protein, and the eukaryotic initiation factor 4E-binding protein 1 (4EBP1), Tat, Rev, RSG-1.2 peptide, poly(A)-binding protein (PABP), eukaryotic translation initiation factor 4E (eIF4E), eukaryotic translation initiation factor3 Subunit D (eIF3D), heterogenous nuclear ribonucleoproteins (hnRNPs), RNA-specific adenosine deaminase 1 (ADAR1), RNA-specific adenosine deaminase 2 (ADAR2), CspB from Bacillus subtilis (Bscscp), Y-box protein 1 cold shock domain (YB1-CSD), a Fox-1 protein (FOX1), poly(A)-binding protein (PABP), Staufen protein, TIS11d, zinc finger protein (ZNF), Z-DNA binding protein 1 (ZBP1), retinoic acid-inducible gene-I (RIG-I) like protein, toll like receptor 7 (TLR7), toll like receptor 8 (TLR3), toll like receptor 8 (TLR8), retinoic acid-inducible gene I (RIG-I), melanoma differentiation-associated protein 5 (MDA5), interferon induced protein with tetratricopeptide repeats 1 (IFIT1), protein kinase R (PKR), an oligoadenylate synthase-like (OASL) protein (e.g., OAS1, OAS2, OAS3, or OASL), ribonuclease E (RNASE E), gamma-interferon-inducible protein Ifi-16 (IF116), or cyclic GMP-AMP synthase (cGAS).

97. The method of claim 81, wherein the NBP comprises one or more of the following domains: a short linear motif (SLIM), an RG[G] repeat, an RGG repeat, a RS/RG rich domain, a K/R basic patch, a molecular recognition feature, a low complexity sequence, an RNA recognition motif, a double-stranded RNA binding domain, a K homology domain, a zinc finger domain (e.g., CCHH ZF domain, a CCCC (Ran-BP2) domain, a CCCH ZF domain), an RGG domain, a Pumillo family domain, a pentatricopeptide domain, a cold shock domain, a helicase domain, a La motif, a Piwi-Argonaute-Zwille (PAZ) domain, a P-element induced wimpy testis, a pseudouridine synthase and archaeosine transglycosylate (PUA), a Pumillo-like repeat (PUM), a ribosomal S1-like (S1), Sm and Like-Sm (Sm/Lsm) repeat, thiouridine synthases and RNA methylases and pseudouridine synthases (THUMP), or a domain with YT521-B homology.

98. The method of any one of claims 81-97, wherein the first polypeptide with phase behavior and the second polypeptide with phase behavior comprise the same sequence.

99. The method of any one of claims 81-97, wherein the first polypeptide with phase behavior and the second polypeptide with phase behavior comprise different sequences.

100. The method of any one of claims 81-99, wherein the first polypeptide with phase behavior is an elastin-like polypeptide (ELP) and the second polypeptide with phase behavior is a resilin-like polypeptide (RLP).

101. The method of any one of claims 81-99, wherein the first polypeptide with phase behavior is a resilin-like polypeptide (RLP) and the second polypeptide with phase behavior is a an elastin-like polypeptide (ELP).

102. The method of any one of claims 81-101, wherein the first and/or second polypeptide with phase behavior independently comprises a pentapeptide repeat having the sequence (Val-Pro-Gly-Xaa-Gly), (SEQ ID NO: 10), or a randomized, scrambled analog thereof; wherein Xaa can be any amino acid except proline, wherein n is an integer between 1 and 360, inclusive of endpoints.

103. The method of any one of claims 81-101, wherein the first and/or second polypeptide with phase behavior comprises an amino acid sequence independently selected from:
a.
(SEQ ID NO: 1)
(GRGDSPY)ₙ
b.
(SEQ ID NO: 2)
(GRGDSPH)ₙ
c.
(SEQ ID NO: 3)
(GRGDSPV)ₙ
d.
(SEQ ID NO: 4)
(GRGDSPYG)ₙ
e.
(SEQ ID NO: 5)
(RPLGYDS)ₙ
f.
(SEQ ID NO: 6)
(RPAGYDS)ₙ
g.
(SEQ ID NO: 7)
(GRGDSYP)ₙ
h.
(SEQ ID NO: 8)
(GRGDSPYQ)ₙ
i.
(SEQ ID NO: 9)
(GRGNSPYG)ₙ
j.
(SEQ ID NO: 11)
(GVGVP)ₙ;
k.
(SEQ ID NO: 12)
(GVGVPGLGVPGVGVPGLGVPGVGVP)ₘ;
l.
(SEQ ID NO: 13)
(GVGVPGVGVPGAGVPGVGVPGVGVP)ₘ;
m.
(SEQ ID NO: 14)
(GVGVPGWGVPGVGVPGWGVPGVGVP)ₘ;
n.
(SEQ ID NO: 15)
(GVGVPGVGVPGVGVPGVGVPGVGVPGVGVPGEGVPGFGVPGVGVP)ₘ;
o.
(SEQ ID NO: 16)
(GVGVPGVGVPGVGVPGVGVPGVGVPGVGVPGKGVPGFGVPGVGVP)ₘ;
and
p.
(SEQ ID NO: 17)
(GAGVPGVGVPGAGVPGVGVPGAGVP)ₘ;
or a randomized, scrambled analog thereof;
wherein:
n is an integer in the range of 20-360, inclusive of endpoints; and
m is an integer in the range of 4-25, inclusive of endpoints.

104. The method of any one of claims 81-101, wherein the first and/or second polypeptide with phase behavior comprises an amino acid sequence independently selected from:
(SEQ ID NO: 143)
(GVGVP)ₘ;
(SEQ ID NO: 148)
(ZZPXXXXGZ)ₘ;
(SEQ ID NO: 149)
(ZZPXGZ)ₘ;
(SEQ ID NO: 150)
(ZZPXXGZ)ₘ;
or
(SEQ ID NO: 151)
(ZZPXXXGZ)ₘ,
wherein m is an integer between 10 and 160, inclusive of endpoints,
wherein X if present is any amino acid except proline or glycine, and
wherein Z if present is any amino acid.

105. The method of any one of claims 81-101, wherein the first and/or second polypeptide with phase behavior comprises an amino acid sequence independently selected from:
(a)
(SEQ ID NO: 144)
(GVGVPGVGVPGAGVPGVGVPGVGVP)ₘ;
or
(b)
(SEQ ID NO: 146)
(GVGVPGVGVPGLGVPGVGVPGVGVP)ₘ;
wherein m is an integer between 2 and 32, inclusive of endpoints.

106. The method of any one of claims 81-101, wherein the first and/or second polypeptide with phase behavior comprises an amino acid sequence independently selected from:
(a) (GVGVPGVGVPGAGVPGVGVPGVGVP)ₘ (SEQ ID NO: 144), wherein m is 8 or 16;
(b) (GVGVPGAGVP)ₘ (SEQ ID NO: 145), wherein m is an integer between 5 and 80, inclusive of endpoints; or
(c) (GXGVP)ₘ (SEQ ID NO: 147),
wherein m is an integer between 10 and 160, inclusive of endpoints, and
wherein X for each repeat is independently selected from the group consisting of glycine, alanine, valine, isoleucine, leucine, phenylalanine, tyrosine, tryptophan, lysine, arginine, aspartic acid, glutamic acid, and serine.

107. The method of any one of claims 81-101, wherein the first and/or second polypeptide with phase behavior comprises an amino acid of SEQ ID NO: 88 or a sequence of (GVGVPGLGVPGVGVPGLGVPGVGVP)ₘ, wherein m is 16 (SEQ ID NO: 12).

108. The method of any one of claims 81-107, wherein the first fusion protein comprises a linker that links the first polypeptide and the second polypeptide.

109. The method of any one of claims 82-108, wherein the second fusion protein comprises a linker that links the first polypeptide and the second polypeptide.

110. The method of claim 108 or 109, wherein the linker is cleavable.

111. The method of claim 108 or 109, wherein the linker comprises a protease cleavage site.

112. The method of claim 108 or 109, wherein the linker is a self-cleaving peptide.

113. The method of claim 108 or 109, wherein the linker is independently selected from the group consisting of:
i)
(SEQ ID NO: 141)
(GₓS)ₙ,
ii)
(SEQ ID NO: 142)
(SₓG)ₙ,
iv)
(SEQ ID NO: 19)
(GGGGS)ₙ,
and
v)
(SEQ ID NO: 48)
(G)ₙ;
wherein x is an integer in the range of 1 to 6, and
n is an integer in the range of 1 to 30.

114. The method of claim 108 or 109, wherein the linker is selected from GKSSGSGSESKS (SEQ ID NO: 157), GSTSGSGKSSEGKG (SEQ ID NO: 158), GSTSGSGKSSEGSGSTKG (SEQ ID NO: 159), GSTSGSGKPGSGEGSTKG (SEQ ID NO: 160), EGKSSGSGSESKEF (SEQ ID NO: 161), SRSSG (SEQ ID NO: 162), and SGSSC (SEQ ID NO: 163).

115. The method of any one of claims 81-114, wherein the first fusion protein and/or second fusion protein comprises from N-terminus to C-terminus, the first polypeptide and the second polypeptide.

116. The method of any one of claims 81-114, wherein the first fusion protein and/or second fusion protein comprises from N-terminus to C-terminus, the second polypeptide and the first polypeptide.

117. The method of any one of claims 108-114, wherein the first fusion protein and/or second fusion protein comprises from N-terminus to C-terminus, the first polypeptide, the linker, and the second polypeptide.

118. The method of any one of claims 108-114, wherein the first fusion protein and/or second fusion protein comprises from N-terminus to C-terminus, the second polypeptide, the linker, and the first polypeptide.
